(19) United States
(12) Patent Application Publication
(10) Pub. No.: US 2005/0043381 A1
(43) Pub. Date: Feb. 24, 2005

(54) Aminopyrazole compounds

(75) Inventors: Michael David Johnson, San Diego, CA (US); Min Teng, San Diego, CA (US); Jinjiang Zhu, San Diego, CA (US)

Correspondence Address: AGOURON PHARMACEUTICALS, INC., 10350 NORTH TORREY PINES ROAD, LA JOLLA, CA, 92037, US

(73) Assignee: AGOURON PHARMACEUTICALS, INC.

(21) Appl. No.: 10/897,849

(22) Filed: Jul. 22, 2004

Related U.S. Application Data
(60) Provisional application No. 60/489,976, filed on Jul. 25, 2003.

(51) Int. Cl. A61K 31/425; C07D 275/02

(52) U.S. Cl. 514/372; 548/214

(57) ABSTRACT

Described herein are aminopyrazole compounds of formula I: 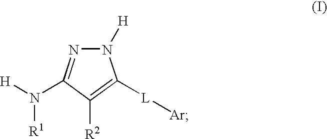
wherein R¹, R², L and Ar are as defined in the specification. Such compounds are capable of modulating the activity of a checkpoint kinase and methods for utilizing such modulation to treat cell proliferative disorders. Also described are pharmaceutical compositions containing such compounds. Also described are the therapeutic or prophylactic use of such compounds and compositions, and methods of treating cancer as well as other diseases associated with unwanted cellular proliferation, by administering effective amounts of such compounds in combination with anti-neoplastic agents.

This application claims the benefit of U.S. Provisional Application No. 60/489,976, filed Jul. 25, 2003.

### FIELD OF INVENTION

Described herein are compositions and methods for modulating the activity of the CHK1 enzyme and for the treatment of disorders in which modulation of the CHK1 enzyme provides benefit to the patient.

### BACKGROUND OF THE INVENTION

The cell cycle is thought to comprise four sequential phases. During this process, cell signals operate to decide the fate of the cell, including proliferation, quiescence, differentiation or apoptosis. See T. Owa, et al., Curr. Med. Chem. 2001, 8, 1487-1503 at 1487.

In order for the cell cycle to function properly, a series of events are initiated, and often completed, in a clearly-defined order. See id. at 1489. Control of the cell cycle is often maintained by certain cell cycle delays or “checkpoints.” Checkpoint enzymes, often kinases, cause a delay in the cell cycle during which important cellular events are completed. Once such events are completed, the cell cycle can be renewed.

One key checkpoint event is the repair of DNA damage prior to DNA replication. If the DNA is not repaired by the cellular machinery, the mutations and damage that have occurred to the DNA prior to replication will be transferred to the daughter cells.

Among the known checkpoint kinases, CHK1 appears to play a significant regulatory role. See T. Owa at 1490; Liu et al, Gene & Dev. 14: 1448-1459 (2000); Takai, et al. Gene & Dev. 14: 1439-1447 (2000); Zachos, G., et al, “CHK1-deficient tumour cells are viable but exhibit multiple checkpoint and survival defects,” EMBO Journal 22: 713-723 (2003). The CHK1 enzyme appears to act by phosphorylating the phosphatase CDC25C. See Sanchez, et al. “Conservation of the CHK1 Checkpoint Pathway in Mammals: Linkage of DNA Damage to Cdk Regulation Through Cdc25,” Science, 1997, 277, 1497-1501; Suganuma, M., et al., “Sensitization of Cancer Cells to DNA Damage-induced Cell Death by Specific Cell Cycle G2 Checkpoint Abrogation,” Cancer Research 59: 5887-5891 (1999); Hutchins, J. R. A., et al. “Substrate specificity determinants of the checkpoint protein kinase CHK1,” FEBS Letters 466: 91-95 (2000); Luo, Y., et al., “Blocking CHK1 Expression Induces Apoptosis and Abrogates the G2 Checkpoint Mechanism,” Neoplasia 3: 411-419 (2001). Another checkpoint kinase, CHK2, has also been identified.

In the treatment of certain diseases, conditions or disorders, damaging the DNA of cells is a desired goal. By modulating the activity of checkpoint kinases, the effect of DNA damaging agents can be enhanced. (See, e.g., Rhind, N. & Russell, P. “CHK1 and Cds1: linchpins of the DNA damage and replication checkpoint pathways,” J. Cell Science 113: 3889-3896 (2000); Sampath, D. & Plunkett, W. “Design of new anticancer therapies targeting cell cycle checkpoint pathways,” Curr. Op. Oncol. 13: 484-490 (2001); Koniaras, K., et al., “Inhibition of CHK1-dependent G2 DNA damage checkpoint radiosensitizes p53 mutant human cells,” Oncogene 20: 7453-7463 (2001); Hapke, G., et al., “Targeting molecular signals in CHK1 pathways as a new approach for overcoming drug resistance,” Cancer and Metastasis Rev. 20: 109-115 (2001); Li, Q. & Zhu, G.-D. “Targeting Seriner/Threonine Protein Kinase B/Akt and Cell-cycle Checkpoint Kinases for Treating Cancer,” Curr. Top. Med. Chem. 2: 939-971 (2002). By way of example only, many treatments for cancer act by damaging DNA of the malignant cells. Because cancer cells are generally highly proliferative compared to normal cells, they are more sensitive to DNA damage. As a result, methods for enhancing DNA damage or limiting the cell's ability to repair the damaged DNA could enhance the effect of DNA-damaging agents.

Compounds which have been asserted to be capable of inhibiting the activity of the CHK1 enzyme have been reported. Many of these inhibitors appear to act by modulating the binding of ATP to CHK1. However, the binding site of ATP to CHK1 is similar to the ATP-binding site of other kinases. Because at least 1000 different kinases are known to be active in the regulation of the cellular machinery (including CHK2, another checkpoint kinase), compounds which inhibit the binding of ATP to the CHK1 enzyme are likely to also inhibit or modulate the activity of other kinases. This lack of selectivity not only limits the amount of inhibitor available to the CHK1 enzyme, but also can lead to numerous unwanted side-effects or adverse reactions.

As a result, inhibitors that have high selectivity for the CHK1 enzyme are needed for the treatment of disorders in which preventing the repair of DNA in a cell would provide benefit to a patient. In this regard, the structure of CHK1, which has been determined by X-ray crystallography, may prove useful. See Chen, P., et al., “The 1.7 Å Crystal Structure of Human Cell Cycle Checkpoint Kinase CHK1: Implications for CHK1 Regulation,” Cell 100: 681-692 (2000).

CHK1 inhibitors have also been described in patents and patent applications. See, e.g., WO 02/070494 “Aryl and Heteroaryl Urea Chk1 Inhibitors For Use as Radiosensitizers and Chamosensitizers” (sic).

All references cited in this section are incorporated by reference in their entirety, and, in particular, as background material to support the statements in the paragraph that contains the citation.

### SUMMARY OF INVENTION

Described herein are compounds capable of modulating the activity of a checkpoint kinase and methods for utilizing such modulation to treat cell proliferative disorders. Also described are aminopyrazole compounds that mediate and/or inhibit the activity of protein kinases, and pharmaceutical compositions containing such compounds. Also described are the therapeutic or prophylactic use of such compounds and compositions, and methods of treating cancer as well as other diseases associated with unwanted angiogenesis and/or cellular proliferation, by administering effective amounts of such compounds.

In one aspect are novel aminopyrazole compounds. In another aspect are compounds in which an aminopyrazole moiety is held in a fixed, linear arrangement with a resorcinol or resorcinol-like moiety. In another aspect are compounds that can modulate the activity of the CHK1 enzyme in vitro and/or in vivo. In yet another aspect are compounds that can selectively modulate the activity of the CHK1 enzyme. In yet another aspect are pharmaceutical compositions of such CHK1-modulating compounds, including pharmaceutically acceptable prodrugs, pharmaceutically active metabolites, or pharmaceutically acceptable salts thereof. In another aspect, the synthesis of such CHK1-modulating compounds, and pharmaceutically acceptable prodrugs, pharmaceutically active metabolites, or pharmaceutically acceptable salts thereof, are described herein. In yet another aspect are methods for modulating the CHK1 enzyme comprising contacting the CHK1-modulating compounds, or pharmaceutically acceptable prodrugs, pharmaceutically active metabolites, or pharmaceutically acceptable salts thereof, described herein, with the CHK1 enzyme. In yet another aspect are methods for treating patients comprising administering a therapeutically effective amount of a CHK1-modulating compound, or a pharmaceutically acceptable prodrug, pharmaceutically active metabolite, or pharmaceutically acceptable salt thereof. In yet another aspect are methods for enhancing the effect of DNA-damaging agents in a patient comprising administering to the patient an enhancing-effective amount of a CHK1-modulating compound, or a pharmaceutically acceptable prodrug, pharmaceutically active metabolite, or pharmaceutically acceptable salt thereof.

In one aspect of the present invention are compounds having the structure of Formula (I): 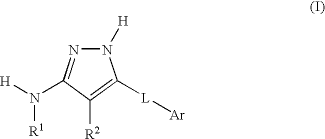
wherein L is a 5- or 6-membered carbocycle or heterocycle group, optionally substituted with 1 to 3 substituents independently selected from the group consisting of Y₁, Y₂ and Y₃;
Ar is a 5- or 6-membered aromatic carbocycle or heterocycle group, optionally substituted with 1 to 3 substituents independently selected from the group consisting of Y₁, Y₂ and Y₃;
R¹ is a moiety selected from the group consisting of —(CR³R⁴)ₜ-aryl, —(CR³R⁴)ₜ-heterocycle, —(CR³R⁴)ₜ—(C₃-C₆)cycloalkyl, (C₂-C₆)alkelnyl, and (C₁-C₆)alkyl, which is optionally substituted with 1 to 3 substituents independently selected from the group consisting of Y₁, Y₂ and Y₃ where t is 0, 1, 2, or 3, wherein when t is 2 or 3, the CR³R⁴ units may be the same or different, and
R² is selected from the group consisting of hydrogen, halogen, and (C₁-C₆)alkyl optionally substituted with 1-3 substituents independently selected from the group consisting of Y₁, Y₂ and Y₃;
R³ and R⁴ are independently selected from the group consisting of H, F, and (C₁-C₆)alkyl, or R³ and R⁴ are selected together to form a carbocycle, or two R³ groups on adjacent carbon atoms are selected together can optionally form a carbocycle;
wherein each Y₁, Y₂, and Y₃ is independently selected and is
   (i) selected from the group consisting of halogen, cyano, nitro, tetrazolyl, guanidino, amidino, methylguanidino, azido, —C(O)Z₁, —CF₃, —CF₂CF₃, —CH(CF₃)₂, —C(OH)(CF₃)₂, —OCF₃, —OCF₂H, —OCF₂CF₃, —OC(O)NH₂, —OC(O)NHZ₁, —OC(O)NZ₁Z₂, —NHC(O)Z₁, —NHC(O)NH₂, —NHC(O)NHZ₁, —NHC(O)NZ₁Z₂, —C(O)OH, —C(O)OZ₁, —C(O)NH₂, —C(O)NHZ₁, —C(O)NZ₁Z₂, —P(O)₃H₂, —P(O)₃(Z₁)₂, —S(O)₃H, —S(O)ₘZ₁, —Z₁, —OZ₁, —OH, —NH₂, —NHZ₁, —NZ₁Z₂, —C(═NH)NH₂, —C(═NOH)NH₂, —N-morpholino, (C₂-C₆)alkenyl, (C₂-C₆)alkynyl, (C₁-C₆)haloalkyl, (C₂-C₆)haloalkenyl, (C₂-C₆)haloalkynyl, (C₁-C₆)haloalkoxy, —(CZ₃Z₄)ᵣNH₂, —(CZ₃Z₄)ᵣNHZ₁, —(CZ₃Z₄)ᵣNZ₁Z₂, and —S(O)ₘ(CF₂)_{q}CF₃, wherein m is 0, 1 or 2, q is an integer from 0 to 5, r is an integer from 1 to 4, Z₁ and Z₂ are independently selected from the group consisting of alkyl of 1 to 12 carbon atoms, cycloalkyl of 3 to 8 carbon atoms, aryl of 6 to 14 carbon atoms, heteroaryl of 5 to 14 ring atoms, aralkyl of 7 to 15 carbon atoms, and heteroaralkyl of 5 to 14 ring atoms; and Z₃ and Z₄ are independently selected from the group consisting of hydrogen, alkyl of 1 to 12 carbon atoms, aryl of 6 to 14 carbon atoms, heteroaryl of about 5 to 14 ring atoms, aralkyl of 7 to 15 carbon atoms, and heteroaralkyl of 5 to 14 ring atoms;
   (ii) Y₁ and Y₂ are selected together to be —O[C(Z₃)(Z₄)]ᵣO— or —O[C(Z₃)(Z₄)]ᵣ₊₁—; or
   (iii) when any two of Y₁, Y₂, or Y₃ are attached to the same or adjacent atoms, they are selected together to form a carbocycle or heterocycle;
and wherein any of the above-mentioned substituents comprising a CH₃ (methyl), CH₂ (methylene), or CH (methine) group which is not attached to a halogen, SO or SO₂ group or to a N, O or S atom optionally bears on said group a substituent selected from hydroxy, halogen, (C₁-C₄)alkyl, (C₁-C₄)alkoxy and —N[(C₁-C₄)alkyl][(C₁-C₄)alkyl];
or a pharmaceutically acceptable prodrug, pharmaceutically active metabolite, pharmaceutically acceptable solvate or pharmaceutically acceptable salt thereof.

In another embodiment are compounds, pharmaceutically acceptable prodrugs, pharmaceutically active metabolites, pharmaceutically acceptable solvates or pharmaceutically acceptable salts having the structure of Formula (I), wherein R¹ is a 5- or 6-membered aryl or heteroaryl group, optionally substituted with 1-3 substituents independently selected from the group consisting of Y₁, Y₂ and Y₃.

In another embodiment are compounds, pharmaceutically acceptable prodrugs, pharmaceutically active metabolites, pharmaceutically acceptable solvates or pharmaceutically acceptable salts having the structure of Formula (I) where Ar is selected to give a structure of Formula (II): 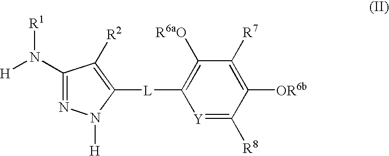
wherein Y is CR⁵ or N;
R^{6a} and R^{6b} are selected from the group consisting of H, —C(O)R⁹, —C(O)OR¹⁰, —C(O)NR⁹R¹⁰ and a moiety selected from the group consisting of —(CR³R⁴)ᵤ-aryl, —(CR³R⁴)ᵤ-heterocycle, —(CR³R⁴)ᵤ—(C₃-C₆)cycloalkyl, (C₂-C₆)alkenyl, and (C₁-C₆)alkyl, optionally substituted with 1 to 3 substituents independently selected from the group consisting of Y₁, Y₂ and Y₃; where u is 0, 1, 2, or 3, wherein when u is 2 or 3, the CR³R⁴ units may be the same or different;
each of R⁵, R⁷, and R⁸ is independently selected from the group consisting of H, halogen, methyl, ethyl, —CN, —CF₃, and —C(O)CH₃;
each of R⁹ and R¹⁰ is independently selected from the group consisting of —(CR³R⁴)ᵤ-aryl, —(CR³R⁴)ᵤ-heterocycle, —(CR³R⁴)ᵤ—(C₃-C₆)cycloalkyl, (C₂-C₆)alkenyl, and (C₁-C₆)alkyl, optionally substituted with 1 to 3 substituents independently selected from the group consisting of Y₁, Y₂ and Y₃; where u is 0, 1, 2, or 3, wherein when u is 2 or 3, the CR³R⁴ units may be the same or different; and
R₁, R₂, R₃, R₄, Y₁, Y₂ and Y₃ are as defined in connection with Formula (I).

In another embodiment are compounds, pharmaceutically acceptable prodrugs, pharmaceutically active metabolites, pharmaceutically acceptable solvates or pharmaceutically acceptable salts having the structure of Formula (I), wherein L is selected from the group consisting of:

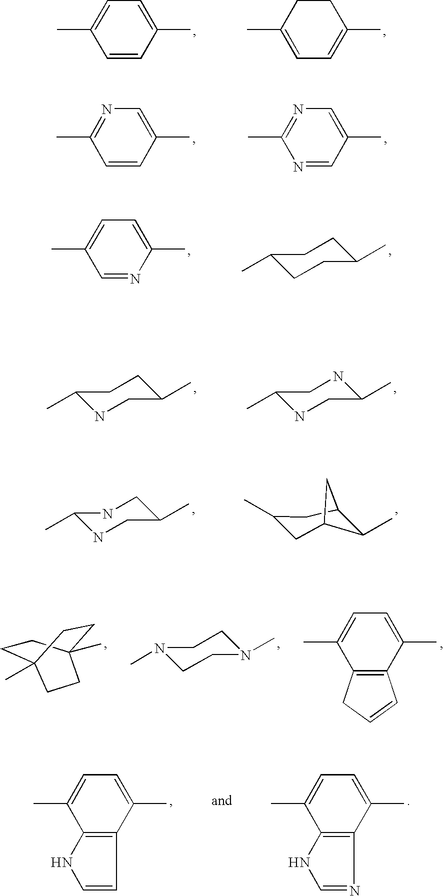

In another embodiment are compounds, pharmaceutically acceptable prodrugs, pharmaceutically active metabolites, pharmaceutically acceptable solvates or pharmaceutically acceptable salts having the structure of Formula (I), wherein L and Ar are each an independently selected optionally substituted phenyl or pyridyl group. Suitable substitutions include substitution with 1 to 3 substituents independently selected from the group consisting of Y₁, Y₂ and Y₃, as defined in connection with Formula (I).

Further are compounds, pharmaceutically acceptable prodrugs, pharmaceutically active metabolites, pharmaceutically acceptable solvates or pharmaceutically acceptable salts having the structure of Formula (I), wherein Ar is: 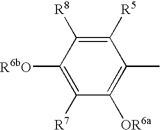
wherein R⁵, R^{6a}, R^{6b}, R⁷ and R⁸ are as defined in connection with Formula (II).

In another embodiment are compounds, pharmaceutically acceptable prodrugs, pharmaceutically active metabolites, pharmaceutically acceptable solvates or pharmaceutically acceptable salts having the structure of Formula (I), wherein L, Ar, and R₂ are selected to give the structure of Formula (III): 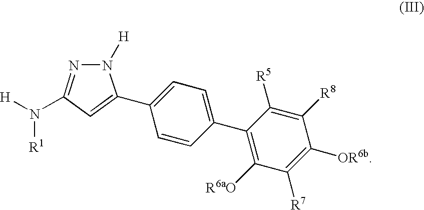
wherein R¹, R⁵, R^{6a}, R^{6b}, R⁷ and R⁸ are as defined in connection with Formulas (I) and (II).

Further are compounds, pharmaceutically acceptable prodrugs, pharmaceutically active metabolites, pharmaceutically acceptable solvates or pharmaceutically acceptable salts having the structure of Formula (III), where R¹ has the structure: 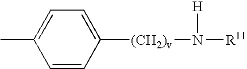
wherein v is 0, 1, or 2; and wherein R¹¹ is (C₁-C₆)alkyl.

In another aspect of the present invention are compounds having the structure of Formula (I), wherein L is Lₐ and Ar is selected to give a structure of Formula (IV): 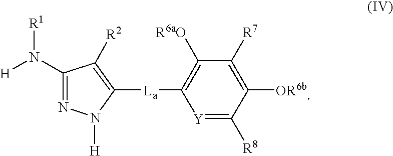
wherein Lₐ is a rigid linking group that orients the aminopyrazole moiety linearly or near-linearly with a resorcinol or resorcinol-like moiety and R¹, R², R^{6a}, R^{6b}, R⁷, R⁸ and Y are as defined in connection with Formulas (I) and (II). According to one embodiment, R^{6a} and R^{6b} are selected from the group consisting of H, —C(O)R⁹, —C(O)OR¹⁰, —C(O)NR⁹R¹⁰ and a moiety selected from the group consisting of (C₃-C₆)cycloalkyl, —(CH₂)ᵤphenyl, —(CH₂)ᵤheterocycle and (C₁-C₄)alkyl which is optionally substituted with 1 to 3 substituents independently selected from the group consisting of Y₁, Y₂ and Y₃, where R⁹ and R¹⁰ are optionally substituted from the group consisting of (C₃-C₆)cycloalkyl, —(CH₂)ᵤphenyl and (C₁-C₆)alkyl which are optionally substituted with 1 to 3 substituents independently selected from the group consisting of Y₁, Y₂ and Y₃; and each of R⁵, R⁷ and R⁸ are independently hydrogen or halogen.

In another aspect are compounds having the structure of Formula (I) selected from the group consisting of:
3-{[3-(2′,4′-Dihydroxy-1,1′-biphenyl-4-yl)-1H-pyrazol-5-yl]amino}benzonitrile;
4-{[3-(2′,4′-Dihydroxy-1,1′-biphenyl-4-yl)-1H-pyrazol-5-yl]amino}benzonitrile;
4′-[5-(3-Cyclopropylaminomethyl-phenylamino)-2H-pyrazol-3-yl]-biphenyl-2,4-diol acetate;
4′-[5-(4-Cyclopropylaminomethyl-phenylamino)-2H-pyrazol-3-yl]-biphenyl-2,4-diol acetate;
4′-[5-(4-isoPropylaminomethyl-phenylamino)-2H-pyrazol-3-yl]-biphenyl-2,4-diol acetate;
4′-[5-(4-N-Cyclopropylaminomethyl-phenylamino)-2H-pyrazol-3-yl]-biphenyl-5-methyl-2,4-diol;
4′-[5-(4-N-Cyclopropylaminomethyl-phenylamino)-2H-pyrazol-3-yl]-biphenyl-6-methyl-2,4-diol;
4′-[5-(4-Cyclopropylaminomethyl-phenylamino)-2H-pyrazol-3-yl]-biphenyl-6-chloro-2,4-diol acetate;
4′-[5-({4-[(cyclopropylamino)methyl]phenyl}amino)-1H-pyrazol-3-yl]-6-fluoro-1,1′-biphenyl-2,4-diol;
4′-[5-(4-Cyclopropylmethylaminomethyl-phenylamino)-2H-pyrazol-3-yl-]-biphenyl-2,4-diol acetate;
N-[3-(2′,4′-dihydoxy-1,1′-biphenyl-4-yl)-1H-pyrazol-5-yl]pyrimidin-2-amine;
4′-[5-(6-Hydroxymethyl-pyridin-3-ylamino)-2H-pyrazol-3-yl]-biphenyl-2,4-diol;
4′-[5-(2-Hydroxymethyl-pyridin-4-ylamino)-2H-pyrazol-3-yl]-biphenyl-2,4-diol acetate;
4′-[5-({6-[(cyclopentylamino)methyl]pyridin-3-yl}amino)-1H-pyrazol-3-yl]-1,1′-biphenyl-2,4-diol;
4′-[5-({6-[(dimethylamino)methyl]pyridin-3-yl}amino)-1H-pyrazol-3-yl]-1,1′-biphenyl-2,4-diol;
5-[5-(2′,4′-Dihydroxy-biphenyl-4-yl)-2H-Pyrazol-3-ylamino]-pyridine-2-carbothioic acid methylamide;
N-[5-(2′,4′-Dihydroxy-1,1′-biphenyl-4-yl)-1H-pyrazol-3-yl]pyridin-2-amine;
N-[5-(2′,4′-Dihydroxy-1,1′-biphenyl-4-yl)-1H-pyrazol-3-yl]pyridin-3-amine;
4′-[5-(pyridin-4-ylamino)-1H-pyrazol-3-yl]-1,1′-biphenyl-2,4-diol;
4′-[5-(1,3-thiazol-5-ylamino)-1H-pyrazol-3-yl]-1,1′-biphenyl-2,4-diol;
4′-(3-anilino-1H-pyrazol-5-yl)-1,1′-biphenyl-2,4-diol;
4′-{5-[(6-{[(cyclopropylmethyl)amino]methyl}pyridin-3-yl)amino]-1H-pyrazol-3-yl}-1,1′-biphenyl-2,4-diol;
4′-[5-({6-[(cyclopropylamino)methyl]pyridin-3-yl}amino)-1H-pyrazol-3-yl]-1,1′-biphenyl-2,4-diol;
4′-[5-({6-[(isopropylamino)methyl]pyridin-3-yl}amino)-1H-pyrazol-3-yl]-1,1′-biphenyl-2,4-diol; and
-[5-({6-[(ethylamino)methyl]pyridin-3-yl}amino)-1H-pyrazol-3-yl]-1,1′-biphenyl-2,4-diol;
   or a pharmaceutically acceptable prodrug, pharmaceutically active metabolite, pharmaceutically acceptable solvate or pharmaceutically acceptable salt thereof.

In another aspect are compounds having the structure of Formula (I) selected from the group consisting of: 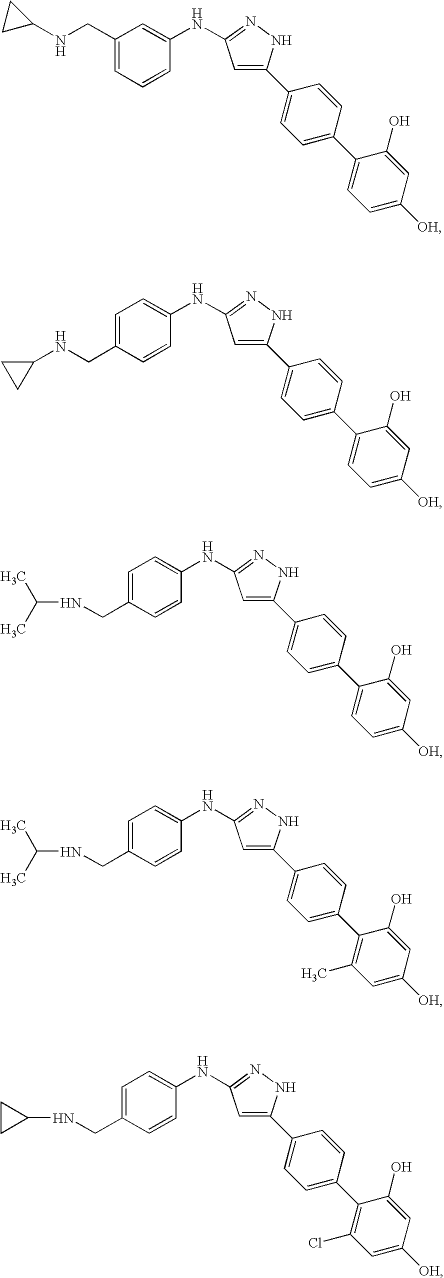 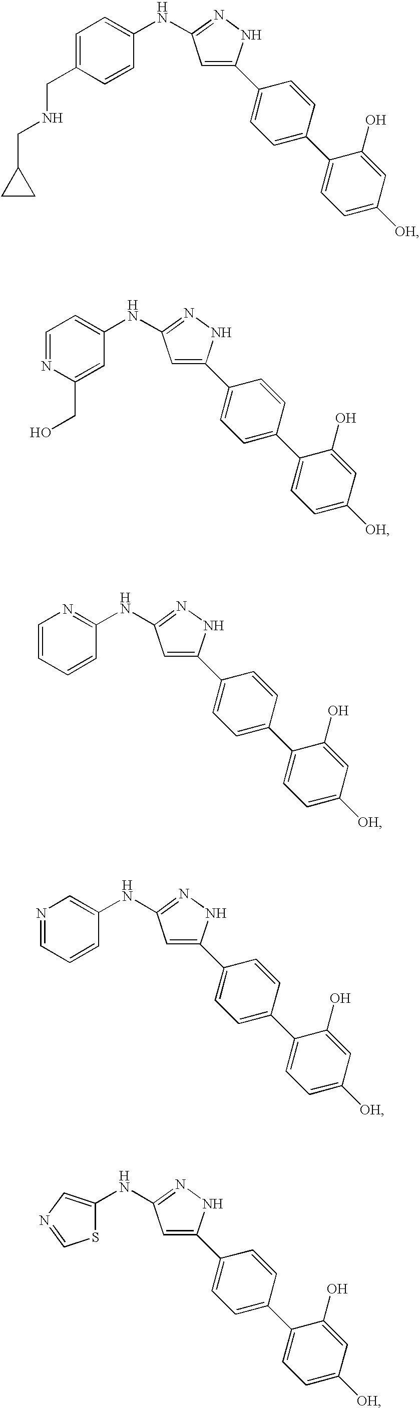 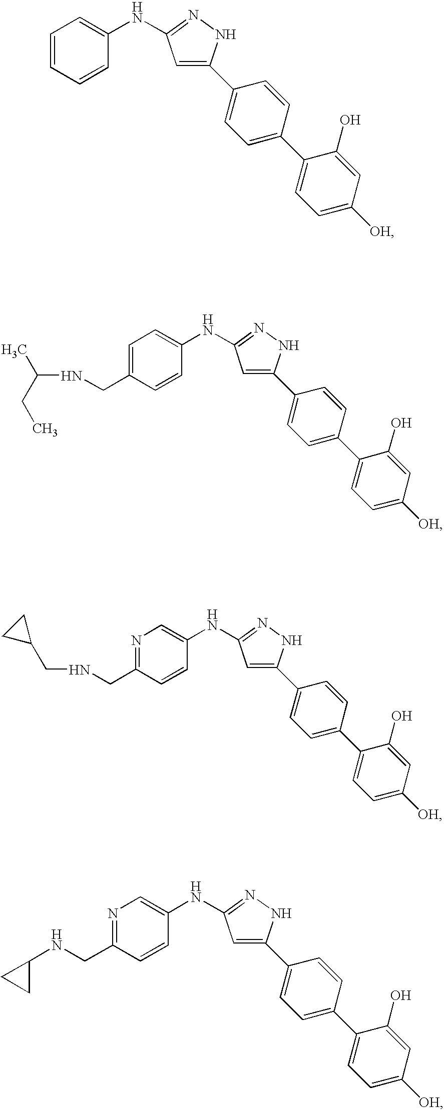 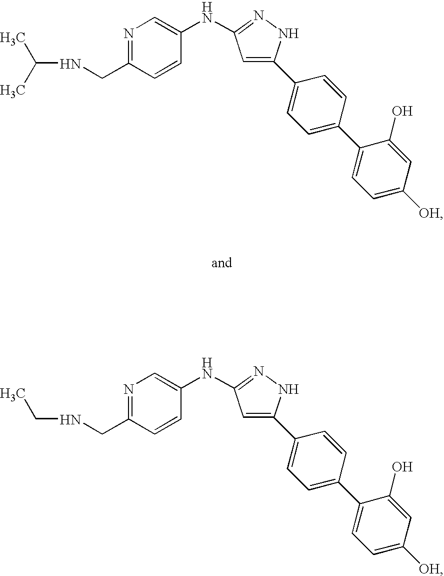
or a pharmaceutically acceptable prodrug, pharmaceutically active metabolite, pharmaceutically acceptable solvate or pharmaceutically acceptable salt thereof.

Another aspect of the present invention is directed to compounds that can modulate the activity of the CHK1 enzyme in vivo or in vitro, wherein the CHK1-modulating compounds have the structure of Formula (I).

Another aspect of the present invention is directed to compounds that can selectively modulate the activity of the CHK1 enzyme over other kinases, wherein the selectivity of the CHK1-modulating compounds for the CHK1 enzyme is at least 10 times higher than for other native kinases.

Another embodiment of the present invention are methods of modulating the activity of a protein kinase receptor, comprising contacting the kinase receptor with an effective amount of a compound having the structure of Formula (I), or a pharmaceutically acceptable prodrug, pharmaceutically active metabolite, pharmaceutically acceptable solvate or pharmaceutically acceptable salt thereof. Further are such methods in which the protein kinase is CHK1.

Another aspect of the invention is to provide a composition for the treatment of neoplasms, and for enhancing the antineoplastic effects of anti-neoplastic agents and therapeutic radiation.

In an embodiment, the invention relates to a composition containing a compound having the structure of Formula (I), a pharmaceutically acceptable salt, solvate, or prodrug thereof and an anti-neoplastic agent as a combined preparation for the simultaneous, separate or sequential use in treating a neoplasm.

In another embodiment, the invention relates to a composition containing a compound having the structure of Formula (I), a pharmaceutically acceptable salt, solvate, or prodrug thereof and an anti-neoplastic agent as a combined preparation for the simultaneous, separate or sequential use in treating a neoplasm wherein the anti-neoplastic agent is selected from the group consisting of alkylating agents, antibiotics and plant alkaloids, hormones and steroids, synthetic agents having anti-neoplastic activity, antimetabolites and biological molecules having anti-neoplastic activity.

In another embodiment, the invention relates to a composition containing a compound having the structure of Formula (I), a pharmaceutically acceptable salt, solvate, or prodrug thereof and an anti-neoplastic agent as a combined preparation for the simultaneous, separate or sequential use in treating a neoplasm wherein the anti-neoplastic agent is selected from the group consisting of Ara-c, VP-16, cis-platin, adriamycin, 2-chloro-2-deoxyadenosine, 9-(3-D-arabinosyl-2-fluoroadenine, carboplatin, gemcitabine, camptothecin, paclitaxel, BCNU, 5-fluorouracil, irinotecan, and doxorubicin.

In another embodiment are pharmaceutical compositions for the treatment of a hyperproliferative disorder in a mammal comprising an enhancing effective amount of a compound having the structure of Formula (I) or a prodrug, metabolite, salt or solvate thereof and a pharmaceutically acceptable carrier. Further are such pharmaceutical compositions, wherein said hyperproliferative disorder is cancer. Further are such pharmaceutical compositions, wherein the cancer is brain, lung, kidney, renal, ovarian, ophthalmic, squamous cell, bladder, gastric, pancreatic, breast, head, neck, oesophageal, gynecological, prostate, colorectal or thyroid cancer. Further are pharmaceutical compositions wherein the hyperproliferative disorder is noncancerous. Further are such pharmaceutical compositions wherein said hyperproliferative disorder is a benign hyperplasia of the skin or prostate.

In another embodiment are pharmaceutical compositions for the treatment of a hyperproliferative disorder in a mammal comprising an enhancing effective amount of a compound having the structure of Formula (I) or a prodrug, metabolite, salt or solvate thereof in combination with an anti-neoplastic agent. Further are such pharmaceutical compositions wherein the anti-neoplastic agent is capable of damaging DNA in a malignant cell. Further are such pharmaceutical compositions wherein the anti-neoplastic agent is selected from the group consisting of mitotic inhibitors, alkylating agents, anti-metabolites, intercalating antibiotics, enzymes, topoisomerase inhibitors, biological response modifiers, anti-hormones, and anti-androgens, and a pharmaceutically acceptable carrier.

In another embodiment are methods of treating a hyperproliferative disorder in a mammal comprising administering to said mammal an enhancing effective amount of a compound having the structure of Formula (I) or a prodrug, metabolite, salt or solvate thereof. Further are such methods wherein said hyperproliferative disorder is cancer. Further are such methods wherein said cancer is brain, lung, ophthalmic, squamous cell, renal, kidney, ovarian, bladder, gastric, pancreatic, breast, head, neck, oesophageal, prostate, colorectal, gynecological or thyroid cancer. Further are such methods wherein said hyperproliferative disorder is noncancerous. Further are such methods wherein said hyperproliferative disorder is a benign hyperplasia of the skin or prostate.

In another embodiment are methods for the treatment of a hyperproliferative disorder in a mammal comprising administering to said mammal an enhancing effective amount of a compound having the structure of Formula (I) or a prodrug, metabolite, salt or solvate thereof in combination with an anti-neoplastic agent. Further are such methods wherein the anti-neoplastic agent is capable of damaging DNA in a malignant cell. Further are such methods wherein the anti-neoplastic agent is selected from the group consisting of mitotic inhibitors, alkylating agents, anti-metabolites, intercalating antibiotics, growth factor inhibitors, cell cycle inhibitors, enzymes, topoisomerase inhibitors, biological response modifiers, anti-hormones, and anti-androgens.

Another aspect of the invention is to provide a method for the treatment of neoplasms.

In another embodiment, the invention relates to a method for treating a neoplasm which comprises administering to a mammal in need thereof, an anti-neoplastic agent in combination with a compound having the structure of Formula (I), a pharmaceutically acceptable salt, solvate, or prodrug thereof, wherein the anti-neoplastic agent is selected from the group consisting of Ara-c, VP-16, cis-platin, adriamycin, 2-chloro-2-deoxyadenosine, 9-p-D-arabinosyl-2-fluoroadenine, carboplatin, gemcitabine, camptothecin, paclitaxel, BCNU, 5-fluorouracil, irinotecan, and doxorubicin. In another embodiment, more than one anti-neoplastic agents may be used in combination with a compound having the structure of Formula (I), the pharmaceutically acceptable salts, solvates, or prodrugs thereof.

Another aspect of the invention is to provide methods for enhancing the anti-neoplastic effect of therapeutic radiation. The CHK-1 inhibitor identified in the present invention may also enhance the anti-neoplasm effects of radiation therapy. Usually, radiation can be used to treat the site of a solid tumor directly or administered by brachytherapy implants. The various types of therapeutic radiation which are contemplated for combination therapy in accordance with the present invention may be those used in the treatment of cancer which include, but are not limited to X-rays, gamma radiation, high energy electrons and High LET (Linear Energy Transfer) radiation such as protons, neutrons, and alpha particles. The ionizing radiation may be employed by techniques well known to those skilled in the art. For example, X-rays and gamma rays are applied by external and/or interstitial means from linear accelerators or radioactive sources. High-energy electrons may be produced by linear accelerators. High LET radiation is also applied from radioactive sources implanted interstitially.

Accordingly, in another embodiment, the invention relates to a method for enhancing the anti-neoplastic effect of therapeutic radiation in a mammal which comprises administering to a mammal in need thereof, a compound having the structure of Formula (I), a pharmaceutically acceptable salt, solvate, or prodrug thereof, in combination with therapeutic radiation having an anti-neoplastic effect.

In an embodiment, the invention relates to a method for treating a neoplasm which comprises administering to a mammal in need thereof, therapeutic radiation having an anti-neoplastic effect in combination with a compound having the structure of Formula (I), a pharmaceutically acceptable salt, solvate, or prodrug thereof.

According to another aspect, the invention provides methods for enhancing the antineoplastic effect of an anti-neoplastic agent.

In an embodiment, the invention relates to a method for enhancing the anti-neoplastic effect of an anti-neoplastic agent in a mammal which comprises administering to a mammal in need thereof, a compound having the structure of Formula (I), a pharmaceutically acceptable salt, solvate, or prodrug thereof, in combination with an antineoplastic agent. The antineoplastic agents include alkylating agents, antibiotics and plant alkaloids, hormones and steroids, synthetic agents having anti-neoplastic activity, antimetabolites and biological molecules having anti-neoplastic activity. Specific antineoplastic agents include Ara-c, VP-16, cis-platin, adriamycin, 2-chloro-2-deoxyadenosine, 9-β-D-arabinosyl-2-fluoroadenine, carboplatin, gemcitabine, camptothecin, paclitaxel, BCNU, 5-fluorouracil, irinotecan, and doxorubicin.

One aspect of the present invention is directed to methods for treating patients comprising administering a therapeutically effective amount of a CHK1-modulating compound, or a pharmaceutically acceptable prodrug, pharmaceutically active metabolite, or pharmaceutically acceptable salt thereof; wherein the CHK1 modulating compound has the structure of Formula (I).

Another aspect of the invention is to provide a method for the treatment of a condition which can be treated by the inhibition of protein kinases. In one embodiment of the invention, the protein kinases are selected from the group consisting of Checkpoint kinase 1 (CHK-1), Checkpoint kinase 2 (CHK-2), Cyclin dependent kinase 1 (CDK1), Serum and glucocorticoid regulated kinase (SGK), Adenosine 5′-monophosphate (AMP)-activated protein kinase (AMPK), Lymphoid T cell tyrosine kinase (LCK), Mitogen activated protein kinase-2 (MAPK-2), Mitogen- and stress-activated protein kinase 1 (MSK1), Rho kinase (ROCK-II), P70 S6 kinase (p70S6K), cAMP (adenosine 3′,5′ cyclic monophosphate)-dependent protein kinase (PKA), Mitogen activated protein kinase (MAPK), Mitogen activated protein kinase-1 (MAPK-1), Protein kinase C-related kinase 2 (PRK2), 3′-Phosphoinositide dependent kinase 1 (PDK1), Fyn kinase (FYN), Protein kinase C (PKC), Protein Kinase C Beta 2 (PKCβII), Protein Kinase C Gamma (PKCγ), Vascular endothelial growth factor receptor 2 (VEGFR-2), Fibroblast growth factor receptor (FGFR), Phosphorylase kinase (PHK), Wee1 kinase (Wee1), and Protein Kinase B (PKB). Preferably, the protein kinases are selected from the group consisting of Checkpoint kinase 1 (CHK-1), Checkpoint kinase 2 (CHK-2), Mitogen activated protein kinase (MAPK), Mitogen activated protein kinase-1 (MAPK-1), Mitogen activated protein kinase-2 (MAPK-2), Vascular endothelial growth factor receptor 2 (VEGFR-2), Fibroblast growth factor receptor (FGFR), Phosphorylase kinase (PHK), Protein Kinase B alpha (PKBα), and Wee1 kinase (Wee1).

In an embodiment, the invention relates to a method for the treatment of a condition which can be treated by the inhibition of protein kinases in a mammal, including a human, comprising administering to a mammal in need thereof, a compound having the structure of Formula (I), a pharmaceutically acceptable salt, solvate, or prodrug thereof.

In another embodiment, said condition which can be treated by the inhibition of protein kinases is selected from the group consisting of connective tissue disorders, inflammatory disorders, immunology/allergy disorders, infectious diseases, respiratory diseases, cardiovascular diseases, eye diseases, metabolic diseases, central nervous system (CNS) disorders, liver/kidney diseases, reproductive health disorders, gastric disorders, skin disorders and cancers.

One aspect of the present invention is directed to methods for enhancing the effect of DNA-damaging agents in a patient comprising administering to the patient an enhancing-effective amount of a CHK1-modulating compound, or a pharmaceutically acceptable prodrug, pharmaceutically active metabolite, or pharmaceutically acceptable salt thereof, wherein the CHK1 modulating compound has the structure of Formula (I).

The subject invention also includes isotopically-labelled compounds, which are identical to those recited in formula (I), but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively. Compounds of the present invention, prodrugs thereof, and pharmaceutically acceptable salts of said compounds or of said prodrugs which contain the aforementioned isotopes and/or other isotopes of other atoms are within the scope of this invention. Certain isotopically-labelled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are noted for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be used in some circumstances. Isotopically labeled compounds of formula (I) of this invention and prodrugs thereof can generally be prepared by carrying out the procedures disclosed in the Schemes and/or in the Examples below, by substituting a readily available isotopically labeled reagent for a non-isotopically labeled reagent.

The compounds of formula (I) or prodrugs thereof, pharmaceutically active metabolites, pharmaceutically acceptable salts, or pharmaceutically acceptable solvates of said compounds and said prodrugs, can each independently also be used in a palliative neo-adjuvant/adjuvant therapy in alleviating the symptoms associated with the diseases recited herein as well as the symptoms associated with abnormal cell growth. Such therapy can be a monotherapy or can be in a combination with chemotherapy and/or immunotherapy.

If the substituents themselves are not compatible with the synthetic methods of this invention, the substituent may be protected with a suitable protecting group that is stable to the reaction conditions used in these methods. The protecting group may be removed at a suitable point in the reaction sequence of the method to provide a desired intermediate or target compound. Suitable protecting groups and the methods for protecting and de-protecting different substituents using such suitable protecting groups are well known to those skilled in the art; examples of which may be found in T. Greene and P. Wuts, Protecting Groups in Chemical Synthesis (3rd ed.), John Wiley & Sons, NY (1999), which is incorporated herein by reference in its entirety. In some instances, a substituent may be specifically selected to be reactive under the reaction conditions used in the methods of this invention. Under these circumstances, the reaction conditions convert the selected substituent into another substituent that is either useful in an intermediate compound in the methods of this invention or is a desired substituent in a target compound.

The compounds of the present invention may have asymmetric carbon atoms. Such diasteromeric mixtures can be separated into their individual diastereomers on the basis of their physical chemical differences by methods known to those skilled in the art, for example, by chromatography or fractional crystallization. Enantiomers can be separated by converting the enantiomeric mixtures into a diastereomeric mixture by reaction with an appropriate optically active compound (e.g., alcohol), separating the diastereomers and converting (e.g., hydrolyzing) the individual diastereomers to the corresponding pure enantiomers. All such isomers, including diastereomeric mixtures and pure enantiomers are considered as part of the invention.

The compounds of present invention may in certain instances exist as tautomers. This invention relates to the use of all such tautomers and mixtures thereof.

Preferably, the compounds of the present invention are used in a form that is at least 90% optically pure, that is, a form that contains at least 90% of a single isomer (80% enantiomeric excess (“e.e.”) or diastereomeric excess (“d.e.”)), more preferably at least 95% (90% e.e. or d.e.), even more preferably at least 97.5% (95% e.e. or d.e.), and most preferably at least 99% (98% e.e. or d.e.).

Additionally, the formulae are intended to cover solvated as well as unsolvated forms of the identified structures. For example, Formula I includes compounds of the indicated structure in both hydrated and non-hydrated forms. Additional examples of solvates include the structures in combination with isopropanol, ethanol, methanol, DMSO, ethyl acetate, acetic acid, or ethanolamine.

In the case of agents that are solids, it is understood by those skilled in the art that the inventive compounds and salts may exist in different crystal or polymorphic forms, all of which are intended to be within the scope of the present invention and specified formulas.

Definitions

As used herein, the following terms have the following meanings, unless expressly indicated otherwise.

The term “acyl” includes alkyl, aryl, or heteroaryl substituents attached to a compound via a carbonyl functionality (e.g., —C(O)-alkyl, —C(O)-aryl, etc.).

The term “acylamino” refers to an acyl radical appended to an amino or alkylamino group, and includes —C(O)—NH₂ and —C(O)—NRR″ groups where R and R′ are as defined in conjunction with alkylamino.

The term “acyloxy” refers to the ester group —OC(O)—R, where R is H, alkyl, alkenyl, alkynyl, or aryl.

The term “alkenyl” refers to optionally substituted unsaturated aliphatic moieties having at least one carbon-carbon double bond and including E and Z isomers of said alkenyl moiety. The term also includes cycloalkyl moieties having at least one carbon-carbon double bond wherein cycloalkyl is as defined above. Examples of alkenyl radicals include ethenyl, propenyl, butenyl, 1,4-butadienyl, cyclopentenyl, cyclohexenyl and the like.

The term “alkenylene” refers to an optionally substituted divalent straight chain, branched chain or cyclic saturated aliphatic group containing at least one carbon-carbon double bond, and including E and Z isomers of said alkenylene moiety.

The term “alkoxy” refers to O-alkyl groups. Examples of alkoxy radicals include methoxy, ethoxy, n-propoxy, isopropoxy, n-butoxy, iso-butoxy, sec-butoxy, tert-butoxy and the like.

The term “alkyl” refers to an optionally substituted saturated monovalent aliphatic radicals having straight, cyclic or branched moieties. Examples of alkyl radicals include methyl, ethyl, n-propyl, isopropyl, n-butyl, isobutyl, sec-butyl, tert-butyl, tert-amyl, pentyl, hexyl, heptyl, octyl and the like.

The term “alkylamino” refers to the —NRR′ group, where R and R′ are independently selected from hydrogen (however, R and R′ cannot both be hydrogen), alkyl, and aryl groups; or R and R′, taken together, can form a cyclic ring system.

The term “alkylene” refers to an optionally substituted divalent straight chain, branched chain or cyclic saturated aliphatic group. The latter group may also be referred to more specifically as a cycloalkylene group.

The term “alkylthio” alone or in combination, refers to an alkyl thio radical, alkyl-S—.

The term “alkynyl” refers to an optionally substituted unsaturated aliphatic moieties having at least one carbon-carbon triple bond and includes straight and branched chain alkynyl groups. Examples of alkynyl radicals include ethynyl, propynyl, butynyl and the like.

The term “amino” refers to the —NH₂ group.

The term “amino acid” refers to both natural, unnatural amino acids in their D and L stereo isomers if their structures allow such stereoisomeric forms, and their analogs. Natural amino acids include alanine (Ala), arginine (Arg), asparagine (Asn), aspartic acid (Asp), cysteine (Cys), glutamine (Gin), glutamic acid (Glu), glycine (Gly), histidine (His), isoleucine (Ile), leucine (Leu), lysine (Lys), methionine (Met), phenylalanine (Phe), proline (Pro), serine (Ser), threonine (Thr), tryptophan (Trp), tyrosine (Tyr) and valine (Val). Unnatural amino acids include, but are not limited to azetidinecarboxylic acid, 2-aminoadipic acid, 3-aminoadipic acid, beta-alanine, aminopropionic acid, 2-aminobutyric acid, 4-aminobutyric acid, 6-aminocaproic acid, 2-aminoheptanoic acid, 2-aminoisobutyric acid, 3-aminoisobutyric acid, 2-aminopimelic acid, 2,4 diaminoisobutyric acid, demosine, 2,2′-diaminopimelic acid, 2,3-diaminopropionic acid, N-ethylglycine, N-ethylasparagine, hydroxylysine, allo-hydroxylysine, 3-hydroxyproline, 4-hydroxyproline, isodesmosine, allo-isoleucine, N-methylglycine, N-methylisoleucine, N-methylvaline, norvaline, norleucine, ornithine and pipecolic acid. Amino acid analogs include the natural and unnatural amino acids which are chemically blocked, reversibly or irreversibly, or modified on their N-terminal amino group or their side-chain groups, as for example, methionine sulfoxide, methionine sulfone, S-(carboxymethyl)-cysteine, S-(carboxymethyl)-cysteine sulfoxide and S-(carboxymethyl)-cysteine sulfone.

The term “aminopyrazole moiety,” as used herein, refers to a group having the structure: 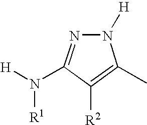
wherein R¹ and R² are substituents such as those defined in connection with Formula (I).

The term “aralkenyl” refers to an alkenyl group substituted with an aryl group. Preferably the alkenyl group has from 2 to about 6 carbon atoms.

The term “aralkyl” refers to an alkyl group substituted with an aryl group. Suitable aralkyl groups include benzyl, phenethyl, and the like. Preferably the alkyl group has from 1 to about 6 carbon atoms.

The term “aryl” refers to an aromatic group which has at least one ring having a conjugated pi electron system and includes a carbocyclic aryl, heterocyclic aryl and biaryl groups, all of which may be optionally substituted.

The term “aryloxy” refers to a group having the formula, R—O—, wherein R is an aryl group.

The term “aralkoxy” refers to a group having the formula, R—O—, wherein R is an aralkyl group.

The term “aromatic” refers to compounds or moieties comprising multiple conjugated double bonds. Examples of aromatic moieties include, without limitation, aryl or heteroaryl ring systems.

The term “arylthio” alone or in combination, refers to an optionally substituted aryl thio radical, aryl-S—.

The term “carbamoyl” or “carbamate” refers to the group —O—C(O)—NRR″ where R and R″ are independently selected from hydrogen, alkyl, and aryl groups; and R and R″ taken together can form a cyclic ring system.

The term “carbocycle” refers to optionally substituted cycloalkyl and aryl moieties. The term “carbocycle” also includes cycloalkenyl moieties having at least one carbon-carbon double bond.

The term “carboxamido” refers to the group 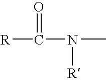
where each of R and R′ are independently selected from the group consisting of H, alkyl, and aryl.

The term “carboxy esters” refers to —C(O)OR where R is alkyl or aryl.

The term “cycloalkyl” refers to optionally substituted saturated monovalent aliphatic radicals having cyclic configurations, including monocyclic, bicyclic, tricyclic, and higher multicyclic alkyl radicals (and, when multicyclic, including fused and bridged bicyclic and spirocyclic moieties) wherein each cyclic moiety has from 3 to about 8 carbon atoms. Examples of cycloalkyl radicals include cyclopropyl, cyclobutyl, cyclopentyl, cyclohexyl and the like.

The terms haloalkyl, haloalkenyl, haloalkynyl and haloalkoxy include alkyl, alkenyl, alkynyl and alkoxy structures, that are substituted with one or more halo groups or with combinations thereof.

The term “halogen” means fluoro, chloro, bromo or iodo. Preferred halogen groups are fluoro, chloro and bromo.

The terms “heteroalkyl” “heteroalkenyl” and “heteroalkynyl” include alkyl, alkenyl and alkynyl radicals and which have one or more skeletal chain atoms selected from an atom other that carbon, e.g., oxygen, nitrogen, sulfur, phosphorus or combinations thereof.

“Heteroaralkyl” refers to an alkyl group substituted with a heteroaryl, such as picolyl, and includes those heterocyclic systems described in “Handbook of Chemistry and Physics”, 49th edition, 1968, R. C. Weast, editor; The Chemical Rubber Co., Cleveland, Ohio. See particularly Section C, Rules for Naming Organic Compounds, B. Fundamental Heterocyclic Systems. Preferably the alkyl group has from 1 to about 6 carbon atoms.

“Heteroaryl” refers to optionally substituted aromatic groups having from 1 to 14 carbon atoms and the remainder of the ring atoms are heteroatoms, and includes those heterocyclic systems described in “Handbook of Chemistry and Physics”, 49th edition, 1968, R.C. Weast, editor; The Chemical Rubber Co., Cleveland, Ohio. See particularly Section C, Rules for Naming Organic Compounds, B. Fundamental Heterocyclic Systems. Suitable heteroatoms include oxygen, nitrogen, and S(O)ᵢ, wherein i is 0, 1 or 2, and suitable heterocyclic aryls include furanyl, thienyl, pyridyl, pyrrolyl, pyrimidyl, pyrazinyl, imidazolyl, and the like.

The term “heterocycle” refers to optionally substituted aromatic and non-aromatic heterocyclic groups containing one to four heteroatoms each selected from O, S and N, wherein each heterocyclic group has from 4 to 10 atoms in its ring system, and with the proviso that the ring of said group does not contain two adjacent O or S atoms. Non-aromatic heterocyclic groups include groups having only 4 atoms in their ring system, but aromatic heterocyclic groups must have at least 5 atoms in their ring system. The heterocyclic groups include benzo-fused ring systems. An example of a 4 membered heterocyclic group is azetidinyl (derived from azetidine). An example of a 5 membered heterocyclic group is thiazolyl. An example of a 6 membered heterocyclic group is pyridyl, and an example of a 10 membered heterocyclic group is quinolinyl. Examples of non-aromatic heterocyclic groups are pyrrolidinyl, tetrahydrofuranyl, dihydrofuranyl, tetrahydrothienyl, tetrahydropyranyl, dihydropyranyl, tetrahydrothiopyranyl, piperidino, morpholino, thiomorpholino, thioxanyl, piperazinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The foregoing groups, as derived from the groups listed above, may be C-attached or N-attached where such is possible. For instance, a group derived from pyrrole may be pyrrol-1-yl (N-attached) or pyrrol-3-yl (C-attached). Further, a group derived from imidazole may be imidazol-1-yl or imidazol-3-yl (both N-attached) or imidazol-2-yl, imidazol-4-yl or imidazol-5-yl (all C-attached). Illustrative examples of (C₂-C₁₀)heterocyclyl are derived from, but not limited to, the following:

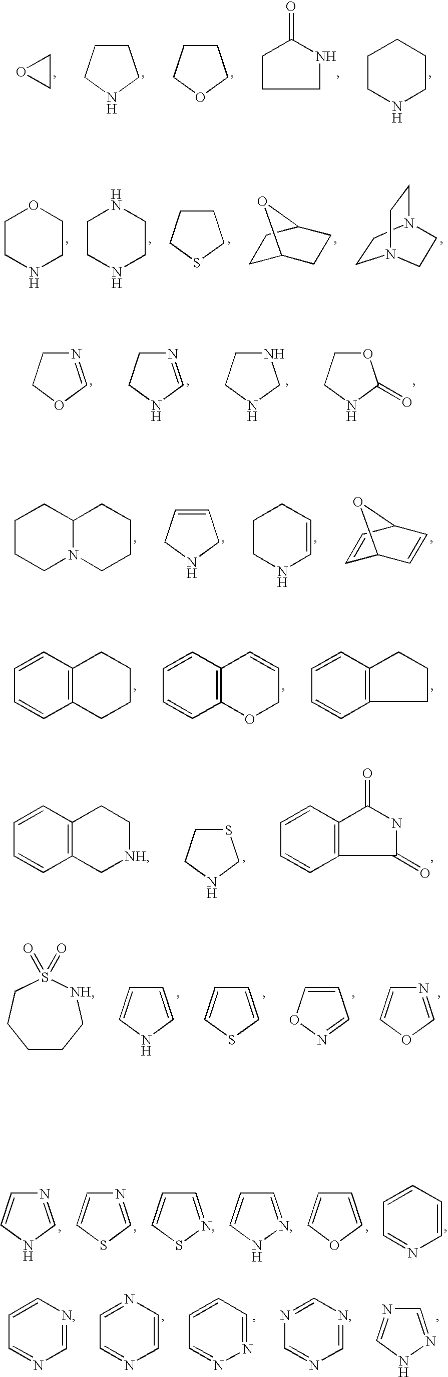

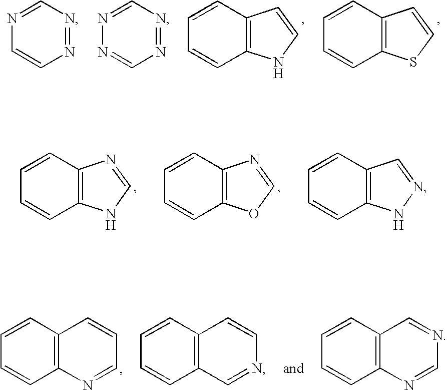

The term “membered ring” can embrace any cyclic structure. The term “membered” is meant to denote the number of skeletal atoms that constitute the ring. Thus, for example, cyclohexyl, pyridine, pyran and thiopyran are 6-membered rings and cyclopentyl, pyrrole, furan, and thiophene are 5-membered rings.

The terms“nucleophile” and “electrophile” as used herein have their usual meanings familiar to synthetic and/or physical organic chemistry. Carbon electrophiles typically comprise one or more alkyl, alkenyl, alkynyl or aromatic (sp³, sp², or sp hybridized) carbon atom substituted with any atom or group having a Pauling electronegativity greater than that of carbon itself. Examples of carbon electrophiles include but are not limited to carbonyls (aldehydes and ketones, esters, amides), oximes, hydrazones, epoxides, aziridines, alkyl-, alkenyl-, and aryl halides, acyls, sulfonates (aryl, alkyl and the like). Other examples of carbon electrophiles include unsaturated carbons electronically conjugated with electron withdrawing groups, examples being the 6-carbon in a β-unsaturated ketones or carbon atoms in fluorine substituted aryl groups. Methods of generating carbon electrophiles, especially in ways which yield precisely controlled products, are known to those skilled in the art of organic synthesis.

In general, carbon electrophiles are susceptible to attack by complementary nucleophiles, including carbon nucleophiles, wherein an attacking nucleophile brings an electron pair to the carbon electrophile in order to form a new bond between the nucleophile and the carbon electrophile.

Suitable carbon nucleophiles include, but are not limited to alkyl, alkenyl, aryl and alkynyl Grignard, organolithium, organozinc, alkyl-, alkenyl, aryl-and alkynyl-tin reagents (organostannanes), alkyl-, alkenyl-, aryl-and alkynyl borane reagents (organoboranes and organoboronates); these carbon nucleophiles have the advantage of being kinetically stable in water or polar organic solvents. Other carbon nucleophiles include phosphorus ylids, enol and enolate reagents; these carbon nucleophiles have the advantage of being relatively easy to generate from precursors well known to those skilled in the art of synthetic organic chemistry. Carbon nucleophiles, when used in conjunction with carbon electrophiles, engender new carbon-carbon bonds between the carbon nucleophile and carbon electrophile.

Nucleophiles suitable for coupling to carbon electrophiles include but are not limited to primary and secondary amines, thiols, thiolates, and thioethers, alcohols, alkoxides, azides, semicarbazides, and the like. These nucleophiles, when used in conjunction with carbon electrophiles, typically generate heteroatom linkages (C—X—C), wherein X is a hetereoatom, e.g, oxygen or nitrogen.

“Optionally substituted” groups may be substituted or unsubstituted. When substituted, the substituents of an “optionally substituted” group may include, without limitation, one or more substituents independently selected from the following groups or designated subsets thereof: (C₁-C₆)alkyl, (C₂-C₆)alkenyl, (C₂-C₆)alkynyl, (C₁-C₆)heteroalkyl, (C₁-C₆)haloalkyl, (C₂-C₆)haloalkenyl, (C₂-C₆)haloalkynyl, (C₃-C₆)cycloalkyl, phenyl, (C₁-C₆)alkoxy, phenoxy, (C₁-C₆)haloalkoxy, amino, (C₁-C₆)alkylamino, (C₁-C₆)alkylthio, phenyl-S—, oxo, (C₁-C₆)carboxyester, (C₁-C₆)carboxamido, (C₁-C₆)acyloxy, H, halogen, CN, NO₂, NH₂, N₃, NHCH₃, N(CH₃)₂, SH, SCH₃, OH, OCH₃, OCF₃, CH₃, CF₃, C(O)CH₃, CO₂CH₃, CO₂H, C(O)NH₂, pyridinyl, thiophene, furanyl, (C₁-C₆)carbamate, and (C₁-C₆)urea. An optionally substituted group may be unsubstituted (e.g., —CH₂CH₃), fully substituted (e.g., —CF₂CF₃), monosubstituted (e.g., —CH₂CH₂F) or substituted at a level anywhere in-between fully substituted and monosubstituted (e.g., —CH₂CF₃).

The term “oxo” means an “O” group.

The term “perhalo” refers to groups wherein every C—H bond has been replaced with a C-halo bond on an aliphatic or aryl group. Examples of perhaloalkyl groups include —CF₃ and —CFCl₂.

The term “resorcinol” or “resorcinol-like moiety,” as used herein, refers to a group having the structure: 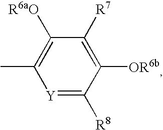
wherein Y, R^{6a}, R^{6b}, R⁷ and R⁸ are substituents such as those defined in connection with Formula II.

The terms “L” or “rigid linking group”, as used herein, refers to a cyclic chemical moiety that allows the aminopyrazole moiety and the resorcinol or resorcinol-like moiety to be in a linear or near-linear orientation. Linear or near-linear refers to an orientation wherein the atoms attached to the rigid-linking group, and the center of the rigid-linking group all lie within the same plane, or nearly (within an angle of +/−10 degrees) the same plane. The rigid linking group may also be optionally substituted. By way of example only, “L” or a “rigid-linking group” can be selected from the following moieties:

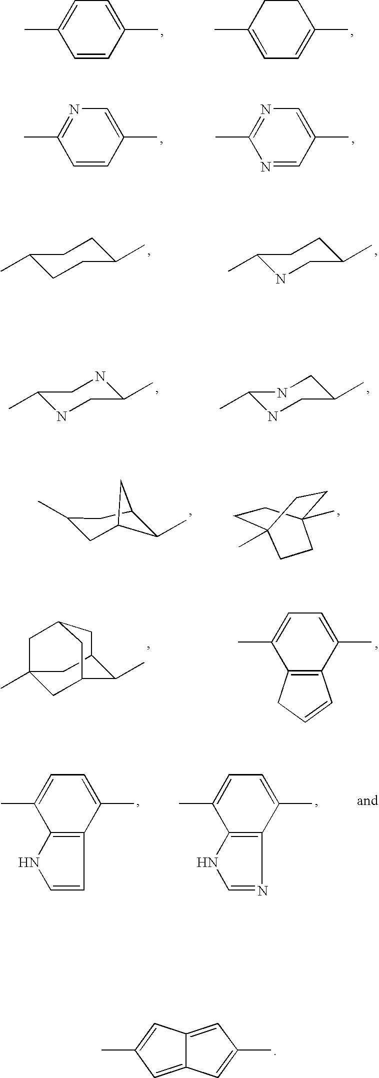

The term “ureyl” or “urea” refers to the group —N(R)—C(O)—NR′R″ where R, R′, and R″ are independently selected from hydrogen, alkyl, aryl; and where each of R—R′, R′—R″, or R—R″ taken together can form a cyclic ring system.

The term “protein kinases” refers to enzymes that catalyze the phosphorylation of hydroxy groups on tyrosine, serine and threonine residues of proteins. The consequences of this seemingly simple activity are staggering; cell growth, differentiation and proliferation, i.e., virtually all aspects of cell life in one way or another depend on the protein kinase activity. Furthermore, abnormal protein kinase activity has been related to a host of disorders, ranging from relatively non-life threatening diseases such as psoriasis to extremely virulent diseases such as glioblastoma (brain cancer). The protein kinases can be conveniently broken down into two major classes, the protein tyrosine kinases (PTKs) and the serine-threonine kinases (STKs). In addition, a third class of dual specificity kinases which can phosphorylate both tyrosine and serine-threonine residues is known. Examples of protein kinases and their isoforms contemplated within this invention include, but are not limited to, Checkpoint kinase 1 (CHK-1), Checkpoint kinase 2 (CHK-2), Cyclin dependent kinase 1 (CDK1), Serum and glucocorticoid regulated kinase (SGK), Adenosine 5′-monophosphate (AMP)-activated protein kinase (AMPK), Lymphoid T cell tyrosine kinase (LCK), Mitogen activated protein kinase-2 (MAPK-2), Mitogen- and stress-activated protein kinase 1 (MSK1), Protein Kinase B (PKB), Protein Kinase B alpha (PKBα), Rho kinase (ROCK-II), P70 S6 kinase (p70S6K), cAMP (adenosine 3′,5′ cyclic monophosphate)-dependent protein kinase (PKA), Mitogen activated protein kinase-1 (MAPK-1), Protein kinase C-related kinase 2 (PRK2), 3′-Phosphoinositide dependent kinase 1 (PDK1), Fyn kinase (FYN), Protein kinase C (PKC), Protein Kinase C Beta 2 (PKCβII), Protein Kinase C Gamma (PKCγ), Vascular endothelial growth factor receptor 2 (VEGFR-2), Fibroblast growth factor receptor (FGFR), Phosphorylase kinase (PHK), Wee1 kinase (Wee1), and Protein Kinase B (PKB).

Checkpoint kinase 2 (CHK-2) acts as a cell cycle checkpoint controller in response to DNA damage. CHK-2 is a downstream effector of ATM which phosphorylates p53 protein and affects cell cycle progression from G₁ to the S phase. CHK-2 activation also affects S phase progression. In addition along with CHK-1, CHK-2 influences G₂/M transition and plays a role in apoptosis if the damage cannot be repaired. CHK-2 could play a role in sensitizing cancer cells to DNA-damaging therapies. CHK-2 may also play a role as a tumor suppressor. Bartek, J. et. al. (2001) Nature Reviews, Molecular Cell biology 2:877-886.

Cyclin dependent kinase 1 (CDK1) is also known as Cdc2 in yeast cells. The cell cycle directs specific events that control growth and proliferation of cells. The cyclin B/Cdk1 complex promotes-entry into mitosis. Cyclin B1 overexpression has been found in 90% of colorectal carcinomas Since the cell cycle is deregulated in human cancers, modulation of CDK activity is a possible therapy. Olomoucine, a CDK inhibitor, has been shown to inhibit cellular proliferation in human cancer cells. In lymphoma cells, olomoucine arrests the cell cycle in both the G₁ and G₂ phases by inhibiting cyclin E/CDK2 and cyclin B/CDK1. Buolamwini, J. K. (2000) current Pharmaceutical Design 6:379-392; Fan, S. et. al. (1999) Chemotherapy 45:437-445.

Serm and glucocorticoid regulated kinase (SGK) is rapidly and highly regulated by corticosteroids in A6 cells at the mRNA and protein levels. SGK is also induced by aldosterone in the kidney of adrenalectomized rats. SGK is activated by 3′-phosphoinositide dependent kinase 1 (PDK1). SGK might play a critical role in aldosterone target cells and may be physiologically important in the early response to aldosterone. Aldosterone receptor antagonists have recently shown great promise in clinical trials for patients with heart failure. The ability to mediate the physiological responses to aldosterone may like-wise prove beneficial. See Leslie, N. R. et. al. (2001) Chemical Reviews 101(8):2365-2380; Funder, J. W. (1999) Molecular and Cellular Endocrinology 151(1-2):1-3; Verrey, F. et. al. (2000) Kidney International 57(4):1277-1282.

Adenosine 5′-monophosphate (AMP)-activated protein kinase (AMPK) isoform α2 (AMPK α2) is present in high concentrations in skeletal muscle, heart, and liver while the α1 isoform is widely distributed. AMPFK, probably the α2 isoform, phosphorylates acetyl-CoA carboxylase β isoform (ACCβ) and inactivates it under conditions electrical stimulation or exercise. In rat skeletal muscle, malonyl-CoA is regulated by ACCβis and involved in the regulatory mechanism of transferring long chain fatty acids into the mitochondria where they are oxidized. AMPK could therefore be linked to obesity and/or insulin resistance, and modulation of AMPK could be potentially beneficial in the treatment of these diseases. AMPK inhibits enzymes involved in glycogen and cholesterol synthesis. It is a possible regulatory enzyme that in response to adenosine 5′-triphosphate (ATP) depletion, reduces further ATP consumption by initiating cellular adjustments that are directed toward maintaining ATP levels. In addition, AMPK has been linked to transcription, regulation of creatinine kinase, apoptosis, and glucose transport. See Kemp, B. E. et. al. (1999) Trends in Biochemical Sciences 24(1):22-25; Friedman, J. (2002) Nature 415(6869):268-269; Ruderman, N. B. et. al. (1999) American Journal of Physiology 276(1, Pt. 1):E1-E18.

Lymphoid T cell tyrosine kinase (LCK) is a cytosolic non-receptor tyrosine kinase and a T-lymphocyte member of the Src family. LCK has been implicated in early phase T-cell receptor activation by antigens and plays a critical role in T-cell mediated immune responses. Upon activation by autophosphorylation, LCK phosphorylates T-cell receptor ξ-chains which can then recruit a second cytoplasmic protein-tyrosine kinase ZAP-70 to promote T-cell activation. Inhibitors could be used for the treatment of rheumatoid arthritis, diseases related to immune response and T-cell based leukemias and lymphomas. See Garcia-Echeverria, C. (2001) Current Medicinal Chemistry 8(13):1589-1604; Majolini, M. B. et. al. (1999) Leukemia & Lymphoma 35(3/4):245-254.

Mitogen- and stress-activated protein kinase 1 (MSK1) is activated on stimulation of the Ras-mitogen activated protein kinase (MAPK) pathway and also by the p38 stress kinase pathway. Both pathways are implicated in tumorigenesis. Stimulation of the Ras-MAPK signal transduction pathway by growth factors or phorbol esters results in phosphorylation of histone H3. MSK1 has been shown to mediate epidermal growth factor (EGF) or TPA (12-O-tetradecanoylphorbol-13-acetate, a phorbol ester) induced phosphorylation of H3. There is evidence that persistent activation of Ras-MAPK pathway and MSK1 resulting in elevated phosphorylated H3 levels may contribute to aberrant gene expression observed in oncogene-transformed cells. Inhibition of MSK1 suppressed the induction of c-fos (proto-oncogene) and uPA genes in parental and oncogene-transformed cells. Both c-fos and uPA are involved in tumor invasion and metastasis. See Strelkov, I. et. al. (2002) Cancer Research 62(1):75-78; Zhong, S. et. al. (2001) Journal of Biological Chemistry 276(35):33213-33219; Nomura, M. et. al. (2001) Journal of Biological Chemistry 276(27);25558-25567.

Rho kinase (ROCK-II) is also known as ROKα. By inhibiting ROCK-II, one could potentially influence Rho GTPases which act as molecular controls that regulate many essential cellular processes, including actin dynamics, cell-cycle progression, and cell adhesion. The in vitro and in vivo biological effects of Y-27632, a specific inhibitor of ROCK, have been described in the literature and include lowering blood pressure in hypertensive rats, inhibition of Rho-induced formation of stress fibers and focal adhesions, and inhibition of tumor growth. See Narumiya, S. et. al (2000) Methods in Enzymology 325 (Regulators and Effectors of Small GTPases, Part D): 273-284 (and associated references listed therein); Bishop, et al. (2000) Biochem. J. 348: 241-255.

P70 S6 kinase (p70^{S6K}) is found as two isoforms-one cytoplasmic and the other in the nucleus. They are similar except for N-terminus, and both are called p70^{S6K} or S6K1. A second functional homologue S6K2 was also identified. p70^{S6K} is a downstream target of the lipid kinase phosphoinositide 3-OH kinase (PI(3)K). P70^{S6K} is implicated in cell cycle control and neuronal cell differentiation. P70^{S6K} may also function in regulating cell motility which could influence tumor metastases, the immune response, and tissue repair. Along with PKB/Akt, p70^{S6K} is a crucial effector in oncogenic protein-tyrosine kinase (PTK) signaling. P70^{S6K} may be a more potent kinase for BAD than PKB/Akt (see above) in response to insulin growth factor 1 (IGF-1) stimulation. P70^{S6K} may therefore play an important antiapoptotic role. See Blume-Jensen, P. et. al. (2001) Nature 411(6835):355-365; Accili, D. (2001) Journal of Clinical Investigation 108(11):1575-1576; Hidalgo, M. et al. (2000) Oncogene 19(56):6680-6686; Berven, L. et. al. (2000) Immunology and Cell Biology 78(4):447-451.

cAMP (adenosine 3′,5′ cyclic monophosphate)-dependent protein kinase (PKA) is involved in a wide range of physiological responses following interaction with cAMP. cAMP is a second messenger that regulates many different cellular activities such as gene transcription, cell growth and differentiation, ion channel conductivity, and release of neurotransmitters. The cAMP/PKA interaction acts as a major regulatory mechanism in mammals, and PKA has been shown phosphorylate a myriad of physiological substrates. PKA has two major isoforms—PKAI and PKAII. PKAI inhibitors have shown enhancing effects when used in combination certain cytotoxic cancer therapies. Antisense oligonucleotides targeting the PKAI subunit RIα have shown enhanced anti-tumor effects when combined with Taxol. Glucagon activates PKA and PKA may influence insulin response along with calmodulin-dependent protein kinase and protein kinase C. PKA is involved in regulating cardiac L-type calcium channels, and modulation of the implicated regulatory pathways may prove useful in the treatment of heart disease. In addition, dysfunctional T-cells isolated from HIV patients have been restored by the addition of PKAI antagonists. See Skalhegg, B. S. et. al. (2000) Frontiers in Bioscience [Electronic Publication] 5:D678-D693; Brandon, E. P. et. al. (1997) Current Opinion in Neurobiology 7(3):397-403; Nesher, R. et. al.(2002) Diabetes 51(Suppl. 1): S68-S73; Shabb, J. B. (2001) Chemical Reviews 101(8):2381-2411; Kamp, T. J. et. al. (2000) Circulation Research 87(12);1095-1102; Tortora, G. et.al. (2002) Clinical Cancer Research 8:303-304; Tortora, G. et. al. (2000) Clinical Cancer Research 6:2506-2512.

Mitogen activated protein kinase (MAPK) is also known as ERK. In tumorigenesis, ras oncogenes transmit extracellular growth signals. The MAPK pathway is an important signaling route between membrane-bound ras and the nucleus. A phosphorylation cascade involving three key kinases is involved. They are Raf, MEK (MAP kinase) and MAPK/ERK. Raf isoforms phosphorylate and activate isoforms MEK1 and MEK2. MEK1 and 2 are dual specificity kinases that in turn phosphorylate and activate the MAPK isoforms MAPK1/ERK1 and MAPK2/ERK2. In fibroblasts, MAPK1/ERK1 and MAPK2/ERK2 are both strongly activated by growth factors and by tumor-promoting phorbol esters. MAPK1/ERK1 and MAPK2/ERK2 are also involved with glucose regulation, neurotransmitter regulation, and secetagogue regulation (in endocrine tissues). The MAPK pathway has also been linked to the induction of cyclin D1 mRNA and thus linked to G1 phase of cell cycle. See Webb, C. P. et. al. (2000) Cancer Research 60(2), 342-349; Roovers, K. et. al. (2000) BioEssays 22(9):818 826; Chen, Z. et. al. (2001) Chemical Reviews 101(8):2449-2476; Lee, J. C. et. al. (2000) Immunopharmacology 47(2-3):185-201, Sebolt-Leopold J. S. (2000) Oncogene 19:6594-6599; Cheng, F. Y. et. al. (2001) Journal of Biological Chemistry 276(35):32552-32558; Cobb, M. H. et. al. (2000) Trends in Biochemical Sciences 25(1):7-9; Cobb, M. H. et. al. (1995) Journal of Biological Chemistry 270(25):14843-14846; Deak, M. et. al. (1998) EMBO Journal 17(15):4426-4441; Davis, J. D. (1993) Journal of Biological Chemistry 268(20):14553-14556.

cSrc (also known as p60 c-src) is cytosolic, non-receptor tyrosine kinase. c-Src is involved in the transduction of mitogenic signals from a number of polypeptide growth factors such as epidermal growth factor (EGF) and platelet-derived growth factor (PDGF). c-Src is over expressed in mammary cancers, pancreatic cancers, neuroblastomas, and others. Mutant c-Src has been identified in human colon cancer. c-Src phosphorylates a number of proteins that are involved in regulating cross-talk between the extracellular matrix and the cytoplasmic actin cytoskeleton. Modulation cSrc activity could have implications in diseases relating to cell proliferation, differentiation and death. See Bjorge, J. D. et. al. (2000) Oncogene 19(49):5620-5635; Halpern, M. S. et. al. (1996) Proc. Natl. Acad. Sci. U. S. A. 93(2), 824-7; Belsches, A. P. et. al. (1997) Frontiers in Bioscience [Electronic Publication] 2:D501-D518; Zhan, X. et. al (2001) Chemical Reviews 101:2477-2496; Haskell, M. D. et. al. (2001) Chemical Reviews 101:2425-2440;

Protein kinase C-related kinase 2 (PRK2) is regulated by the G-protein Rho. PRK2 is found in regions of large actin turnover. Endogenous PRK2 kinase activity increases with keratinocyte differentiation and is associated with keratinocyte cell-cell adhesion and Fyn kinase activation. See Gross, C., et. al. (2001) FEBS Letters 496(2,3):101-104; Calautti, E. et. al. (2002) Journal of Cell Biologyl 56(1):137-148.

3′-Phosphoinositide dependent kinase 1 (PDK1) phosphorylates and activates members of the AGC (cAMP-dependent, cGMP-dependent, and protein kinase C) kinase family that are activated downstream of phosphoinositide 3-kinase (PI3K). PI3K becomes active through insulin stimulation. PDK1 activates a number of protein kinases and therefore can be connected to the regulation of a number of insulin specific events. PDK1 phosphorylation and activation of PKCξ is necessary for insulin-dependent GLUT4 translocation. Insulin-induced GLUT4 translocation is physiologically related to the actin-based cytoskeleton. Disturbances in actin filaments have been linked to loss of insulin effect on glucose transport and decreased translocation of GLUT4. See Wick, K. L. et. al. (2001) Current Drug Targets: Immune, Endocrine and Metabolic Disorders 1(3):209-221; Peterson, R. T. et. al. (1999) Current Biology 9(14):R521-R524; Toker, A. et al. (2000) Cell 103(2):185-188; Leslie, N. R. (2001) Chem. Rev. 101: 2365-2380.

Fyn kinase (FYN) is a member of the Src family of tyrosine kinases. Fyn has been implicated in positive control of keratinocyte cell-cell adhesion. Adhesion plays a crucial function in establishment and maintenance of organized tissues. Fyn knockout and transgenic mice established that Fyn participates in T cell receptor (TCR) signaling. Overexpression of the fyn(T) transgene produces T cells with enhanced responsiveness to TCR signaling. Conversely, expression of an inactive kinase form is inhibitory. Fyn may be an appropriate target for treatment of autoimmune diseases. Fyn −/− mice are hypersensitive to alcohol which suggests that Fyn might be a target for the treatment of alcoholism. Alteration of Fyn levels may also aid in the treatment of skin disorders. Fyn has been implicated in the regulation of programmed cell death, and Fyn−/− mice exhibit reduced apoptosis. See also PRK2. See Calautti, E. et. al. (2002) Journal of Cell Biology 156(1):137-148; Resh, M. D. (1998) Journal of Biochemistry & Cell Biology 30(11):1159-1162.

Vascular endothelial growth factor receptor 2 (VEGFR-2) is also known as FLK-1 and as KDR (kinase insert domain receptor). Other VEGF receptor tyrosine kinases include VEGFR-1(Flt-1) and VEGFR-3 (Flt-4). Angiogenesis or the development of new vasculature is central to the process by which solid tumors grow. The degree of vasculaturization has been linked with increased potential for metastasis. VEGFR-2, expressed only on endothelial cells, binds the potent angiogenic growth factor VEGF and mediates the subsequent signal transduction. Inhibition of VEGF-R2 activity has resulted in decreased angiogenesis and tumor growth in in vivo models, and inhibitors of VEGFR-1 are currently in clinical trials for the treatment of cancer. See Strawn et al.,(1996) Cancer Research 56:3540-3545; Millauer et al.,(1996) Cancer Research 56:1615-1620; Sakamoto, K. M. (2001) IDrugs 4(9):1061-1067; Ellis, L. M. et. al. (2000) Oncologist 5(Suppl. 1):11-15; Mendel, D. B. et. al (2000) Anti-Cancer Drug Design 15:29-41; Kumar, C. C. et. al. (2001)Expert Opin. Emerging Drugs 6(2):303-315; Vajkoczy, P. et. al (1999) Neoplasia 1(1):31-41.

Fibroblast growth factor receptor (FGFR) binds the angiogenic growth factors aFGF and bFGF and mediates subsequent intracellular signal transduction. Growth factors such as bFGF may play a critical role in inducing angiogenesis in solid tumors that have reached a certain size. FGFR is expressed in a number of different cell types throughout the body and may or may not play important roles in normal physiological processes in adult humans. Systemic administration of a small-molecule inhibitor of FGFR has been reported to block bFGF-induced angiogenesis in mice. See Yoshiji et al., (1997) Cancer Research 57: 3924-3928; Mohammad et al., (1998) EMBO Journal 17:5996-5904.

Phosphorylase kinase (PHK) activates glycogen phosphorylase. The primary consequence of this activation is to release glucose 1-phosphate from glycogen. Conversion to glycogen is the major means by which glucose is stored in mammals. Intracellular glycogen stores are used to maintain blood-glucose homeostasis during fasting and are a source of energy for muscle contraction. In Vivo, PHK is phosphorylated by cAMP-dependent protein kinase (PKA) which increases the specific activity of PHK. Both Type 1 and 2 diabetics show reduced glycogen levels in liver and muscle cells. Glycogen levels are tightly regulated by hormones and metabolic signaling. Kinase inhibitors that could augment intracellular glycogen levels may prove beneficial in the treatment of diabetes. See Brushia, R. J. et. al. (1999) Frontiers in Bioscience [Electronic Publication] 4:D618-D641; Newgard, C. B. et. al. (2000) Diabetes 49:1967-1977; Venien-Bryan, C. et. al. (2002) Structure 10:33-41; Graves, D. et. al. (1999) Pharmacol. Ther. 82:(2-3)143-155; Kilimann, M. W. (1997) Protein Dysfunction and Human Genetic Disease Chapter 4:57-75.

Wee1 kinase (Wee1) along with Mik1 kinase has been shown to phosphorylate Cdc2. Phosphorylation of Cdc2 has been shown to prevent mitotic entry. Wee1 may play an important role the normal growth cycle of cells and may be implicated in cell-cycle checkpoint control. Rhind, N. et. al. (2001) Molecular and Cellular Biology 21(5):1499-1508.

Protein Kinase B (PKB) is also known as Akt. There are three very similar isoforms known as PKB α, β, and γ (or Akt 1, 2, and 3). Ultraviolet irradiation in the 290-320 nM range has been associated with the harmful effects of sunlight. This irradiation causes activation of PKB/Akt and may be implicated in tumorigenesis. Over expressed PKB/Akt has been shown in ovarian, prostate, breast & pancreatic cancers. PKB/Akt is also involved in cell cycle progression. PKB/Akt promotes cell survival in a number of ways. It phosphorylates the proapoptotic protein, BAD, so that it is unable to bind and inactivate the antiapoptotic protein Bcl-xl. PKB/Akt also serves to inhibit apoptosis by inhibiting caspase 9 and forkhead transcription factor and by activating IkB kinase. See Barber, A. J. (2001) Journal of Biological Chemistry 276(35):32814-32821; Medema, R. H. et al. (2000) Nature 404:782-787; Muise-Helmericks, R. C. et. al (1998) Journal of Biological Chemistry 273(45): 29864-29872; Nomura, M. et. al. (2001) Journal of Biological Chemistry 276(27): 2558-25567; Nicholson, K. M. et. al. (2002) Cellular Signaling 14(5): 381-395; Brazil, D. P. et. al. (2001) Trends in Biochemical Sciences 26(11): 657-664. Leslie, N. R. (2001) Chem Rev 101: 2365-2380.

Protein kinase C (PKC) classical isoforms are designated α,β1, β2, and γ, and all are Ca²⁺ dependent. PKC isoforms are involved in signal transduction pathways linked to a number of physiological responses including membrane transport, cellular differentiation and proliferation, organization of cytoskeletal proteins and gene expression. Tumor promoting phorbol esters activate classical PKC isoforms and antisense oligonucleotides can block this activation. PKC isoforms are often over expressed in various cancers. PKC inhibitors have been shown to reverse p-glycoprotein-mediated multi-drug resistance and can increase intracellular concentrations of other anti-cancer agents. In myocytes, PKC isoforms have been implicated in certain cardiac pathologies. PKC-γ is highly expressed in brain and spinal cord and is primarily localized in dendrites and neuron cell bodies. PKC-β2 is involved in cell proliferation and overexpression increases sensitivity to cancer. PK\tilde{C}β inhibitors are a potential new therapy for diabetic retinopathy with clinical trials ongoing. See Magnelli, L. et. al. (1997) Journal of Cancer Research and Clinical Oncology 123(7):365-369; Clerk, A. et. al (2001) Circulation Research 89(10): 847-849; Carter, C. (2000) Current Drug Targets1(2):163-183; Greenberg, S. et. al. (1998) Alcohol16(2);167-175; Rosenzweig, T. et. al. (2002) Diabetes51(6):1921-1930; Deucher, A. et. al. (2002) Journal of Biological Chemistry 277(19):17032-17040; Frank, R. N. (2002) American Journal of Ophthalmology133(5):693-698; Parekh, D. et. al. (2000) EMBO Journal 19(4):496-503; Newton, A. C. (2001) Chem. Rev. 101:2353-2364.

Further Definitions

The term “anti-neoplastic agent” as used herein, unless otherwise indicated, refers to agents capable of inhibiting or preventing the growth of neoplasms, or checking the maturation and proliferation of malignant (cancer) cells. Anti-neoplastic agents contemplated in accordance with the present invention include, but are not limited to alkylating agents, including busulfan, chlorambucil, cyclophosphamide, iphosphamide, melphalan, nitrogen mustard, streptozocin, thiotepa, uracil nitrogen mustard, triethylenemelamine, temozolomide, and SARCnu; antibiotics and plant alkaloids including actinomycin-D, bleomycin, cryptophycins, daunorubicin, doxorubicin, idarubicin, irinotecan, L-asparaginase, mitomycin-C, mitramycin, navelbine, paclitaxel, docetaxel, topotecan, vinblastine, vincristine, VM-26, and VP-16-213; hormones and steroids including 5α-reductase inhibitor, aminoglutethimide, anastrozole, bicalutamide, chlorotrianisene, DES, dromostanolone, estramustine, ethinyl estradiol, flutamide, fluoxymesterone, goserelin, hydroxyprogesterone, letrozole, leuprolide, medroxyprogesterone acetate, megestrol acetate, methyl prednisolone, methyltestosterone, mitotane, nilutamide, prednisolone, SERM3, tamoxifen, testolactone, testosterone, tramicnolone, and zoladex; synthetics including all-trans retinoic acid, BCNU (carmustine), CBDCA carboplatin (paraplatin), CCNU (lomustine), cis-diaminedichloroplatinum (cisplatin), dacarbazine, gliadel, hexamethylmelamine, hydroxyurea, levamisole, mitoxantrone, o, p′-DDD (lysodren, mitotane), oxaliplatin, porfimer sodium, procarbazine, GleeVec; antimetabolites including chlorodeoxyadenosine, cytosine arabinoside, 2′-deoxycoformycin, fludarabine phosphate, 5-fluorouracil, 5-FUDR, gemcitabine, camptothecin, 6-mercaptopurine, methotrexate, MTA, and thioguanine; and biologics including alpha interferon, BCG, G-CSF, GM-CSF, interleukin-2, herceptin; and the like.

The term “cancer” as used herein refers to disorders such as solid tumor cancer including colon cancer, breast cancer, lung cancer and prostrate cancer, tumor invasion, tumor growth tumor metastasis, cancers of the oral cavity and pharynx (lip, tongue, mouth, pharynx), esophagus, stomach, small intestine, large intestine, rectum, liver and biliary passages, pancreas, larynx, bone, connective tissue, skin, cervix uteri, corpus endometrium, ovary, testis, bladder, kidney and other urinary tissues, eye, brain and central nervous system, thyroid and other endocrine gland, Hodgkin's disease, non-Hodgkin's lymphomas, multiple myeloma and hematopoietic malignancies including leukemias and lymphomas including lymphocytic, granulocytic and monocytic.

Additional types of cancers which may be treated by the present invention include but are not limited to:

adrenocarcinoma, angiosarcoma, astrocytoma, acoustic neuroma, anaplastic astrocytoma, basal cell carcinoma, blastoglioma, chondrosarcoma, choriocarcinoma, chordoma, craniopharyngioma, cutaneous melanoma, cystadenocarcinoma, endotheliosarcoma, embryonal carcinoma, ependymoma, Ewing's tumor, epithelial carcinoma, fibrosarcoma, gastric cancer, genitourinary tract cancers, glioblastoma multiforme, head and neck cancer, hemangioblastoma, hepatocellular carcinoma, hepatoma, Kaposi's sarcoma, large cell carcinoma, cancer of the larynx, leiomyosarcoma, leukemias, liposarcoma, lymphatic system cancer, lymphomas, lymphangiosarcoma, lymphangioendotheliosarcoma, medullary thyroid carcinoma, medulloblastoma, meningioma mesothelioma, myelomas, myxosarcoma neuroblastoma, neurofibrosarcoma, oligodendroglioma, osteogenic sarcoma, epithelial ovarian cancer, papillary carcinoma, papillary adenocarcinomas, parathyroid tumors, pheochromocytoma, pinealoma, plasmacytomas, retinoblastoma, rhabdomyosarcoma, sebaceous gland carcinoma, seminoma, skin cancers, melanoma, small cell lung carcinoma, squamous cell carcinoma, sweat gland carcinoma, synovioma, thyroid cancer, uveal melanoma, stomach cancers, and Wilm's tumor.

The terms “enhance” or “enhancing”, as used herein, unless otherwise indicated, means to increase or prolong either in potency or duration a desired effect. Thus, in regard to “enhancing the effect of DNA-damaging agents,” the term “enhancing” refers to the ability to increase or prolong, either in potency or duration, the effect of DNA-damaging agents on a system (e.g., a tumor cell). An “enhancing-effective amount,” as used herein, refers to an amount adequate to enhance the effect of a DNA-damaging agent in a desired system (including, by way of example only, a tumor cell in a patient). When used in a patient, amounts effective for this use will depend on the severity and course of the proliferative disorder (including, but not limited to, cancer), previous therapy, the patient's health status and response to the drugs, and the judgment of the treating physician. It is considered well within the skill of the art for one to determine such enhancing-effective amounts by routine experimentation.

An “excipient” generally refers to substance, often an inert substance, added to a pharmacological composition or otherwise used as a vehicle to further facilitate administration of a compound. Examples of excipients include but are not limited to calcium carbonate, calcium phosphate, various sugars and types of starch, cellulose derivatives, gelatin, vegetable oils and polyethylene glycols.

“Eye diseases” as used herein refers to disorders such as aberrant angiogenesis, ocular angiogenesis, ocular inflammation, keratoconus, Sjogren's syndrome, myopia, ocular tumors, corneal graft rejection, corneal injury, neovascular glaucoma, corneal ulceration, corneal scarring, macular degeneration (including “Age Related Macular Degeneration (ARMD) including both wet and dry forms), proliferative vitreoretinopathy and retinopathy of prematurity.

The term “in combination with” means that the compound of Formula (I) may be administered shortly before, shortly after, concurrently, or any combination of before, after, or concurrently, with other anti-neoplasm therapies. Thus, the compound and the anti-neoplastic agent may be administered simultaneously as either as a single composition or as two separate compositions or sequentially as two separate compositions. Likewise, the compound and radiation therapy may be administered simultaneously, separately or sequentially. The compound may be administered in combination with more than one anti-neoplasm therapy. In a preferred embodiment, the compound may be administered from 2 weeks to 1 day before any chemotherapy, or 2 weeks to 1 day before any radiation therapy. In another preferred embodiment, the CHK-1 inhibitor may be administered during anti-neoplastic chemotherapies and radiation therapies. If administered following such chemotherapy or radiation therapy, the CHK-1 inhibitor may be given within 1 to 14 days following the primary treatments. The CHK-1 inhibitor may also be administered chronically or semi-chronically, over a period of from about 2 weeks to about 5 years. One skilled in the art will recognize that the amount of CHK-1 inhibitor to be administered in accordance with the present invention in combination with other antineoplastic agents or therapies is that amount sufficient to enhance the anti-neoplasm effects of anti-neoplastic agents or radiation therapies or that amount sufficient to induce apoptosis or cell death along with the anti-neoplastic or radiation therapy and/or to maintain an anti-angiogenic effect. Such amount may vary, among other factors, depending upon the size and the type of neoplasia, the concentration of the compound in the therapeutic formulation, the specific anti-neoplasm agents used, the timing of the administration of the CHK-1 inhibitors relative to the other therapies, and the age, size and condition of the patient.

The term “neoplasm” as used herein, unless otherwise indicated, is defined as in Stedman's Medical Dictionary 25th Edition (1990)and refers to an abnormal tissue that grows by cellular proliferation more rapidly than normal and continues to grow after the stimuli that initiated the new growth ceases. Neoplasms show partial or complete lack of structural organization and functional coordination compared with normal tissue, and usually form a distinct mass of tissue that may be either benign (benign tumor) or malignant (cancer).

The term “neoplasia” as used herein, unless otherwise indicated, refers to abnormal growth of cells which often results in the invasion of normal tissues, e. g., primary tumors or the spread to distant organs, e. g., metastasis. The treatment of any neoplasia by conventional non-surgical anti-neoplasm therapies may be enhanced by the present invention. Such neoplastic growth includes but not limited to primary tumors, primary tumors that are incompletely removed by surgical techniques, primary tumors which have been adequately treated but which are at high risk to develop a metastatic disease subsequently, and an established metastatic disease.

“A pharmaceutically acceptable salt” is intended to mean a salt that retains the biological effectiveness of the free acids and bases of the specified compound and that is not biologically or otherwise undesirable. A compound of the invention may possess a sufficiently acidic, a sufficiently basic, or both functional groups, and accordingly react with any of a number of inorganic or organic bases, and inorganic and organic acids, to form a pharmaceutically acceptable salt. Exemplary pharmaceutically acceptable salts include those salts prepared by reaction of the compounds of the present invention with a mineral or organic acid or an inorganic base, such as salts including sulfates, pyrosulfates, bisulfates, sulfites, bisulfites, phosphates, monohydrogenphosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, γ-hydroxybutyrates, glycolates, tartrates, methane-sulfonates, propanesulfonates, naphthalene-1-sulfonates, naphthalene-2-sulfonates, and mandelates.

If the compound of the invention is a base, the desired pharmaceutically acceptable salt may be prepared by any suitable method available in the art, for example, treatment of the free base with an inorganic acid, such as hydrochloric acid, hydrobromic acid, sulfuric acid, sulfamic acid, nitric acid, phosphoric acid and the like, or with an organic acid, such as acetic acid, phenylacetic acid, propionic acid, stearic acid, lactic acid, ascorbic acid, maleic acid, hydroxymaleic acid, isethionic acid, succinic acid, mandelic acid, fumaric acid, malonic acid, pyruvic acid, oxalic acid, glycolic acid, salicylic acid, a pyranosidyl acid, such as glucuronic acid or galacturonic acid, an alpha-hydroxy acid, such as citric acid or tartaric acid, an amino acid, such as aspartic acid or glutamic acid, an aromatic acid, such as benzoic acid, 2-acetoxybenzoic acid or cinnamic acid, a sulfonic acid, such as p-toluenesulfonic acid, methanesulfonic acid or ethanesulfonic acid, or the like.

If the compound of the invention is an acid, the desired pharmaceutically acceptable salt may be prepared by any suitable method, for example, treatment of the free acid with an inorganic or organic base, such as an amine (primary, secondary or tertiary), an alkali metal hydroxide or alkaline earth metal hydroxide, or the like. Illustrative examples of suitable salts include organic salts derived from amino acids, such as glycine and arginine, ammonia, carbonates, bicarbonates, primary, secondary, and tertiary amines, and cyclic amines, such as benzylamines, pyrrolidines, piperidine, morpholine and piperazine, and inorganic salts derived from sodium, calcium, potassium, magnesium, manganese, iron, copper, zinc, aluminum and lithium.

A “pharmacological composition” refers to a mixture of one or more of the compounds described herein, or physiologically acceptable salts thereof, with other chemical components, such as physiologically acceptable carriers and/or excipients. The purpose of a pharmacological composition is to facilitate administration of a compound to an organism.

A “physiologically acceptable carrier” refers to a carrier or diluent that does not cause significant or otherwise unacceptable irritation to an organism and does not unacceptably abrogate the biological activity and properties of the administered compound.

The term “prodrug” means compounds that are drug precursors, which following administration, release the drug in vivo via some chemical or physiological process (e.g., a prodrug on being brought to the physiological pH is converted to the desired drug form).

Prodrugs include compounds wherein an amino acid residue, or a polypeptide chain of two or more (e.g., two, three or four) amino acid residues is covalently joined through an amide or ester bond to a free amino, hydroxy or carboxylic acid group of compounds of formula (I). The amino acid residues include but are not limited to the 20 naturally occurring amino acids commonly designated by three letter symbols and also includes 4-hydroxyproline, hydroxylysine, demosine, isodemosine, 3-methylhistidine, norvalin, beta-alanine, gamma-aminobutyric acid, citrulline homocysteine, homoserine, ornithine and methionine sulfone. Additional types of prodrugs are also encompassed. For instance, free carboxyl groups can be derivatized as amides or alkyl esters. Free hydroxy groups may be derivatized using groups including but not limited to hemisuccinates, phosphate esters, dimethylaminoacetates, and phosphoryloxymethyloxycarbonyls, as outlined in Advanced Drug Delivery Reviews, 1996, 19, 115. Carbamate prodrugs of hydroxy and amino groups are also included, as are carbonate prodrugs, sulfonate esters and sulfate esters of hydroxy groups. Derivatization of hydroxy groups as (acyloxy)methyl and (acyloxy)ethyl ethers wherein the acyl group may be an alkyl ester, optionally substituted with groups including but not limited to ether, amine and carboxylic acid functionalities, or where the acyl group is an amino acid ester as described above, are also encompassed. Prodrugs thus include the use of protecting groups on the resorcinol or resorcinol-like moiety of compounds having the structure of Formula (I) which will hydrolyze under physiological conditions to give back the resorcinol or resorcinol-like moiety. Prodrugs of this type are described in J. Med. Chem. 1996, 39, 10. Free amines can also be derivatized as amides, sulfonamides or phosphonamides. All of these prodrug moieties may incorporate groups including but not limited to ether, amine and carboxylic acid functionalities.

“A pharmaceutically acceptable prodrug” is a compound that may be converted under physiological conditions or by solvolysis to the specified compound or to a pharmaceutically acceptable salt of such compound. “A pharmaceutically active metabolite” is intended to mean a pharmacologically active product produced through metabolism in the body of a specified compound or salt thereof. Prodrugs and active metabolites of a compound may be identified using routine techniques known in the art. See, e.g., Bertolini et al., J. Med. Chem., 40, 2011-2016 (1997); Shan et al., J. Pharm. Sci., 86 (7), 765-767; Bagshawe, Drug Dev. Res., 34, 220-230 (1995); Bodor, Advances in Drug Res., 13, 224-331 (1984); Bundgaard, Design of Prodrugs (Elsevier Press 1985); and Larsen, Design and Application of Prodrugs, Drug Design and Development (Krogsgaard-Larsen et al., eds., Harwood Academic Publishers, 1991).

The term “treating”, as used herein, unless otherwise indicated, means reversing, alleviating, inhibiting the progress of, or preventing the disorder or condition to which such term applies, or one or more symptoms of such disorder or condition. The term “treatment”, as used herein, unless otherwise indicated, refers to the act of treating as “treating” is defined immediately above.

Compositions comprising the compound(s)described herein can be administered for prophylactic and/or therapeutic treatments. In therapeutic applications, the compositions are administered to a patient already suffering from a proliferative disorder or condition (including, but not limited to, cancer), as described above, in an amount sufficient to cure or at least partially arrest the symptoms of the proliferative disorder or condition. An amount adequate to accomplish this is defined as “therapeutically effective amount or dose.” Amounts effective for this use will depend on the severity and course of the proliferative disorder or condition, previous therapy, the patient's health status and response to the drugs, and the judgment of the treating physician. In prophylactic applications, compositions containing the compounds described herein are administered to a patient susceptible to or otherwise at risk of a particular proliferative disorder or condition. Such an amount is defined to be a “prophylactically effective amount or dose.” In this use, the precise amounts also depend on the patient's state of health, weight, and the like. It is considered well within the skill of the art for one to determine such therapeutically effective or prophylactically effective amounts by routine experimentation (e.g., a dose escalation clinical trial).

Once improvement of the patient's conditions has occurred, a maintenance dose is administered if necessary. Subsequently, the dosage or the frequency of administration, or both, can be reduced, as a function of the symptoms, to a level at which the improved proliferative disorder or condition is retained. When the symptoms have been alleviated to the desired level, treatment can cease. Patients can, however, require intermittent treatment on a long-term basis upon any recurrence of the disease symptoms.

The amount and frequency of administration of the compounds used in the methods described herein and, if applicable, other agents will be regulated according to the judgment of the attending clinician (physician) considering such factors as age, condition and size of the patient as well as severity of the disease being treated.

The amount of the active compound administered (e.g., for treatment, prophylactic, and/or maintenance) will be dependent on the subject being treated, the severity of the disorder or condition, the rate of administration, the disposition of the compound and the discretion of the prescribing physician. However, an effective dosage is in the range of about 0.001 to about 100 mg per kg body weight per day, preferably about 1 to about 35 mg/kg/day, in single or divided doses. For a 70 kg human, this would amount to about 0.05 to about 7 g/day, preferably about 0.2 to about 2.5 g/day. In some instances, dosage levels below the lower limit of the aforesaid range may be more than adequate, while in other cases still larger doses may be employed without causing any harmful side effect, provided that such larger doses are first divided into several small doses for administration throughout the day.

Pharmaceutical compositions according to the invention may, alternatively or in addition to a compound of Formula (I), comprise as an active ingredient pharmaceutically acceptable prodrugs, pharmaceutically active metabolites, and pharmaceutically acceptable salts of such compounds and metabolites. Such compounds, prodrugs, multimers, salts, and metabolites are sometimes referred to herein collectively as “active agents” or “agents.”

### DETAILED DESCRIPTION OF THE INVENTION

According to this invention, compounds of formula I can be prepared by the reaction schemes depicted below:

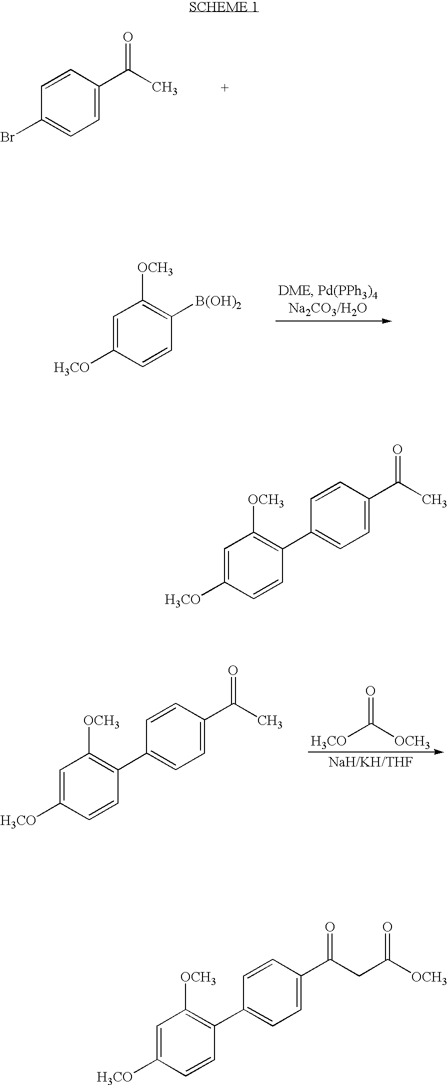

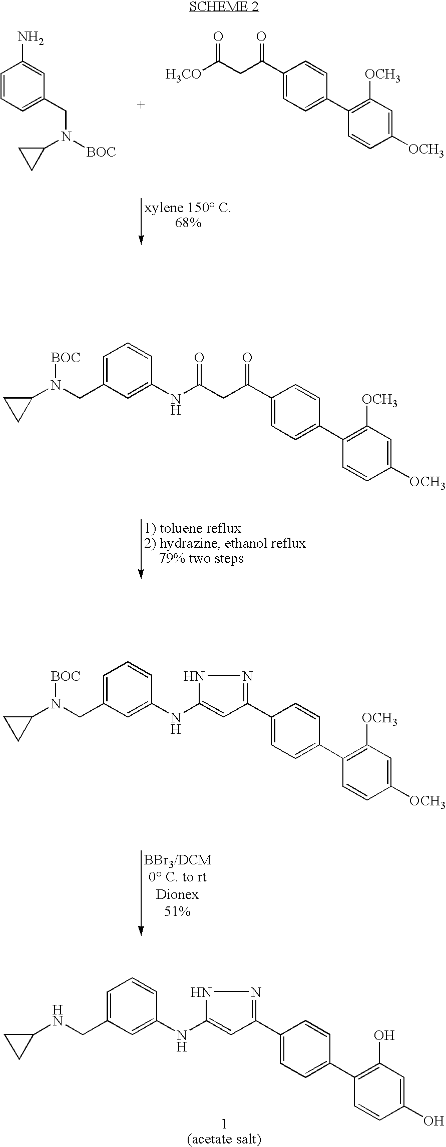

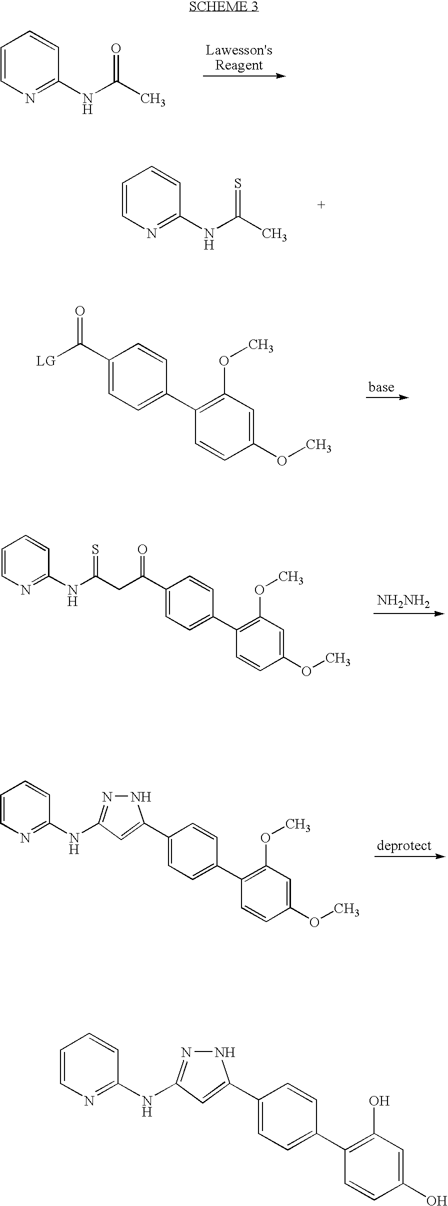

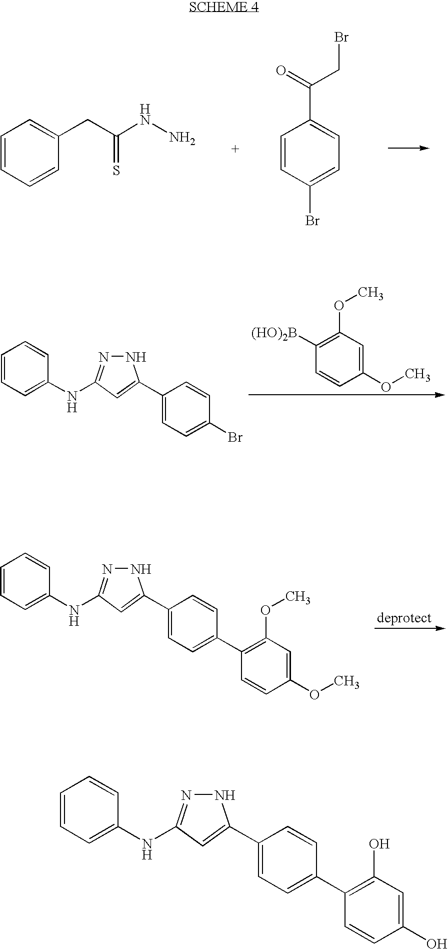

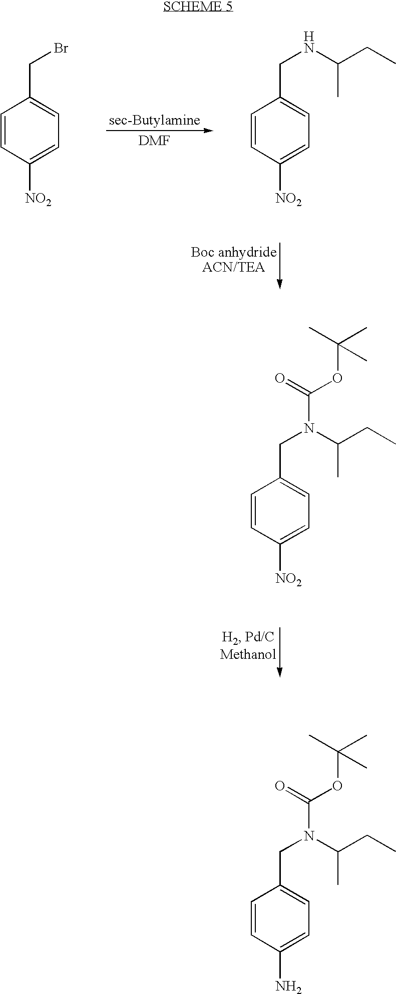

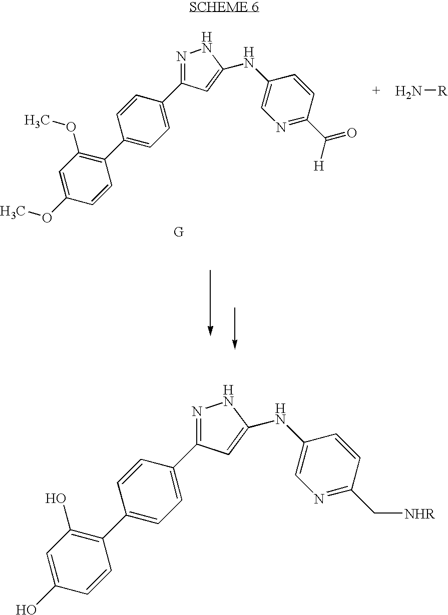

Scheme 1 depicts a synthetic scheme for two carbon-carbon bond forming reactions used to construct intermediates for compounds of the present invention from readily available starting materials. The first step in FIG. 1 depicts a transition metal-catalyzed coupling of a carbon electrophile (an aryl halide) with a carbon nucleophile (an arylboronic acid) to form a new aryl-aryl bond. The first reaction in FIG. 1 is an example of a Suzuki coupling, a versatile reaction which in principle allows the coupling of virtually any complimentary carbon electrophile and carbon nucleophile pair. The second step in Figure I depicts a base-catalyzed coupling of a carbon nucleophile (an enolate formed from the acetophenone shown) to a carbon electrophile (dimethylcarbonate). Standard work-up yields the 1,3-ketoester shown. The skilled artisan will recognize that the two coupling steps depicted in FIG. 1 may be performed in the reverse order. Solvents shown are by way of example only.

Scheme 2 depicts a synthetic scheme having three further synthetic steps used to prepare compounds described herein. The first step in FIG. 2 depicts the coupling of a nitrogen nucleophile (in this case a boc-protected aniline derivative) to a carbon electrophile (in this case, the 1,3 ketoester carbonyl carbon). A new amide bond results in this case as depicted. This versatile reaction allows the coupling of numerous pyrazolyl side chains in the final product. The second step in FIG. 2 depicts the formation of the pyrazolyl moiety from a diketo precursor using hydrazine as the heteroatom source. This reaction is carried out in two steps as indicated. While only one pyrazolyl tautomer is depicted in FIG. 2, the present method is able to produce both tautomeric forms of pyrazole moieties. The final step in FIG. 2 depicts the deprotection of the boc protected amine followed by column chromatography. Solvents and temperatures shown are by way of example only.

Scheme 3 depicts a synthetic scheme for a four step conversion of a pyridylacetamide to a pyazole product. The first step shows the conversion of an acetamide to a thioacetamide. Lawesson's reagent (1,3,2,4-dithiadiphosphetane-2,4-disulfide) converts ketonic into thioketonic groups. The second step in FIG. 3 depicts the coupling of a carbon electrophile (in this case an enthiolate generated in situ) with a carbon electrophile (LG signifies leaving group). The third step in FIG. 3 depicts the formation of the pyrazolyl moiety from a diketo precursor using hydrazine as the heteroatom source. The fourth step shows the deprotection of the phenyl hydroxides.

Scheme 4 depicts a synthetic scheme for an alternative method of synthesizing pyrazole compounds of the present invention. In the first step, a semicarbazide nucleophile reacts with the electrophilic carbonyl carbon of a 2-bromoacetophenone. After refluxing under acid conditions, the intermediate pyrazole amine (in the case shown bearing bromo substituent) is isolated. A second aryl moiety may be coupled to the aryl bromide using Suzuki coupling. Deprotection of the dimethoxy arene and column chromatography gives the desired compounds.

Scheme 5 depicts a synthetic scheme for a general method for synthesizing aryl amines useful for coupling to carbon electrophiles as depicted in FIG. 2. In a first step, a commercially available tolylbromide electrophile is treated with an amine nucleophile. In a second step, the secondary amine is boc-protected. In a third step, the arylnitrogroup is reduced to the corresponding amine. Solvents and reagents shown are by way of example only.

Scheme 6 depicts a synthetic scheme for a general method for synthesizing pyrazole compounds of the present invention. The first step depicts the reaction of a protected pyrazole compound G bearing carbonyl electrophile with a suitable primary amine. Reductive amination with sodium triacetoxyborohydride under nitrogen affords the protected amine. Deprotection of the aryl methoxy group affords the desired compound. General Synthetic Methodology

General Synthetic Methodolopy

In the examples described below, unless otherwise indicated all temperatures are set forth in degrees Celsius and all parts and percentages are by weight. Reagents were purchased from commercial suppliers such as Aldrich Chemical Company or Lancaster Synthesis Ltd. and were used without further purification unless otherwise indicated. Tetrahydrofuran (THF), N,N-dimethylformamide (DMF), dichloromethane, toluene, and dioxane were purchased from Aldrich in Sure seal bottles and used as received. All solvents were purified using standard methods readily known to those skilled in the art, unless otherwise indicated.

The reactions set forth below were done generally under a positive pressure of argon or nitrogen or with a drying tube, at ambient temperature (unless otherwise stated), in anhydrous solvents, and the reaction flasks were fitted with rubber septa for the introduction of substrates and reagents via syringe. Glassware was oven dried and/or heat dried. Analytical thin layer chromatography (TLC) was performed on glass-backed silica gel 60 F 254 plates Analtech (0.25 mm) and eluted with the appropriate solvent ratios (v/v), and are denoted where appropriate. The reactions were assayed by TLC and terminated as judged by the consumption of starting material.

Visualization of the TLC plates was done with a p-anisaldehyde spray reagent or phosphomolybdic acid reagent (Aldrich Chemical 20 wt % in ethanol) and activated with heat. Work-ups were typically done by doubling the reaction volume with the reaction solvent or extraction solvent and then washing with the indicated aqueous solutions using 25% by volume of the extraction volume unless otherwise indicated. Product solutions were dried over anhydrous Na₂SO₄ prior to filtration and evaporation of the solvents under reduced pressure on a rotary evaporator and noted as solvents removed in vacuo. Flash column chromatography (Still et al., J. Org. Chem., 43, 2923 (1978)) was done using Baker grade flash silica gel (47-61 μm) and a silica gel: crude material ratio of about 20:1 to 50:1 unless otherwise stated. Hydrogenolysis was done at the pressure indicated in the examples or at ambient pressure.

¹H-NMR spectra were recorded on a Bruker instrument operating at 300 MHz. NMR spectra were obtained as CDCl₃ solutions (reported in ppm), using chloroform as the reference standard (7.25 ppm and 77.00 ppm) or CD₃OD (3.4 and 4.8 ppm and 49.3 ppm), or internally tetramethylsilane (0.00 ppm) when appropriate. Other NMR solvents were used as needed. When peak multiplicities are reported, the following abbreviations are used: s (singlet), d (doublet), t (triplet), m (multiplet), br (broadened), dd (doublet of doublets), dt (doublet of triplets). Coupling constants, when given, are reported in Hertz (Hz).

Where HPLC chromatography is referred to in the preparations and examples below, the general conditions used, unless otherwise indicated, are as follows. The column used is a ZORBAX™ RXC18 column (manufactured by Hewlett Packard) of 150 mm distance and 4.6 mm interior diameter. The samples are run on a Hewlett Packard-1100 systemA gradient solvent method is used running 100 percent ammonium acetate/acetic acid buffer (0.2 M) to 100 percent acetonitrile over 10 minutes. The system then proceeds on a wash cycle with 100 percent acetonitrile for 1.5 minutes and then 100 percent buffer solution for 3 minutes. The flow rate over this period is a constant 3 ml/minute.

Those compounds of Formula (I) that are acidic in nature are capable of forming base salts with various pharmacologically acceptable cations. These salts can easily be prepared by treating the corresponding acidic compounds with an aqueous solution containing the desired pharmacologically acceptable cations, and then evaporating the resulting solution to dryness, preferably under reduced pressure. Alternatively, they may also be prepared by mixing lower alkanolic solutions of the acidic compounds and the desired alkali metal alkoxide together, and then evaporating the resulting solution to dryness in the same manner as before. In either case, stoichiometric quantities of reagents are preferably employed in order to ensure completeness of reaction and maximum yields of the desired final product.

Certain compounds of Formula (I) may have asymmetric centers and therefore exist in different enantiomeric forms. All optical isomers and stereoisomers of the compounds of Formula (I), and mixtures thereof, are considered to be fully described herein. With respect to the compounds of Formula (I), also fully described herein are the use of a racemate, one or more enantiomeric forms, one or more diastereomeric forms, or mixtures thereof.

The compounds of Formula (I) may also exist as tautomers. For example, compounds T and T′ shown below are tautomers related by the site of protonation of inequivalent nitrogens. Such tautomers may be distinguished by X-ray crystallography (single crystal and powder diffraction), and spectroscopic methods, for example IR spectroscopy. Such tautomers may be distinguished in solution and solid state NMR methods although if proton exchange between tautomers is rapid, only a single signal may be observed in solution. Both tautomers of the compounds of Formula (I) are considered to be fully described herein. The compositions and methods described herein include the use of all such tautomers and mixtures thereof.

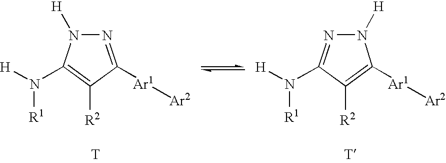

The compounds described herein, including the pharmaceutically acceptable prodrugs, pharmaceutically active metabolites, and pharmaceutically acceptable salts of such compounds, also include isotopically-labelled compounds, which are identical to those recited in Formula (I), but for the fact that one or more atoms are replaced by an atom having an atomic mass or mass number different from the atomic mass or mass number usually found in nature. Examples of isotopes that can be incorporated into compounds disclosed herein include, but are not limited to: isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively. Certain isotopically-labelled compounds, for example those into which radioactive isotopes such as ³H and ¹⁴C are incorporated, are useful in drug and/or substrate tissue distribution assays. By way of example only, tritiated, i.e., ³H, and carbon-14, i.e., ¹⁴C, isotopes are preferred for their ease of preparation and detectability. Further, substitution with heavier isotopes, including by way of example only, deuterium, i.e., ²H, can afford certain therapeutic advantages resulting from greater metabolic stability, for example increased in vivo half-life or reduced dosage requirements and, hence, may be preferred in some circumstances. Isotopically labelled compounds, including by way of example only, compounds of Formula (I) (as well as metabolites, prodrugs, and pharmaceutically acceptable salts thereof) can generally be prepared by carrying out the procedures disclosed in the Figures and/or in the Examples and Preparations below, by substituting an isotopically labelled reagent for a non-isotopically labeled reagent.

In the case of agents that are solids, it is understood by those skilled in the art that the inventive compounds and salts may exist in different crystal or polymorphic forms, all of which are intended to be within the scope of the present disclosure and specified formulas.

The examples and preparations provided below further illustrate and exemplify the aminopyrazole compounds described herein and methods of preparing such compounds. It is to be understood that the scope of the present disclosure is not limited in any way by the scope of the following examples and preparations. In the following examples, “Ac” means acetyl, “Et” means ethyl, “Me” means methyl, and “Bu” means butyl.

Synthetic Methods

The synthetic schemes shown in FIGS. 1 to 6 were used for the preparation of compounds presented herein. The skilled artisan will recognize that alternative synthetic methodology may be used to prepare the same compound. Additional data for the compounds described herein may be found in Table I. A more detailed description for the synthesis of certain exemplary compounds of the present invention is provided in the “Examples” section below. The terms “intermediate” and “compound” are used interchangeably. U.S. Pat. No. 4,803,216 describes the syntheses of some pyrazole-3-amines.

Figure I features a carbon-carbon coupling reaction which forms the linear biphenyl portion characteristic of many examples disclosed herein. This versatile reaction finds broad use in organic synthesis and typically couples organoboranes or boronate (or organostannane) moieties with an aryl, vinyl, or acetylenic halides, sulfonates, or acetates. Such reagents do not ordinarily react at any appreciable rate, but readily do so in the presence of a catalyst, for example, in the presence a low valent transition metal complexes, preferred transition metal complexes being palladium complexes wherein the palladium has a formal oxidation state of zero (0) or two (II). Other ligating groups associated with the transition metal may also be present, e. g., phosphines, phosphonates, arsines, and other equivalents known to the art; these ligands serve chiefly to prevent the nucleation of Pd atoms into palladium metal.

Co-catalysts such as Cul are also often present in such coupling reactions. For a general description of the coupling of carbon electrophiles and nucleophiles, see Comprehensive Organic Synthesis, Trost et al., Pergamon Press, Chapter 2.4: Coupling Reactions Between sp² and sp Carbon Centers, pp 521-549, and pp 950-953, hereby incorporated by reference.

The palladium-catalyzed coupling of organoboranes (E=B above) with carbon electrophiles to yields a new carbon-carbon bond and is known as a Suzuki coupling [Suzuki et al. J. Am. Chem. Soc. 1989, 111,314]. The palladium-catalyzed coupling of organostannane reagents (E=Sn in figure above) and carbon electrophiles is known as a Stille coupling reaction [See Stille, J. K. Angew. Chem. Int. Ed. Engl. 1986, 25, 508 and Farina & Roth, Adv. Met.-Org. Chem. 1996,5, 1-53.

Pharmaceutical Compositions/Formulations, Dosaging, and Modes of Administration

Methods of preparing various pharmaceutical compositions with a specific amount of active compound are known, or will be apparent, to those skilled in this art. In addition, those of ordinary skill in the art are familiar with formulation and administration techniques. Such topics would be discussed, e.g., in Goodman and Gilman's The Pharmacological Basis of Therapeutics, current edition, Pergamon Press; and Remington's Pharmaceutical Sciences (current edition.) Mack Publishing Co., Easton, Pa. These techniques can be employed in appropriate aspects and embodiments of the methods and compositions described herein. The following examples are provided for illustrative purposes only and are not meant to serve as limitations of the present disclosure.

The compounds utilized in the methods described herein may be administered either alone or in combination with pharmaceutically acceptable carriers, excipients or diluents, in a pharmaceutical composition, according to standard pharmaceutical practice.

Administration of the compounds described herein (hereinafter the “active compound(s)”) can be effected by any method that enables delivery of the compounds to the site of action. These methods include oral routes, intraduodenal routes, parenteral injection (including intravenous, subcutaneous, intramuscular, intravascular or infusion), topical, and rectal administration. For example, the therapeutic or pharmaceutical compositions described herein can be administered locally to the area in need of treatment. This may be achieved by, for example, but not limited to, local infusion during surgery, topical application, e.g., cream, ointment, injection, catheter, or implant, said implant made, e.g., out of a porous, non-porous, or gelatinous material, including membranes, such as sialastic membranes, or fibers. The administration can also be by direct injection at the site (or former site) of a tumor or neoplastic or pre-neoplastic tissue.

Still further, the therapeutic or pharmaceutical composition can be delivered in a vesicle, e.g., a liposome (see, for example, Langer, Science, 249:1527-1533 (1990); Treat et al., 1989, Liposomes in the Therapy of Infectious Disease and Cancer, Lopez-Bernstein and Fidler (eds.), Liss, N.Y., pp. 353-365). The preparation and characterization of liposomes as therapeutic delivery systems has been reviewed. See Vemuri and Rhodes, Pharmaceutical Acta Helvetiae, 70, 95-111, (1995).

The pharmaceutical compositions used in the methods described herein can be delivered in a controlled release system. In one embodiment, a pump may be used (see, Sefton, 1987, CRC Crit. Ref. Biomed. Eng. 14:201; Buchwald et al., 1980, Surgery, 88:507; Saudek et al., 1989, N. Engl. J. Med., 321:574). Additionally, a controlled release system can be placed in proximity of the therapeutic target (see, Goodson, 1984, Medical Applications of Controlled Release, Vol. 2, pp. 115-138).

The pharmaceutical compositions used in the methods or compositions described herein can contain the active ingredient in a form suitable for oral use, for example, as tablets, troches, dragee cores, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients, which are suitable for the manufacture of tablets. These excipients may be, for example, inert diluents, such as calcium carbonate, sodium carbonate, lactose, calcium phosphate or sodium phosphate; granulating and disintegrating agents, such as microcrystalline cellulose, sodium crosscarmellose, corn starch, or alginic acid; binding agents, for example starch, gelatin, polyvinylpyrrolidone or acacia, and lubricating agents, for example, magnesium stearate, stearic acid or talc. The tablets may be uncoated or they may be coated by known techniques to mask the taste of the drug or delay disintegration and absorption in the gastrointestinal tract and thereby provide a sustained action over a longer period. For example, a water soluble taste masking material such as hydroxypropylmethyl-cellulose or hydroxypropylcellulose, or a time delay material such as ethyl cellulose, or cellulose acetate butyrate may be employed.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water soluble carrier such as polyethyleneglycol or an oil medium, for example peanut oil, liquid paraffin, or olive oil.

Aqueous suspensions can contain the active material in admixture with excipients suitable for the manufacture of aqueous suspensions. Such excipients can act as suspending agents and include, e.g., sodium carboxymethylcellulose, methylcellulose, hydroxypropylmethyl-cellulose, sodium alginate, polyvinylpyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethylene-oxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose, saccharin or aspartame.

Oily suspensions may be formulated by suspending the active ingredient in a vegetable oil, for example arachis oil, olive oil, sesame oil or coconut oil, or in mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant, e.g., butylated hydroxyanisol, alpha-tocopherol, or ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present. These compositions may be preserved by the addition of antioxidant(s).

The pharmaceutical compositions used in the compositions and methods described herein may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring phosphatides, for example soy bean lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening, flavoring agents, preservatives and antioxidants.

Syrups and elixirs may be formulated with sweetening agents, for example glycerol, propylene glycol, sorbitol or sucrose. Such formulations may also contain a demulcent, a preservative, flavoring and coloring agents and antioxidant.

Pulmonary administration by inhalation may be accomplished by means of producing liquid or powdered aerosols, for example, by using any of various devices known in the art (see e.g. Newman, S. P., 1984, in Aerosols and the Lung, Clarke and Pavia (Eds.), Butterworths, London, England, pp. 197-224; PCT Publication No. WO 92/16192 dated Oct. 1, 1992; PCT Publication No. WO 91/08760 dated Jun. 27, 1991; NTIS Patent Application 7-504-047 filed Apr. 3, 1990 by Roosdorp and Crystal) including but not limited to nebulizers, metered dose inhalers, and powder inhalers. Various delivery devices are commercially available and can be employed, including, by way of example only: Ultravent nebulizer (Mallinckrodt, Inc, St. Louis, Mo.); Acorn II nebulizer (Marquest Medical Products, Englewood, Colo.); Ventolin metered dose inhalers (Glaxo Inc., Research Triangle Park, N.C.); Spinhaler powder inhaler (Fisons Corp., Bedford, Mass.) or Turbohaler (Astra). Such devices typically entail the use of formulations suitable for dispensing from such a device, in which a propellant material may be present.

A nebulizer may be used to produce aerosol particles, or any of various physiologically inert gases may be used as an aerosolizing agent. Other components such as physiologically acceptable surfactants (e.g. glycerides), excipients (e.g. lactose), carriers (e.g. water, alcohol), and diluents may also be included. Ultrasonic nebulizers may also be used.

As will be understood by those skilled in the art of delivering pharmaceuticals by the pulmonary route, a major criteria for the selection of a particular device for producing an aerosol is the size of the resultant aerosol particles. Smaller particles are needed if the drug particles are mainly or only intended to be delivered to the peripheral lung, i.e. the alveoli (e.g. 0.1-3 μm), while larger drug particles are needed (e.g. 3-10 μm) if delivery is only or mainly to the central pulmonary system such as the upper bronchi. Impact of particle sizes on the site of deposition within the respiratory tract is generally known to those skilled in the art.

The pharmaceutical compositions may be in the form of a sterile injectable aqueous solutions. Among the acceptable vehicles and solvents that may be employed are water, Ringers solution and isotonic sodium chloride solution.

The sterile injectable preparation may also be a sterile injectable oil-in-water microemulsion where the active ingredient is dissolved in the oily phase. For example, the active ingredient may be first dissolved in a mixture of soybean oil and lecithin. The oil solution then introduced into a water and glycerol mixture and processed to form a microemulsion.

The injectable solutions or microemulsions may be introduced into a patient's blood-stream by local bolus injection. Alternatively, it may be advantageous to administer the solution or microemulsion in such a way as to maintain a constant circulating concentration of the instant compound. In order to maintain such a constant concentration, a continuous intravenous delivery device may be utilized. Carrier formulations appropriate for intravenous administration include by way of example only, mixtures comprising water and polyethylene glycol (PEG), e.g., 50/50 w/w.

The pharmaceutical compositions may be in the form of a sterile injectable aqueous or oleagenous suspension for intramuscular and subcutaneous administration. This suspension may be formulated according to the known art using those suitable dispersing or wetting agents and suspending agents, which have been mentioned above. The sterile injectable preparation may also be a sterile injectable solution or suspension in a non-toxic parenterally-acceptable diluent or solvent, for example as a solution in 1,3-butanediol. Exemplary parenteral administration forms also include solutions or suspensions of active compounds in sterile aqueous solutions, for example, aqueous propylene glycol or dextrose solutions. All such dosage forms can be suitably buffered, if desired. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The aminopyrazoles used in the methods and compositions described herein may also be administered in the form of suppositories for rectal administration of the drug. These compositions can be prepared by mixing the aminopyrazoles described herein with a suitable non-irritating excipient, which is solid at ordinary temperatures but liquid at the rectal temperature and will therefore melt in the rectum to release the drug. Such materials include cocoa butter, glycerinated gelatin, hydrogenated vegetable oils, mixtures of polyethylene glycols of various molecular weights and fatty acid esters of polyethylene glycol.

For topical use, creams, ointments, jellies, solutions or suspensions, etc., containing at least one of the aminopyrazole compounds described herein can be used. As used herein, topical application can include mouth washes and gargles.

The compounds used in the methods and compositions described herein can be administered in intranasal form via topical use of suitable intranasal vehicles and delivery devices, or via transdermal routes, using those forms of transdermal skin patches well known to those of ordinary skill in the art. To be administered in the form of a transdermal delivery system, the dosage administration will, of course, be continuous rather than intermittent throughout the dosage regimen.

The methods and compounds described herein may also be used in conjunction with other well known therapeutic agents that are selected for their particular usefulness against the condition that is being treated. For example, the instant compounds may be useful in combination with known anti-cancer and cytotoxic agents, as described elsewhere in this disclosure.

In general, the compounds described herein and, in embodiments where combinational therapy is employed, other agents do not have to be administered in the same pharmaceutical composition, and may, because of different physical and chemical characteristics, have to be administered by different routes. The determination of the mode of administration and the advisability of administration, where possible, in the same pharmaceutical composition, is well within the knowledge of the skilled clinician. The initial administration can be made according to established protocols known in the art, and then, based upon the observed effects, the dosage, modes of administration and times of administration can be modified by the skilled clinician. The particular choice of compounds used will depend upon the diagnosis of the attending physicians and their judgment of the condition of the patient and the appropriate treatment protocol. The compounds may be administered concurrently (e.g., simultaneously, essentially simultaneously or within the same treatment protocol) or sequentially, depending upon the nature of the proliferative disease, the condition of the patient, and the actual choice of compounds used.

The determination of the order of administration, and the number of repetitions of administration of each therapeutic agent during a treatment protocol, is well within the knowledge of the skilled physician after evaluation of the disease being treated and the condition of the patient.

### Combination Therapies

Compounds of Formula (I) may be used in combination with conventional antineoplasm therapies to treat mammals, especially humans, with neoplasia. The procedures for conventional anti-neoplasm therapies, including chemotherapies using anti-neoplastic agents and therapeutic radiation, are readily available, and routinely practiced in the art, e.g., see Harrison's PRINCIPLES OF INTERNAL MEDICINE 11^{th} edition, McGraw-Hill Book Company.

The compositions and methods described herein may be used in conjunction with DNA-damaging agents to treat cell proliferative diseases and cancer. Because the compositions described herein modulate and/or inhibit the activity of CHK1, damage to DNA caused by DNA-damaging agents, may not be fully repaired by the cellular machinery if the compositions described herein are administered with (e.g., prior to, simultaneously with, or after) DNA-damaging agents. When administered with a DNA-damaging agent, the compositions described herein, there will be an increased likelihood that the mutations and damage that have occurred to the DNA are transferred to the daughter cells, or remain present in the original cell. As a result, cells should be more susceptible to the damage caused by the DNA-damaging agents, and have significantly reduced viability (e.g., increased susceptibility to apoptosis).

There are many methods known in the art for damaging the DNA of a cell and all such methods are included within the scope of the methods described herein. By way of example only, DNA-damaging agents include radiation, cytotoxic agents, antibodies, heat, agents that induce apoptosis, anti-tumor agents, chemotherapeutic agents, and other anti-proliferative agents.

The term “chemotherapeutic agent” as used herein includes, for example, hormonal agents, antimetabolites, DNA interactive agents, tubilin-interactive agents, and others such as aspariginase or hydroxyureas.

DNA-interactive agents include alkylating agents, such as cisplatin, cyclophosphamide, altretamine; DNA strand-breakage agents, such as bleomycin; intercalating topoisomerase II inhibitors, e.g., dactinomycin and doxorubicin); nonintercalating topoisomerase II inhibitors such as, etoposide and teniposide; and the DNA minor groove binder plicamydin, for example.

Alkylating agents may form covalent chemical adducts with cellular DNA, RNA, or protein molecules, or with smaller amino acids, glutathione, or similar chemicals. Examples of typical alkylating agents include, but are not limited to, nitrogen mustards, such as chlorambucil, cyclophosphamide, isofamide, mechlorethamine, melphalan, uracil mustard; aziridine such as thiotepa; methanesulfonate esters such as busulfan; nitroso ureas, such as carmustine, lomustine, streptozocin; platinum complexes, such as cisplatin, carboplatin; bioreductive alkylator, such as mitomycin, and procarbazine, dacarbazine and altretamine. DNA strand-breaking agents include bleomycin, for example.

DNA topoisomerase II inhibitors may include intercalators such as the following: amsacrine, dactinomycin, daunorubicin, doxorubicin (adriamycin), idarubicin, and mitoxantrone; as well as nonintercalators such as etoposide and teniposide.

An example of a DNA minor groove binder is plicamycin.

Antimetabolites generally interfere with the production of nucleic acids and thereby growth of cells by one of two major mechanisms. Certain drugs inhibit production of deoxyribonucleoside triphosphates that are the precursors for DNA synthesis, thus inhibiting DNA replication. Examples of these compounds are analogues of purines or pyrimidines and are incorporated in anabolic nucleotide pathways. These analogues are then substituted into DNA or RNA instead of their normal counterparts.

Antimetabolites useful as chemotherapeutic agents include, but are not limited to: folate antagonists such as methotrexate and trimetrexate; pyrimidine antagonists, such as fluorouracil, fluorodeoxyuridine, CB3717, azacitidine, cytarabine, and floxuridine; purine antagonists such as mercaptopurine, 6-thioguanine, fludarabine, pentostatin; and ribonucleotide reductase inhibitors such as hydroxyurea.

Tubulin interactive agents act by binding to specific sites on tubulin, a protein that polymerizes to form cellular microtubules. Microtubules are critical cell structure units and are required for cell division. These therapeutic agents disrupt the formation of microtubules. Exemplary tubulin-interactive agents include vincristine and vinblastine, both alkaloids and paclitaxel (Taxol).

Hormonal agents are also useful in the treatment of cancers and tumors, but only rarely in the case of B cell malignancies. They are used in hormonally susceptible tumors and are usually derived from natural sources. Hormonal agents include, but are not limited to, estrogens, conjugated estrogens and ethinyl estradiol and diethylstilbesterol, chlortrianisen and idenestrol; progestins such as hydroxyprogesterone caproate, medroxyprogesterone, and megestrol; and androgens such as testosterone, testosterone propionate; fluoxymesterone, and methyltestosterone.

Adrenal corticosteroids are derived from natural adrenal cortisol or hydrocortisone and are used to treat B cell malignancies. They are used because of their anti-inflammatory benefits as well as the ability of some to inhibit mitotic divisions and to halt DNA synthesis. These compounds include, but are not limited to, prednisone, dexamethasone, methylprednisolone, and prednisolone.

Leutinizing hormone releasing hormone agents or gonadotropin-releasing hormone antagonists are used primarily the treatment of prostate cancer. These include leuprolide acetate and goserelin acetate. They prevent the biosynthesis of steroids in the testes.

Antihormonal antigens include, for example, antiestrogenic agents such as tamoxifen, antiandrogen agents such as flutamide; and antiadrenal agents such as mitotane and aminoglutethimide.

Other agents include hydroxyurea (which appears to act primarily through inhibition of the enzyme ribonucleotide reductase), and asparaginase (an enzyme which converts asparagine to aspartic acid and thus inhibits protein synthesis).

Included within the scope of cancer therapy agents are radiolabeled antibodies, including but not limited to, Zevalin™ (IDEC Pharmaceuticals Corp.) and Bexxar™ (Corixa, Inc.); the use of any other radioisotope (e.g., ⁹⁰Y and ¹³¹I) coupled to an antibody or antibody fragment that recognizes an antigen expressed by a neoplasm; external beam radiation or any other method for administration of radiation to a patient.

Further included within the scope of cancer therapy agents are cytotoxins, including but not limited to an antibody or antibody fragment linked to a cytotoxin, or any other method for selectivly delivering a cytotoxic agent to a tumor cell.

Further included within the scope of cancer therapy agents are selective methods for destroying DNA, or any method for delivering heat to a tumor cells, including by way of example only, nanoparticles.

Further included within the scope of cancer therapy agents is the use of unlabeled antibodies or antibody fragments capable of killing or depleting tumor cells, including by way of example only, Rituxan™ (IDEC Pharmaceuticals Corp.) and Herceptin™ (Genentech).

### EXAMPLES

### Synthetic Intermediates Prepared According to Scheme I and Used in the Preparation of Compounds Described in Examples 1-16

A. Preparation of Intermediate A: 2′,4′-Dimethoxy-4-acetyl biphenyl (A)

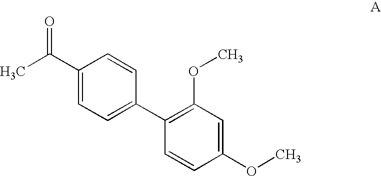

Into a solution of 4-bromoacetophenone (7 g, 35.4 mmol) in DME (135 mL), were added 2,4-dimethoxyphenyl boronic acid (8.4 g, 46.2 mmol), Pd(PPh₃)₄ (2.03 g, 1.8 mmol), and 37 mL of 2M Na₂CO₃ 1-under N₂. The mixture was refluxed with stirring for 17 hours and allowed to cool to room temperature. The reaction solution was diluted with ethyl acetate. The organic layer was separated, washed twice with water, and dried over Na₂SO₄. Solvent removal under reduced pressure gave a sticky residue. The residue was purified using silica gel column chromatography with ethyl acetate: hexanes (1:4) eluents to afford 1-(2′,4′-dimethoxy-1,1′-biphenyl-4-yl)ethanone as a white solid. Yield: 7.45 g (82%). ¹H NMR (DMSO-d₆) δ 7.95 (d, 2H), 7.58 (d, 2H), 7.28 (d, 1H), 6.65 (t, 2H), 3.81 (s, 3H), 3.77 (s, 3H), 2.58 (s, 3H)

Intermediates B and C were prepared using an analogous procedure using the corresponding aryl boronic acids. The versatility and reliability of the modified Suzuki coupling allows the coupling of a great variety of unsaturated and saturated compounds.
B. Preparation of Intermediate B: 2′,4′-Dimethoxy-5′-methyl-4-acetyl biphenyl (B)

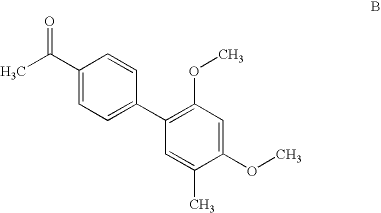

Intermediate B was prepared using a procedure analogous to that for Intermediate A. ¹H NMR (acetone-d₆) δ 7.97 (d, 2H), 7.62 (d, 2H), 7.14 (s, 1H), 6.75 (s, 1H), 3.91 (s, 3H), 3.84 (s, 3H), 2.58 (s, 3H), 2.15 (s, 3H).
C. Preparation of Intermediate C: 2′,4′-Dimethoxy-6′-methyl-4-acetyl biphenyl (C)

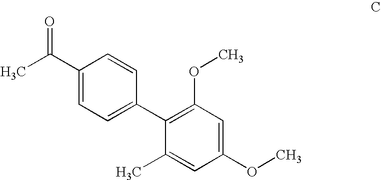

Intermediate C was prepared using a procedure analogous to that for Intermediate A. ¹H NMR (CDCl₃) δ 7.99 (d, 2H), 7.30 (d, 2H), 6.43 (s, 1H), 6.40 (s, 1H), 3.84 (s, 3H), 2.68 (s, 3H), 2.06 (s, 3H).
D. Preparation of Intermediate D: Methyl 3-(2′,4′-dimethoxy-1,1′-biphenyl-4-yl)-3-oxopropanoate (D)

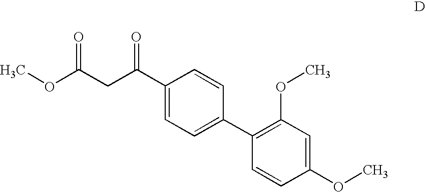

A mixture of NaH (3.48 g, 87 mmol), THF (158 mL), dimethyl carbonate (26.1 g, 290 mmol), and a small part of a solution of 1-(2′,4′-dimethoxy-1,1′-biphenyl-4-yl)ethanone in THF (29 mL) was refluxed for 15 minutes. After addition of a catalytic amount of KH, the remaining solution of 1-(2′,4′-dimethoxy-1,1′-biphenyl-4-yl)ethanone in THF was added dropwise and refluxed for at least 30 minutes and then allowed to cool to room temperature. The mixture was poured into ice water and diluted with diethyl ether and ethyl acetate. The organic layer was separated and washed with water and brine 2 times. Solvent was removed under reduced pressure. The residue was washed with hexanes and dried to afford the title compound, methyl 3-(2′,4′-dimethoxy-1,1′-biphenyl-4-yl)-3-oxopropanoate as a yellow solid in quantitative yield. ¹H NMR (DMSO-d₆) δ 7.95 (d, 2H), 7.61 (d, 2H), 7.29 (d, 1H), 6.66 (t, 2H), 4.21 (s, 2H), 3.81 (s, 3H), 3.77 (s, 3H), 3.66 (s, 3H).
E. Preparation of Intermediate E: Methyl 3-(2′,4′-dimethoxy-5′-methyl-1,1′-biphenyl-4-yl)-3-oxopropanoate (E)

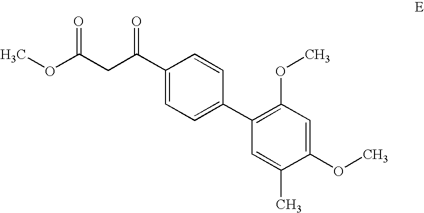

Intermediate E was prepared using an analogous procedure using the corresponding alkyl substituted biphenyls. ¹H NMR (CDCl₃) δ 7.95 (d, 2H), 7.63 (d, 2H), 7.11 (s, 1H), 6.52 (s, 1H), 4.02 (s, 2H), 3.89 (s, 3H), 3.80 (s, 3H), 3.76 (s, 3H), 2.19 (s, 3H)

F. Preparation of Intermediate F: Methyl 3-(2′,4′-dimethoxy-6′-methyl-1,1′-biphenyl-4-yl)-3-oxopropanoate (F)

Intermediate F was prepared using an analogous procedure using the corresponding alkyl substituted biphenyls.

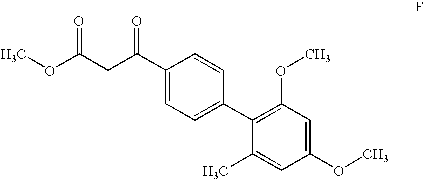

¹H NMR (CDCl₃) δ 7.98 (d, 2H), 7.33 (d, 2H), 6.44 (s, 1H), 6.41 (s, 1H), 4.04 (s, 2H), 3.84 (s, 3H), 3.78 (s, 3H), 3.68 (s, 3H), 2.06 (s, 3H).

### Example 1

### Synthesis of Compound 1

A. Preparation of Intermediate 1a: N-(3-Cyanophenyl)-3-(2′,4′-dimethoxy-1,1′-biphenyl-4-yl)-3-oxopropanamide

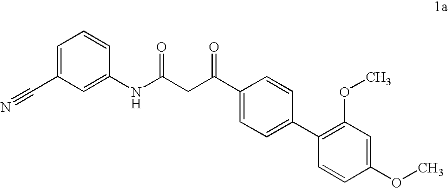

To a solution of methyl 3-(2′,4′-dimethoxy-1,1′-biphenyl-4-yl)-3-oxopropanoate(499 mg, 1.59 mmol) in xylenes (3 mL) was added a solution of 3-aminobenzonitrile(206 mg, 1.75 mmol) in xylenes (3 mL). The mixture was heated under nitrogen in a 150° C. oil bath for 6 hrs, after which time TLC indicated completion of the reaction. The amide product was purified by trituration using ethyl acetate:hexanes 1:5. The insoluble material was collected by filtration and washed several times with ethyl acetate:hexanes 1:5 followed by ethyl acetate:hexanes 1:3. This material was then vacuum pump dried overnight yielding the title compound as a yellow powder (262 mg, 41%). ¹H NMR (DMSO-d₆) δ: 3.78 (3H, s), 3.81 (3H, s), 4.19 (2H, s) 6.64 (1H, m), 6.60 (1H, t, J=2.08 Hz), 7.29 (1H, m), 7.55 (2H, m), 7.64 (2H, d, J=8.47), 7.78 (1H, m), 8.01 (1H, d, J=8.29 Hz), 8.09 (1H, m), 10.58 (1H, s).
B. Preparation of Intermediate 1b: 3-{[3-(2′,4′-Dimethoxy-1,1-biphenyl-4-yl)-1H-pyrazol-5-yl]amino}benzonitrile

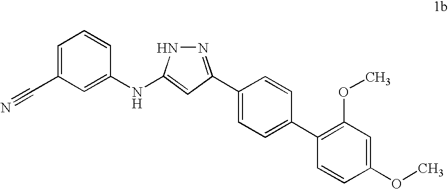

To a stirred suspension of 1b, prepared as in the previous step, in anhydrous tetrahydrofuran (15 mL) was added Lawesson's Reagent (314 mg, 0.78 mmol), under nitrogen. After heating for 3 hrs for 150° C., the reaction was concentrated to a dark amber oil. The crude material was dissolved in absolute ethanol to which was then added glacial acetic acid (0.06 mL, 0.98 mmol). The system was evacuated and flushed with nitrogen and hydrazine monohydrate (0.05 mL, 0.98 mmol) was added. The reaction was heated to reflux for 2 hours, then stirred at room temperature for 2 days. The product was purified by flash column chromatography using ethyl acetate:hexanes 1:3, 1:2, 1:1 and then methanol:chloroform 1:20 yielding the title compound as a light amber oil (146 mg, 57%). ¹H NMR (CDCl₃) δ: 3.80 (3H, s), 3.86 (3H, s), 6.56 (2H, m), 6.84 (1H, m), 7.1 (1H, m), 7.23 (2H, m), 7.60 (5H, m), 7.89 (1H, m).
C. Preparation of

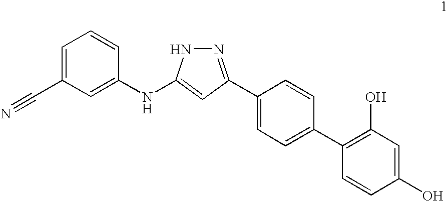

Compound 1 was synthesized using the procedures of Figure II (see also preparation of Compound 3 (Example 3C)). ¹H NMR (MeOD-d₃) δ: 6.18 (1H, s), 6.31 (2H, m), 6.99 (1H, d, J=7.58 Hz), 7.04 (1H, d, J=8.09 Hz), 7.27 (1H, d, J=7.32), 7.38 (1H, m), 7.57 (5H, bm). MS (APCI positive) 369.1.

### Example 2

### Synthesis of Compound 2

A. Preparation of Intermediate 2a: N-(4-cyanophenyl)-3-(2′,4′-dimethoxy-1,1 ′-biphenyl-4-yl)-3-oxopropanamide

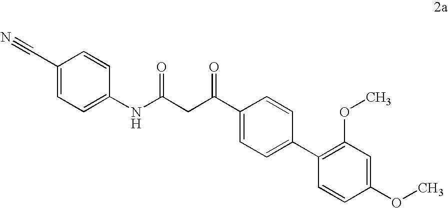

Compound 2a was synthesized using the same procedure as that of compound 1a. ¹H NMR (DMSO-d₆) δ: 3.78 (3H, s), 3.81 (3H, s), 4.21 (2H, s), 6.65 (1H, d, J=8.59), 6.69 (1H, m), 7.31 (1H, d, J=8.33), 7.64 (2H, d, J=8.59), 7.78 (5H, m), 8.00 (2H, d, J=8.34), 10.65 (1H, s).
B. Preparation of Intermediate 2b: 4-{[3-(2′,4′-dimethoxy-1,1′-biphenyl-4-yl)-1H-pyrazol-5-yl]amino}benzonitrile

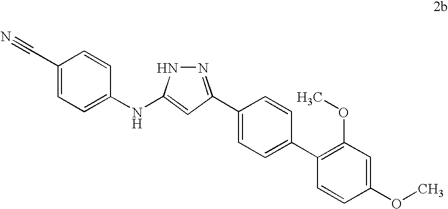

Compound 2b was synthesized using the same procedure as that of compound 1b. ¹H NMR (CDCl₃) δ: 3.81 (3H, s), 3.87 (3H, s), 6.58 (2H, m), 6.90 (1H, m), 7.21 (1H, m), 7.26 (1H, m), 7.47 (2H, bm), 7.62 (3H, bm), 7.71 (1H, d, J=8.29), 7.88 (1H, m)
C. Preparation of

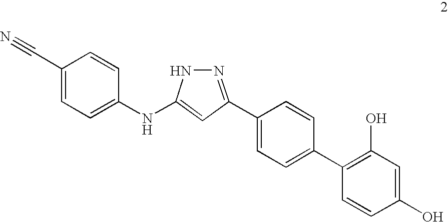

Compound 2 was synthesized using the procedures of Figures I and II (see general preparation of Compound 3). ¹H NMR (Acetone-d₆) δ: 6.38 (1H, s), 6.46 (1H, dd, J=8.33 Hz), 6.54 (1H, m), 7.20 (1H, d, J=8.34 Hz), 7.57 (5H, m), 7.66 (2H, d, J=8.59), 7.74 (2H, d, J=8.33).

### Example 3

### Preparation of Compound 3

A. Preparation of Intermediate 3a: Cyclopropyl-{3-[3-(2′,4′-dimethoxy-biphenyl-4-yl)-3-oxo-propionylamino]-benzyl}-carbamic acid tert-butyl ester

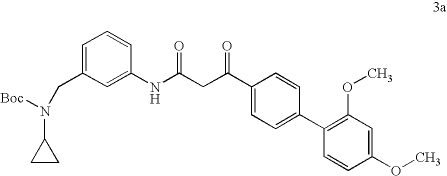

Compound 3a was synthesized using the same procedure as that of compound 1a. ¹H NMR (CDCl₃) δ 9.42 (b, 1H), 8.05 (d, 2H), 7.64 (d, 2H), 7.51 (m, 2H), 7.26 (m, 2H), 6.99 (d, 1H), 6.57 (m, 2H), 4.42 (s, 2H), 4 12 (s, 2H), 3.86 (s, 3H), 3.81 (s, 3H), 2,49 (b, 1H), 1.47 (s, 9H), 0.71 (m, 4H). MS (ESI) M⁺+1, 545.
B. Preparation of Intermediate 3b: 4′-[5-(N-Boc-3-Cyclopropylaminomethyl-phenylamino)-2H-pyrazol-3-yl]-2,4-dimethoxy biphenyl

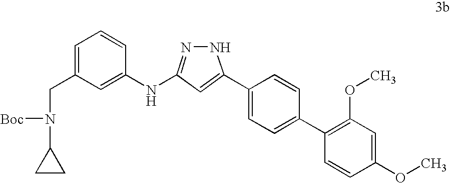

Compound 3b was synthesized using the same procedure as that of compound 1b. ¹H NMR (CDCl₃) δ 7.63 (d, 2H), 7.59 (d, 2H), 7.27 (s, 1H), 7.20 (t, 1H), 7.07 (b, 2H)), 6.76 (d, 1H), 6.57 (d, 2H), 6.34 (s, 1H), 4.39 (s, 2H), 3.85 (s, 3H), 3.81 (s, 3H), 2.50 (b, 1H), 1.43 (s, 9H), 0.71 (m, 4H). MS (ESI) M⁺+1, 541.
C. Preparation of

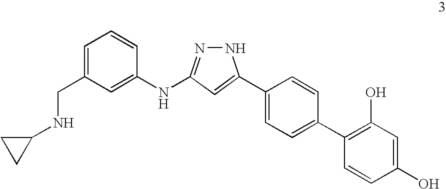

To a solution of dimethoxy biphenyl 3b (247 mg, 0.457 mmol) in 10 mL of DCM at 0° C. was added BBr₃ (1.8 ml, 1.83 mmol, 4.0 equivalents, 1M solution in DCM). The brown suspension was stirred at 0° C. and was slowly warmed to room temperature overnight. Ice cold water was then added, and the aqueous layer pH was adjusted with Na₂CO₃ to about pH 6. The desired product remained in aqueous layer as judged by its LC/MS. The aqueous layer was separated and dried under vacuum. The residue was dissolved in 2 mL of methanol. The product was purified by Dionex to give 109 mg (58%) white solids as desired acetate salt 3. ¹H NMR (CD₃OD) δ 7.67 (d, 2H), 7.60 (d, 2H), 7.38 (s, 1H), 7.26 (t, 1H), 7.16 (s, 1H), 7.13 (t, 1H), 6.85 (d, 1H), 6.39 (s, 1H), 6.36 (d, 1H), 6.30 (s, 1H), 4.08 (s, 2H), 2.60 (b, 1H), 1.94 (s, 3H), 0.73 (m, 4H). MS (ESI) M⁺+1, 413.

### Example 4

### Synthesis of Compound 4

A. Preparation of Intermediate 4a: N-Boc-N-cyclopropyl-{3-[3-(2′,4′-dimethoxy-biphenyl-4-yl)-3-oxo-propionylamino]-benzyl}-carbamic acid tert-butyl ester

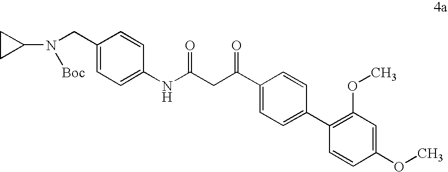

Compound 4a was synthesized using the same procedure as that of compound 1a. ¹H NMR (CDCl₃) δ 9.40 (b, 1H), 8.05 (d, 2H), 7.66 (d, 2H), 7.50 (d, 2H), 7.29 (d, 1H), 7.24 (d, 2H), 6.58 (m, 2H), 4.39 (s, 2H), 4 13 (s, 2H), 3.87 (s, 3H), 3.82 (s, 3H), 2.44 (b, 1H), 1.46 (s, 9H), 0.70 (m, 2H), 0.64 (m, 2H). MS (ESI) M⁺+1, 545.
B. Preparation of Intermediate 4b: 4′-[5-(N-Boc-4-Cyclopropylaminomethyl-phenylamino)-2H-pyrazol-3-yl]-biphenyl-2,4-dimethoxy

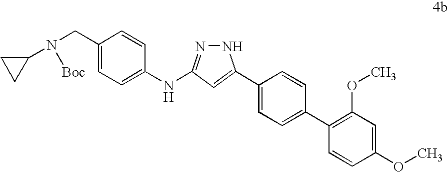

Compound 4b was synthesized using the same procedure as that of compound 1a. ¹H NMR (acetone-d₆) δ 7.75 (d, 2H), 7.69 (b, 1H), 7.55 (d, 2H), 7.39 (d, 2H), 7.27 (d, 1H), 7.16 (d, 2H), 6.66 (d, 1H), 6.61 (dd, 1H), 6.32 (s, 1H), 4.34 (s, 2H), 3.83 (s, 2H), 3.81 (s, 3H), 2.37 (b, 1H), 1.45 (s, 9H), 0.67 (m, 2H), 0.62 (m, 2H). MS (ESI) M⁺+1, 541.
C. Preparation of

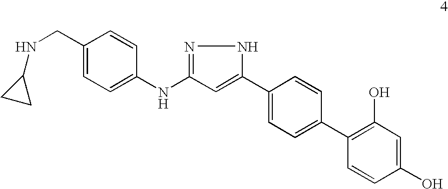

Compound 4 was synthesized using the procedures of Figures I and II (see also general preparation of Compound 3). ¹H NMR (CD₃OD) δ 7.67 (d, 2H), 7.59 (d, 2H), 7.28 (d, 4H), 7.10 (d, 1H), 6.39 (d, 1H), 6.37 (d, 1H), 6.28 (s, 1H), 4.09 (s, 2H), 2.63 (b, 1H), 1.93 (s, 3H), 0.81 (m, 4H). MS (ESI) M⁺+1, 413, 356.

### Example 5

### Preparation of Compound 5

A. Preparation of Intermediate 5a. N-Boc-N-isopropyl-{3-[3-(2′,4′-dimethoxy-biphenyl-4-yl)-3-oxo-propionylamino]-benzyl}-carbamic acid tert-butyl ester

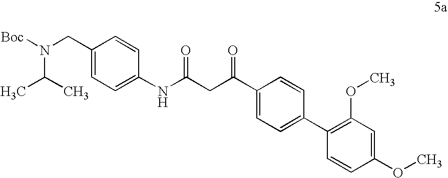

Compound 5a was synthesized using the same procedure as that of compound 1a. ¹H NMR (CDCl₃) δ 9.36 (b, 1H), 8.05 (d, 2H), 7.66 (d, 2H), 7.53 (d, 2H), 7.29 (d, 1H), 7.24 (d, 2H), 6.57 (m, 2H), 4 13 (s, 2H), 3.87 (s, 3H), 3.82 (s, 3H), 3.58 (b, 1H), 1.47 (s, 9H), 1.09 (d, 6H). MS (ESI) M⁺+1, 547.
B. Preparation of Intermediate 5b: 4′-[5-(N-Boc-4-isopropylaminomethyl-phenylamino)-2H-pyrazol-3-yl]-2,4-dimethoxybiphenyl

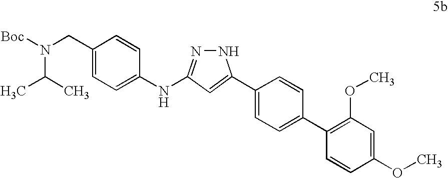

Compound 5b was synthesized using the same procedure as that of compound 1b. ¹H NMR (acetone-d₆) δ 7.74 (d, 2H), 7.66 (b, 1H), 7.55 (d, 2H), 7.38 (d, 2H), 7.27 (d, 1H), 7.16 (d, 2H), 6.66 (d, 1H), 6.62 (dd, 1H), 6.31 (s, 1H), 4.31 (s, 2H), 3.84 (s, 3H), 3.82 (s, 3H), 2.85 (b, 1H), 1.43 (s, 9H), 1.1 (d, 6H). MS (ESI) M⁺+1, 543.
C. Preparation of

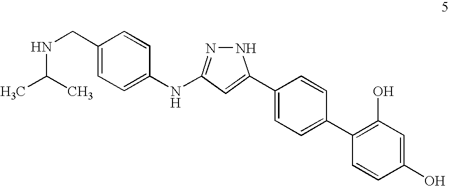

Compound 5 was synthesized according to Figures I and II (see also preparation of Compound 3). ¹H NMR (CD₃OD) δ 7.66 (d, 2H), 7.60 (d, 2H), 7.31 (dd, 4H), 7.12 (d, 1H), 6.38 (m, 2H), 6.28 (s, 1H), 4.08 (s, 2H), 3.39 (q, 1H), 1.92 (s, 3H), 1.38 (d, 6H). MS (ESI) M⁺+1, 356.

### Example 6

### Preparation of Compound 6

A. Preparation of Intermediate 6a. N-Boc-isopropyl-{3-[3-(2′,4′-dimethoxy-5′-methyl biphenyl-4-yl)-3-oxo-propionylamino]-benzyl}-carbamic acid tert-butyl ester

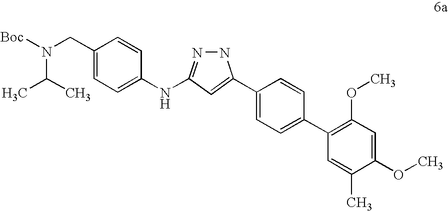

Compound 6a was synthesized using the same procedure as that of compound 1b. ¹H NMR (CDCl₃) δ 9.39 (b, 1H), 8.03 (d, 2H), 7.66 (d, 2H), 7.52 (d, 2H), 7.20 (d, 2H), 7.11 (s, 1H), 6.52 (s, 1H), 4.30 (b, 2H), 4 12 (s, 2H), 3.89 (s, 3H), 3.82 (s, 3H), 2.19 (s, 3H), 1.38 (b, 9H), 1.09 (d, 6H).
B. Preparation of Intermediate 6b. 4′-[5-(N-Boc-4-Cyclopropylaminomethyl-phenylamino)-2H-pyrazol-3-yl]-2,4-dimethoxy-5-methylbiphenyl

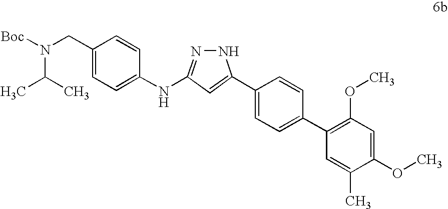

Compound 6b was synthesized using the same procedure as that of compound 1b. ¹H NMR (methanol-d₄) δ 7.65 (d, 2H), 7.51 (d, 2H), 7.15 (d, 1H), 7.11 (d, 2H), 6.65 (s, 1H), 6.27 (s, 1H), 4.31 (s, 2H), 3.88 (s, 3H), 3.80 (s, 3H), 2.15 (s, 3H), 1.43 (b, 9H), 1.12 (d, 6H).
C. Preparation of

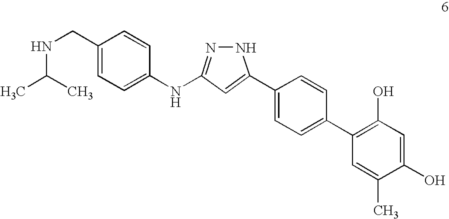

Compound 6 was synthesized according to Figures I and II. ¹H NMR (methanol-d₄) δ 7.65 (d, 2H), 7.60 (d, 2H), 7.29 (s, 4H), 6.99 (s, 1H), 6.40 (s, 1H), 6.28 (s, 1H), 4.04 (s, 2H), 3.35 (m, 1H), 2.13 (s, 3H), 1.35 (d, 6H).

### Example 7

### Preparation of Compound 7

A. Preparation of Intermediate 7a. N-Boc-isopropyl-{3-[3-(2′,440 -dimethoxy-6′-methylbiphenyl-4-yl)-3-oxo-propionylamino]-benzyl}-carbamic acid tert-butyl ester

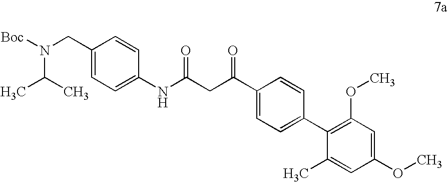

Compound 7a was synthesized using the same procedure as that of compound 1a. ¹H NMR (CDCl₃) δ 9.35 (b, 1H), 8.06 (d, 2H), 7.53 (d, 2H), 7.36 (d, 2H), 7.20 (d, 2H), 6.44 (d, 1H), 6.40 (d, 1H), 4.30 (b, 2H), 4 12 (s, 2H), 3.84 (s, 3H), 3.67 (s, 3H), 2.06 (s, 3H), 1.38 (s, 9H), 1.08 (d, 6H).
B. Preparation of Intermediate 7b. 4′-[5-(N-Boc-4-Cyclopropylaminomethyl-phenylamino)-2H-pyrazol-3-yl]-2,4-dimethoxy-6-methylbiphenyl

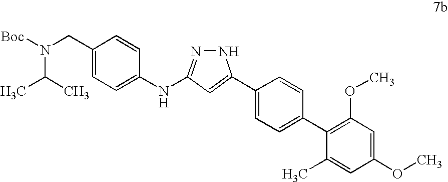

Compound 7b was synthesized using the same procedure as that of compound 1b. ¹H NMR (methanol-d₄) δ 7.68 (d, 2H), 7.19 (d, 2H), 7.14 (d, 1H), 7.12 (d, 2H), 6.44 (m, 2H), 6.29 (s, 1H), 4.32 (s, 2H), 3.81 (s, 3H), 3.66 (s, 3H), 2.05 (s, 3H), 1.43 (b, 9H), 1.12 (d, 6H).
C. Preparation of

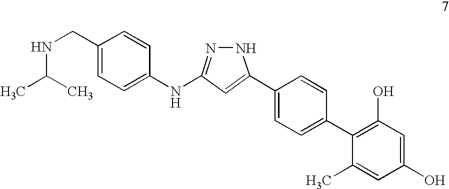

Compound 7 was synthesized according to Figures I and II. ¹H NMR (methanol-d₄) 7.69 (d, 2H), 7.30 (s, 4H), 7.37 (d, 1H), 6.30 (s, 1H), 6.24 (m, 2H), 4.05 (s, 2H), 3.35 (m, 1H), 2.00 (s, 3H), 1.35 (d, 6H).

### Example 8

### Preparation of Compound 8

A. Preparation of Intermediate 8a. Cyclopropyl-{3-[3-(6′-chloro-2′,4′-dimethoxy-biphenyl-4-yl)-3-oxo-propionylamino]-benzyl}-carbamic acid tert-butyl ester

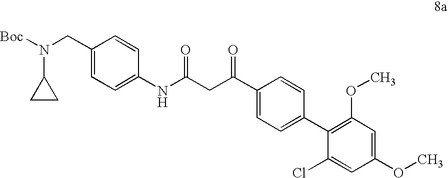

Compound 8a was synthesized using the same procedure as that of compound 1a. ¹H NMR (CDCl₃) δ 9.36 (b, 1H), 8.10 (d, 2H), 7.55 (d, 2H), 7.45 (d, 2H), 7.23 (d, 1H), 6.65 (d, 1H), 6.46 (d, 1H), 4.39 (s, 2H), 4 15 (s, 2H), 3.85 (s, 3H), 3.71 (s, 3H), 2.42 (b, 1H), 1.46 (s, 9H), 0.70 (m, 2H), 0.63 (m, 2H). MS (ESI) M⁺+1, 580.
B. Preparation of Intermediate 8b. 4′-[5-(N-Boc-4-Cyclopropylaminomethyl-phenylamino)-2H-pyrazol-3-yl]-biphenyl-5-chloro-2,4-dimethoxy

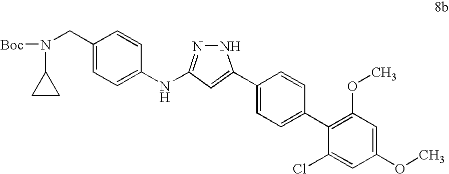

Compound 8b was synthesized using the same procedure as that of compound 1b. ¹H NMR (CDCl₃) δ 7.62 (d, 2H), 7.35 (d, 2H), 7.17 (d, 2H), 7.09 (d, 2H), 6.65 (d, 1H), 6.47 (d, 1H), 6.33 (s, 1H), 4.36 (s, 2H), 3.85 (s, 3H), 3.71 (s, 3H), 2.43 (b, 1H), 1.47 (s, 9H), 0.70 (m, 2H), 0.64 (m, 2H). MS (ESI) M⁺+1, 576.
C. Preparation of

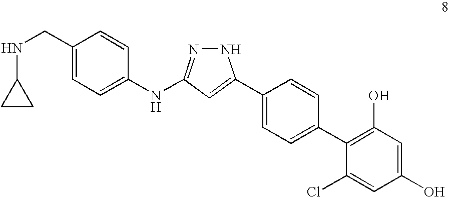

Compound 8 was synthesized using the procedures of Figures I and II. ¹H NMR (CD₃OD) δ 7.68 (d, 2H), 7.31 (d, 2H), 7.24 (b, 4H), 6.44 (d, 1H), 6.32 (d, 1H), 6.30 (s, 1H), 3.95 (s, 2H), 2.44 (b, 1H), 1.91 (s, 3H), 0.69 (m, 4H). MS (ESI) M⁺+1, 390.

### Example 9

### Preparation of Compound 9

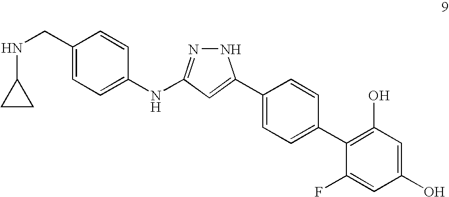

Compound 9 was synthesized according to Figures I and II. ¹H NMR (CD₃OD) δ 7.67 (d, 2H), 7.45 (d, 2H), 7.30 (s, 4H), 6.30 (s, 1H), 6.22 (s, 1H), 6.13 (q, 1H), 4.16 (s, 2H), 2.70 (m, 1H), 0.85 (m, 4H).

### Example 10

### Preparation of Compound 10

A. Preparation of Intermediate 10a. Cyclopropylmethyl-{3-[3-(2′,4′-dimethoxy-biphenyl-4-yl)-3-oxo-propionylamino]-benzyl}-carbamic acid tert-butyl ester

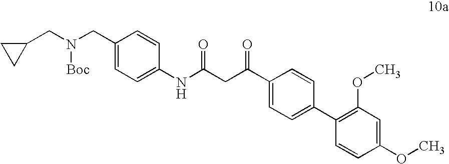

Compound 10a was synthesized using the same procedure as that of compound 1a. ¹H NMR (CDCl₃) δ 9.40 (b, 1H), 8.05 (d, 2H), 7.66 (d, 2H), 7.50 (d, 2H), 7.29 (d, 1H), 7.24 (d, 2H), 6.58 (m, 2H), 4.39 (s, 2H), 4 13 (s, 2H), 3.87 (s, 3H), 3.82 (s, 3H), 2.44 (b, 1H), 1.46 (s, 9H), 0.70 (m, 2H), 0.64 (m, 2H). MS (ESI) M⁺+1, 559.
B. Preparation of Intermediate 10b. 4′-[5-(4-Cyclopropylmethylaminomethyl-phenylamino)-2H-pyrazol-3-yl]-biphenyl-2,4-dimethoxy

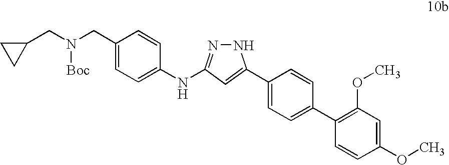

Compound 10b was synthesized using the same procedure as that of compound 1b. ¹H NMR (CDCl₃) δ 7.56 (d, 2H), 7.25 (d, 1H), 7.11 (b, 2H), 6.56 (m, 2H), 6.29 (s, 1H), 4.47 (s, 2H), 3.85 (s, 3H), 3.80 (s, 3H), 3.02 (b, 2H), 1.47 (s, 9H), 0.94 (m, 1H), 0.43 (m, 2H), 0.14 (m, 2H). LC/MS (ESI) 555
C. Preparation of

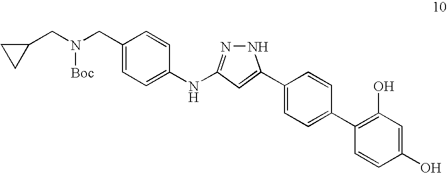

Compound 10 was synthesized using the procedures of Figures I and II. ¹H NMR (CD₃OD) δ 7.66 (d, 2H), 7.60 (d, 2H), 7.29 (b, 4H), 7.12 (d, 1H), 6.40 (m, 2H), 6.29 (s, 1H), 4.06 (s, 2H), 2.85 (d, 2H), 1.89 (s, 2H), 1.08 (m, 1H), 0.69 (m, 2H), 0.36 (m, 2H). LC/MS (ESI) 356

### Example 11

### Preparation of Compound 11

A. Preparation of Intermediate 11a. 3-(2′,4′-Dimethoxy-1,1′-biphenyl-4-yl)-3-oxo-N-pyrimidin-2-ylpropanamide

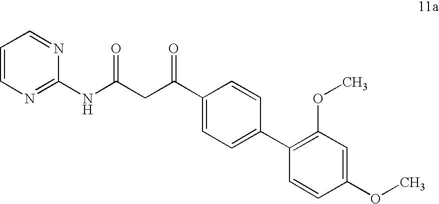

Compound 11a was synthesized using the same procedure as that of compound 1a. ¹H NMR (CDCl₃) δ: 3.74 (3H, s), 3.78 (5H, s), 3.83 (5H, s), 6.54 (3H, m), 7.23 (2H, d, J=8.34 Hz), 7.59 (3H, m), 7.94 (2H, m). MS (APCI positive)
B. Preparation of Intermediate 11b. N-[3-(2′,4′-Dimethoxy-1,1′-biphenyl-4-yl)-1H-pyrazol-5-yl]pyrimidin-2-amine

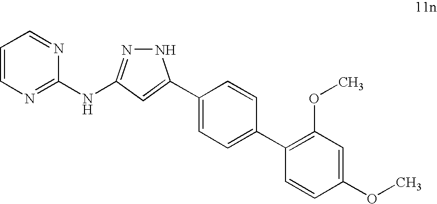

Compound 11b was synthesized using the same procedure as that of compound 1b. ¹H NMR (CDCl₃) δ: 3.78 (3H, s), 3.84 (3H, s), 6.55 (4H, m), 7.05 (1H, bs), 7.23 (1H, d, J=8.84 Hz), 7.55 (2H, d, J=8.08 Hz), 7.62 (2H, d, J=7.83 Hz), 8.30 (1H, bs), 8.72 (1H, s).
C. Preparation of

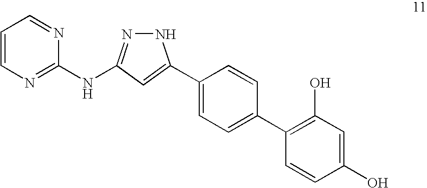

Compound 11 was synthesized using the procedures of Figures I and II. ¹H NMR (MeOD-d₃) δ: 6.39 (2H, m), 6.77 (1H, s), 7.14 (2H, d, J=8.10 Hz), 7.62 (2H, d J=8.48 Hz), 7.70 (2H, d, J=8.46 Hz), 8.23 (1H, d, J=5.84 Hz), 8.59 (1H, s). MS (APCI positive) 346.1.

### Example 12

### Preparation of Compound 12

A. Preparation of Intermediate 12a. Ethyl 5-{[3-(2′,4′-dimethoxy-1,1′-biphenyl-4-yl)-3-oxopropanoyl]amino}pyridine-2-carboxylate

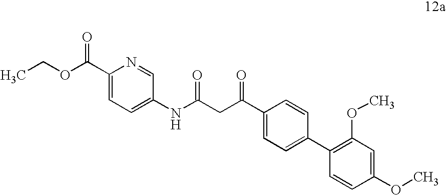

Compound 12a was synthesized using the same procedure as that of compound 1a. ¹H NMR (DMSO-d₆) δ 10.80 (s, 1H), 8.79 (s, 1H), 8.27 (d, 1H), 8.03 (m, 3H), 7.61 (d, 2H), 7.29 (d, 1H), 6.66 (t, 2H), 4.32 (q, 2H), 4.25 (s, 2H), 3.81 (s, 3H), 3.78 (s, 3H), 1.31 (t, 3H).
B. Preparation of Intermediate 12b. 5-[5-(2′,4′-Dimethoxy-biphenyl-4-yl)-2H-Pyrazol-3-ylamino]-pyridine-2-carboxylic acid ethyl ester

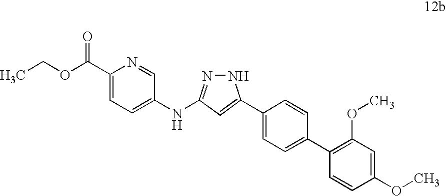

Compound 12b was synthesized using the same procedure as that of compound 1b. ¹H NMR (DMSO-d₆) δ 9.52 (b, 1H), 8.64 (s, 1H), 7.96 (m, 2H), 7.74 (d, 1H), 7.51 (d, 2H), 7.27 (d, 1H), 6.67 (s, 1H), 6.63 (dd, 1H), 6.36 (s, 1H), 4.28 (q, 2H), 3.80 (s, 3H), 3.77 (s, 3H), 1.30 (t, 2H).
C. Preparation of Intermediate 12c. 5-{[3-(2′,4′-dihydroxy-1,1′-biphenyl-4-yl)-1H-pyrazol-5-yl]amino}pyridine-2-carboxylic acid

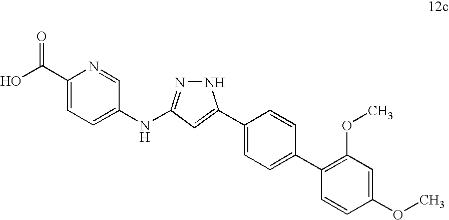

Compound 12c was synthesized using the procedure of Figure II, starting with 5-[5-(2′,4′-dimethoxy-biphenyl-4-yl)-2H-Pyrazol-3-ylamino]-pyridine-2-carboxylic acid ethyl ester. The ethyl ester was hydrolyzed by BBr₃ as well. ¹H NMR (CD₃OD) δ 9.22 (s, 1H), 8.41 (s, 2H), 7.73 (q, 5H), 7.22 (d, 1H), 6.48 (t, 3H).
D. Preparation of

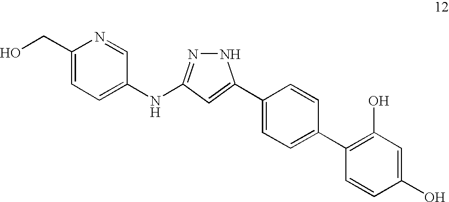

To a solution of 5-[5-(2′,4′-Dimethoxy-biphenyl-4-yl)-2H-Pyrazol-3-ylamino]-pyridine-2-carboxylic acid ethyl ester (26 mg, 0.0586 mmol) in 1.2 mL of dry THF at room temperature was added LAH (0.6 mL, 1.0 M in diethyl ether). The reduction finished within 1.5 hour as monitored by LC/MS. Ice water was added and the reaction was extracted with ethyl acetate. The organic layer was separated and concentrated to an oil. The product was purified by column chromatography with pure ethyl acetate as solvent to give 9.7 mg of 4′-[5-(6-Hydroxymethyl-pyridin-3-ylamino)-2H-pyrazol-3-yl]-biphenyl-2,4-dimethoxy. This compound was demethylated using BBr₃ using the same procedure as that of AG-121011. ¹H NMR (acetone-d₆) δ 8.99 (b, 1H), 8.58 (s, 1H), 8.53 (s, 1H), 8.18 (d, 1H), 7.78 (d, 2H), 7.64 (d, 2H), 7.42 (b, 1H), 7.17 (d, 1H), 6.63 (d, 1H), 6.46 (dd, 1H), 6.41 (s, 1H), 4.85 (s, 2H).

### Example 13

### Preparation of Compound 13

A. Preparation of Intermediate 13a. 4-[3-(2′,4′-Dimethoxy-biphenyl-4-yl)-3-oxo-propionylamino]-pyridine-2-carboxylic acid ethyl ester

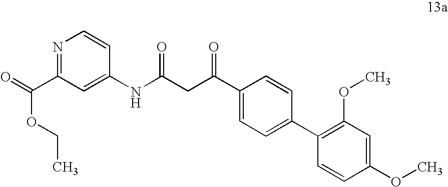

Compound 13a was synthesized using a procedure analogous to that of compound 1a. ¹H NMR (DMSO-d₆) δ 10.85 (s, 1H), 8.56 (dd, 1H), 8.25 (d, 1H), 7.99 (d, 2H), 7.77 (m, 1H), 7.63 (d, 2H), 7.30 (d, 1H), 6.69 (d, 1H), 6.65 (dd, 1H), 4.32 (q, 2H), 4.23 (s, 2H), 3.81 (s, 3H), 3.78 (s, 3H), 1.31 (s, 3H).
B. Preparation of

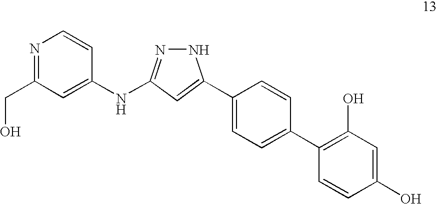

Compound 13 was synthesized using a procedure analogous to that of Compound 12. ¹H NMR (CD₃OD) δ 8.12 (d, 1H), 7.68 (d, 2H), 7.62 (d, 2H), 7.47 (s, 1H), 7.41 (m, 1H), 7.32 (d, 1H), 6.43 (s, 1H), 6.39 (m, 2H), 4.69 (s, 2H), 1.95 (s, 3H).

### Example 14

### Preparation of Compound 14

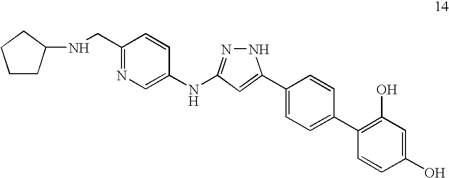

Example 14 was synthesized using a procedure analogous to that used for Compound 13. ¹H NMR (CD₃OD) δ 8.57 (d, 1H), 7.85 (q, 1H), 7.65 (d, 2H), 7.61 (d, 2H), 7.33 (d, 1H), 7.12 (d, 1H), 6.39 (q, 2H), 6.27 (s, 1H), 4.19 (s, 2H), 2.64 (m, 1H), 2.15 (m, 2H), 1.83 (m, 2H), 1.68 (m, 4H).

### Example 15

### Preparation of Compound 15

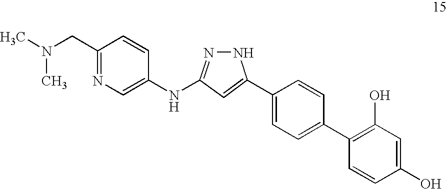

Compound 15 was synthesized using a procedure analogous to that used for Compound 14. ¹H NMR (DMSO-d₆) δ 12.51 (s, 1H), 9.47 (s, 1H), 9.38 (s, 1H), 8.78 (s, 1H), 8.55 (s, 1H), 7.86 (s, 1H), 7.69 (d, 2H), 7.55 (d, 2H), 7.27 (d, 1H), 7.10 (d, 1H), 6.42 (d, 1H), 6.31 (q, 1H), 6.25 (s, 1H), 3.30 (s, 2H), 2.32 (s, 6H).

### Example 16

### Preparation of Compound 16

A. Preparation of Intermediate 16a. Ethyl 5-{[3-(2′,4′-dimethoxy-1,1′-biphenyl-4-yl)-3-oxopropanoyl]amino}pyridine-2-methylamide

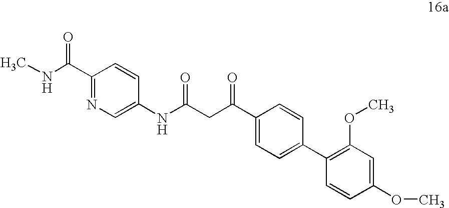

Compound 16a was synthesized using the same procedure as that of compound 1a. ¹H NMR (DMSO-d₆) δ 10.71 (b, 1H), 8.80 (d, 1H), 8.66 (d, 1H), 8.17 (dd, 2H), 7.63 (d, 2H), 7.30 (d, 1H), 6.69 (d, 1H), 6.65 (m, 1H), 4.23 (s, 2H), 3.81 (s, 3H), 3.78 (s, 3H), 2.79 (d, 3H).

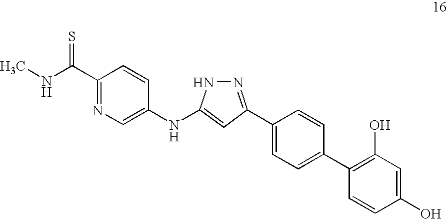

Compound 16 was synthesized according to compound 14. ¹H NMR (DMSO-d₆) δ 9.06 (d, 1H), 9.02 (b, 1H), 8.97 (d, 1H), 8.53 (dd, 1H), 8.20 (d, 2H), 8.10 (d, 2H), 7.63 (d, 1H), 6.99 (d, 1H), 6.92 (d, 1H), 6.83 (s, 1H), 3.76 (d, 3H). LC/MS (APCI) 418.

### Preparation of Certain Intermediates According to Figure III is Described in Examples 17 to 20

### Example 17

### Preparation of Compound 17

A. Preparation of Intermediate 17a. N-pyridin-2-yl acetamide

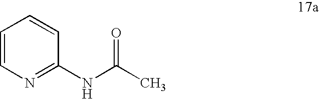

To a solution of pyridin-2-amine (4.14 g, 44.0 mmol) in anhydrous dimethylformamide (25 mL), cooled to 0° C., was added anhydrous pyridine (4.4 mL) followed by slow addition of acetic anhydride (4.6 mL, 48.4 mmol). After addition was complete, the reaction was allowed to warm to room temperature and stir overnight. After an aqueous sodium bicarbonate workup with ethyl acetate extraction, the organic layers were pooled and concentrated to a yellow oil which quickly crystallized to give the title compound (6.15 g, quant.) ¹H NMR (CDCl₃) δ: 2.19 (3H, s), 7.03 (1H, m), 7.60 (1H, t, J=8.84 Hz), 8.18 (1H, d, J=8.33 Hz), 8.26 (1H, d, J=3.79 Hz), 8.50 (1H, bs).
B. Preparation of Intermediate 17b. N-pyridin-2-ylethanethioamide

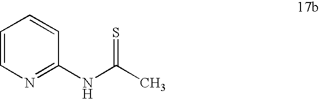

To a toluene solution of N-pyridin-2-ylacetamide (17a) (1.88 g, 13.8 mmol) was added Lawesson's Reagent (6.72 g, 16.6 mmol). The suspension was heated to reflux for 1 hour then concentrated to a yellow oil and purified by flash column to yield the title compound as a yellow oil (627 mg, 33%). ¹H NMR (CDCl₃) δ: 2.78 (3H, s), 7.21 (1H, bs), 7.81 (1H, t, J=7.83), 8.42 (1H, bs), 9.14 (1H, bs). MS (APCI positive) 153.0.
C. Preparation of Intermediate 17c. 3-(2′,4′-dimethoxy-1,1′-biphenyl-4-yl)-3-oxo-N-pyridin-2-ylpropanethioamide

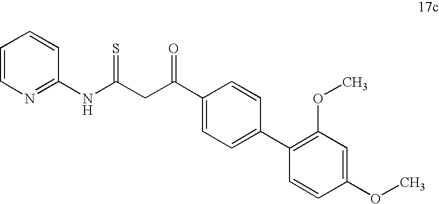

To a solution of N-pyridin-2-ylethanethioamide (17b) (325 mg, 2.1 mmol) in anhydrous tetrahydrofuran (20 mL) stirring at −78° C., was added 1.7M t-BuLi in pentane (2.5 mL, 4.27 mmol) dropwise. The reaction was warmed to 0° C. for 1 hour then cooled back down to −78° C. at which point a solution of N,2′,4′-trimethoxy-N-methyl-1,1′-biphenyl-4-carboxamide (322 mg, 1.1 mmol) in anhydrous tetrahydrofuran (ca. 5 mL) was added dropwise. The reaction was then allow to warm to room temperature while stirring overnight. The reaction was quenched and the product precipitated by addition of 1 mL of 1:1 methanol:acetic acid. Filtration yielded the title compound as a yellow powder (210 mg, 49%). ¹H NMR (CDCl₃) δ: 3.81 (3H, s), 3.86 (3H, s), 4.63 (2H, s), 6.56 (2H, m), 7.16 (2H, bm), 7.29 (1H, d, J=8.34 Hz), 7.64 (2H, d, J=8.34 Hz), 7.75 (1H, m), 8.11 (2H, d, J=8.34 Hz), 8.44 (1H, m). MS (APCI positive) 393.1.
D. Preparation of Intermediate 17d. N-[5-(2′,4′-dimethoxy-1,1′-biphenyl-4-yl)-1H-pyrazol-3-yl]pyridin-2-amine

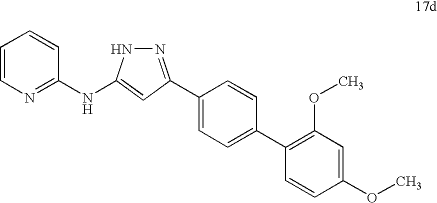

Intermediate 17d was synthesized using procedures analogous to those used in the synthesis of compound 2. ¹H NMR (CDCl₃) δ: 3.81 (3H, s), 3.85 (3H, s), 6.29 (1H, s), 6.56 (2H, m), 6.80 (1H, m), 7.00 (1H, d, J=8.33 Hz), 7.10 (1H, bs), 7.28 (1H, dd, J=7.58 Hz), 7.57 (3H, m), 7.71 (2H, d, J=8.33 Hz), 8.22 (1H, d, J=5.05 Hz). MS (APCI positive) 373.1.
E. Preparation of

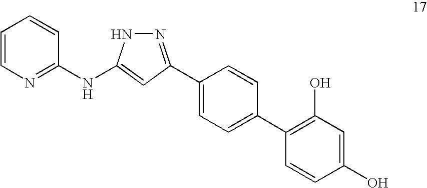

Compound 17 was synthesized from compound 17d using a procedure according to Figure II (See also Example 3C). ¹H NMR (DMSO-d₆) δ: 6.30 (1H, dd, J=8.33 Hz), 6.41 (1H, s), 6.74 (2H, bm), 7.11 (1H, d, J=8.33 Hz), 7.25 (1H, bs), 7.56 (3H, m), 7.66 (2H, m), 8.14 (1H, s), 9.36 (1H, bm), 12.54 (1H, s). MS (APCI positive) 345.1.

### Example 18

### Preparation of Compound 18

A. Preparation of Intermediate 18a. N-pyridin-3-ylacetamide

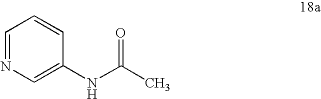

Intermediate 18a was synthesized using a procedure analogous to that of compound 17a. ¹H NMR (DMSO-d₆) δ: 2.05 (3H, s), 7.31 (1H, m), 7.99 (1H, m), 8.21 (1H, m), 8.69 (1H, s), 10.13 (1H, s).
B. Preparation of Intermediate 18b. N-pyridin-3-ylethanethioamide

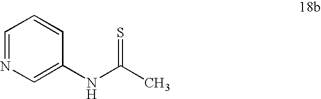

Intermediate 18b was synthesized using a procedure analogous to that of compound 17b. ¹H NMR (CDCl₃) δ: 2.76 (3H, s), 7.37 (1H, m), 8.46 (2H, m), 8.62 (1H, s), 9.26 (1H, bs). MS (APCI positive) 153.0.
C. Preparation of Intermediate 18c. 3-(2′,4′-dimethoxy-1,1′-biphenyl-4-yl)-3-oxo-N-pyridin-3-ylpropanethioamide

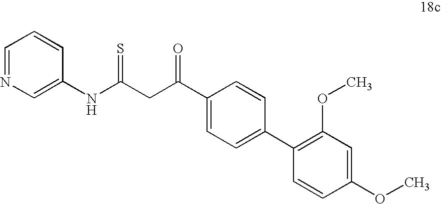

Intermediate 18c was synthesized using a procedure analogous to that of compound 17c. MS (APCI positive) 393.1.
D. Preparation of Intermediate 18d. N-[5-(2′,4′-dimethoxy-1,1′-biphenyl-4-yl)-1H-pyrazol-3-yl]pyridin-3-amine

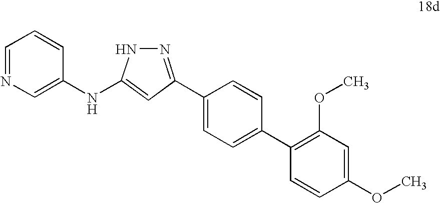

Compound 18d was prepared using procedures analogous to those as previously described for compound 1b. ¹H NMR (MeOD-d₃) δ: 3.71 (3H, s), 3.74 (3H, s), 6.19 (1H, s), 6.54 (2H, m), 7.17 (2H, m), 7.45 (2H, d, J=8.34 Hz), 7.59 (2H, d, J=8.09 Hz), 7.74 (1H, m), 7.84 (1H, m), 8.40 (1H, bs). MS (APCI positive) 373.1.
E. Preparation of

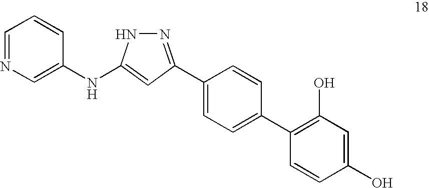

Compound 18 was prepared according a procedure analogous to that presented for Compound 17.

¹H NMR (DMSO-d₆) δ: 6.31 (2H, m), 6.42 (1H, s), 7.12 (1H, d, J=8.08 Hz), 7.58 (3H, m), 7.72 (2H, d, J=8.34 Hz), 8.11 (2H, m), 8.83 (1H, bs), 9.32 (1H, bs), 9.38 (1H, s), 9.47 (1H, s), 12.69 (1H, bs). MS (APCI positive) 345.1.

### Example 19

### Preparation of Compound 19

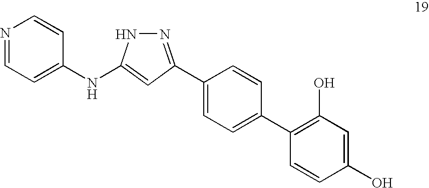

Compound 19 was prepared according to procedures analogous to those for Compound 17.

¹H NMR (CD₃OD) δ 8.19 (d, 2H), 7.68 (d, 2H), 7.62 (d, 2H), 7.41 (d, 2H), 7.13 (d, 1H), 6.39 (t, 3H).

### Example 20

### Preparation of Compound 20

A. Preparation of Intermediate 20a. N-(5-thiazole)-thioacetamide

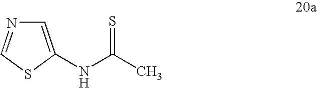

Intermediate 20a was synthesized using a procedure analogous to that of compound 17a. ¹H NMR (CDCl₃) δ: 12.40 (b, 1H), 7.43 (s, 1H), 6.99 (s, 1H), 2.34 (3H, s).
B. Preparation of Intermediate 20b. N-[3-(2′,4′-dimethoxy-1,1′-biphenyl-4-yl)-1H-pyrazol-5-yl]-1,3-thiazol-5-amine

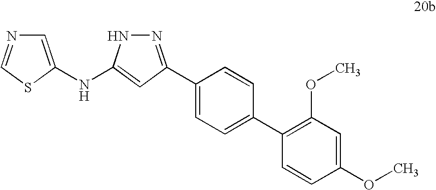

Intermediate 20b was prepared by analogy to compound 19d. ¹H NMR (DMSO-d₆) δ 12.76 (s, 1H), 10.62 (s, 1H), 7.70 (d, 2H), 7.51 (d, 2H), 7.25 (t, 2H), 6.88 (d, 1H), 6.64 (t, 2H), 6.35 (s, 1H), 3.80 (s, 3H), 3.77 (s, 3H).
C. Preparation of

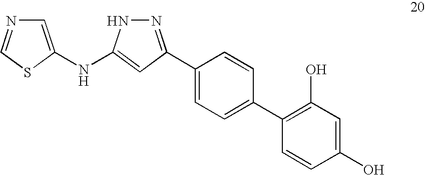

Compound 20 was Prepared According to Compound 17

¹H NMR (DMSO-d₆) δ 11.14 (b, 1H), 9.45 (b, 2H), 7.68 (d, 2H), 7.57 (d, 2H), 7.33 (d, 1H), 7.11 (d, 1H), 6.98 (d, 1H), 6.41 (t, 2H), 6.32 (q, 1H).

### Compound 21 was Synthesized According to Figure IV

### Example 21

### Preparation of Compound 21

A. Preparation of Intermediate 21a. 5-(4-Bromophenyl)-N-phenyl-1H-pyrazol-3-amine

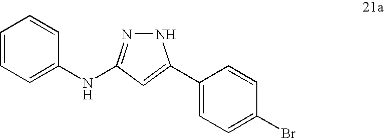

To a suspension of 4-phenyl-3-thiosemicarbazide (1.00 g, 6.0 mmol) in absolute ethanol (30 mL) was added 2,4′-dibromoacetophenone (1.67 g, 6.0 mmol). After stirring for 2.5 hours, saturated ethanolic HCl (20 mL) was added and the reaction was heated to reflux overnight. An additional 30 mL ethanolic HCl was added and reflux was continued for another 9 hours, after which the reaction was stirred at room temperature overnight. The pH was adjusted to 8 with 0.5M NaOH and an aqueous workup performed, extracting with ethyl acetate. Product was purified by column chromatography using ethyl acetate:hexanes 1:10 then 1:5 and finally 1:2. The title compound was isolated as a pale yellow solid (533 mg, 28%). ¹H NMR (CD₃OD) δ: 6.19 (1H, s), 6.68 (1H, bs), 7.10 (4H, bm), 7.47 (4H, bm). MS (APCI positive) 315.0.
B. Preparation of Intermediate 21b. 5-(2′,4′-dimethoxy-1,1′-biphenyl-4-yl)-N-phenyl-1H-pyrazol-3-amine

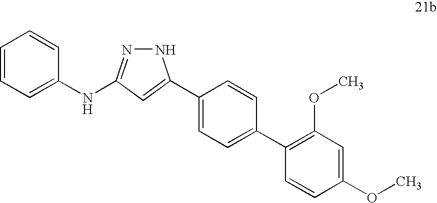

To a solution of the bromide (above) in dioxane (10 mL) was added 2,4-dimethoxyboronic acid (371 mg, 2.0 mmol), potassium carbonate (352 mg, 2.5 mmol), triethylamine (0.4 mL, 2.5 mmol) and water (0.3 mL). The system was vacuum flushed with nitrogen, then dichloro-di-triphenylphosphoro palladium catalyst (60 mg, 5 mol%) was added. Reaction heated at 90° C. overnight. Purified by column using ethyl acetate:hexanes 1:2 isolating the title compound as an amber oil (46 mg, 7%). ¹H NMR (CD₃OD) δ: 3.70 (3H, s), 3.74 (3H, s), 6.20 (1H, s), 6.54 (2H, bm), 6.69 (1H, bs), 7.11 (6H, bm), 7.44 (2H, d, J=7.83 Hz), 7.57 (2H, m). MS (APCI positive) 372.2.
C. Preparation of

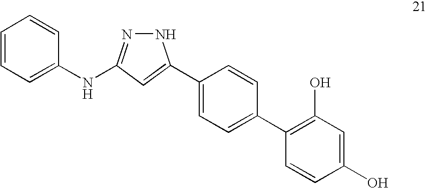

Compound 21 was synthesized according to the methods of Figure II. ¹H NMR (CDCl₃) δ: 6.19 (1H, s), 6.30 (2H, m), 6.68 (1H, m), 7.02 (1H, d, J=7.83 Hz), 7.09 (4H, m), 7.51 (2H, d, J=8.59 Hz), 7.56 (2H, d, J=8.34 Hz).

### Example 22

### Preparation of Compound 22

Compound 22 was prepared according to methods of Figure II condensing Intermediate D with the corresponding amine prepared according to Figure V:
A. Preparation of Intermediate 22a. N-(sec-butyl)-(4-nitrobenzyl)amine

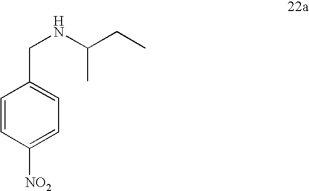

To a solution of sec-butylamine (2.6 mL, 90 mmol) in anhydrous DMF (15 mL) was added a solution of 4-nitro-benzylbromide (1.99 g, 9.2 mmol) in anhydrous DMF (10 mL) dropwise via addition funnel while stirring overnight at room temperature. The product was purified by flash column chromatography using ethyl acetate:hexane 1:5, 1:2, 1:1 and methanol:chloroform 1:20 to afford the title compound in quantitative yield.

¹H NMR (400 MHz, CDCl₃) δ ppm 0.90 (t, J=7.33 Hz, 3 H) 1.06 (d, J=6.06 Hz, 3 H) 1.44 (m, 2 H) 2.59 (m, 1H) 3.88 (m, 2 H) 7.50 (d, J=8.59 Hz, 2 H) 8.16 (d, J=8.59 Hz, 2 H)
B. Preparation of Intermediate 22b. N-(sec-butyl)-N-Boc-(4-nitrobenzyl)amine

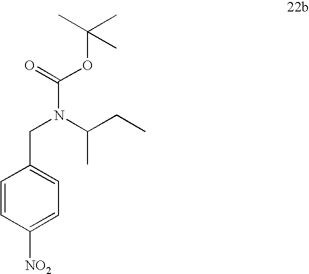

To a solution of N-(sec-butyl)-N-(4-nitrobenzyl)amine (22a) (2.2 g, 10.6 mmol) in anhydrous acetonitrile (30 mL) was added triethylamine (1.5 mL, 10.6 mmol) followed by di-t-butyldicarbonate (2.3 g, 10.6 mmol). After stirring for 3 hours at room temperature, the reaction was plug filtered through silica gel, eluting with ethyl acetate:hexane 1:1, isolating the title compound as a light yellow oil.

¹H NMR (400 MHz, CDCl₃) δ ppm 1.06 (bs, 3 H) 1.28 (bs, 5 H) 1.49 (s, 9 H) 4.30 (bm, 3 H) 7.38 (d, J=5.56 Hz, 2 H) 8.14 (d, J=8.08 Hz, 2 H)
C. Preparation of Intermediate 22c. N-(sec-butyl)-N-Boc-(4-aminobenzyl)amine

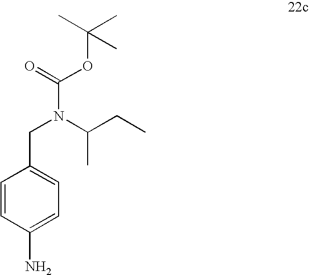

To a solution of 22b in methanol (10 mL), vacuum flushed with nitrogen, was added a small spatula tip of 10% palladium on carbon. Hydrogen was then introduced under balloon pressure. After 2 hours, although not completely converted, the nitro and aniline species were separated by flash column chromatography using ethyl acetate:hexane 1:3 and 1:2.

¹H NMR (400 MHz, CDCl₃) δ ppm 1.04 (d, 3 H) 1.38 (bs, 9 H) 1.49 (bm, 5 H) 3.58 (s, 2 H) 4.26 (s, 1 H) 6.62 (d, 2 H) 7.04 (bs, 2H).
D. Preparation of Intermediate 22d.

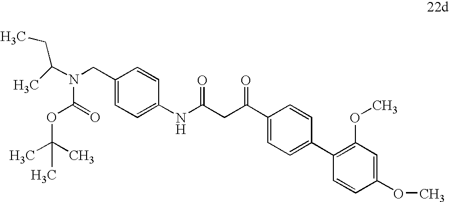

Intermediate 22d was prepared using the method described for compound 1a.

¹H NMR (400 MHz, CDCl₃) δ ppm 0.81(bs, 3H), 0.87 (t, 3H), 1.04 (bs, 3H), 1.35 (m, 9H), 1.49 (m, 5H), 3.80 (m, 1H), 3.81 (s, 3H), 3.86 (s, 3H), 4.11 (s, 2H), 4.33 (bm, 2H), 6.57 (m, 2H), 7.25 (bm, 3H), 7.53 (d, 2H), 7.66 (d, 2H), 8.05 (d, 2H).
E. Preparation of Intermediate 22e.

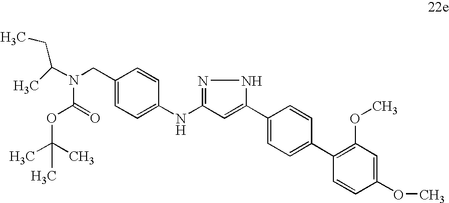

Intermediate 22e was prepared using the method described for compound 1b. ¹H NMR (400 MHz, CDCl₃) δ ppm 0.81(bs, 3H), 0.87 (t, 3H), 1.07 (d, 2H), 1.26 (m, 5H), 1.42 (mm, 9H), 1.60 (m, 1H), 3.80 (s, 3H), 3.83 (m, 1H), 3.85 (s, 3H), 6.29 (sm, 1H), 6.58 (m, 2H), 7.09 (d, 2H), 7.14 (bs, 2H), 7.25 (m, 1H), 7.57 (m, 4H)
F. Preparation of

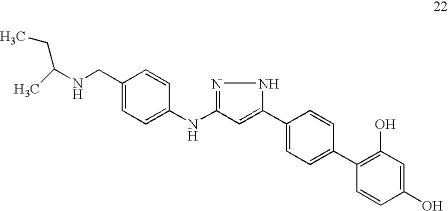

Compound 22 was synthesized from compound 22e using a procedure according to Figure I. ¹H NMR (400 MHz, Methanol-d₄) δ 0.88 (m, 3H), 1.23 (d, 3H) 1.45 (m, 1H), 1.77 (m, 1H), 3.07 (m, 1H), 3.97 (q, 2H), 6.16 (s, 1H), 6.28 (m, 2H), 7.00 (d, 1H), 7.18 (m, 4H), 7.49 (d, 2H), 7.55 (d, 2H)

### Examples 23 to 26

Compounds of Examples 23 to 26 were synthesized according to Figure VI by coupling an appropriate amino derivative with a common pyrazole biphenyl precursor functionalized with a pyridyl carbonyl precursor followed by reduction of the newly formed amide bond.

The aldehyde precursor G, useful for the preparation of the compounds of examples 23-26, is prepared from the corresponding cyano precursor (Intermediate H) which exists in equilibrium with a keto-enol tautomer: 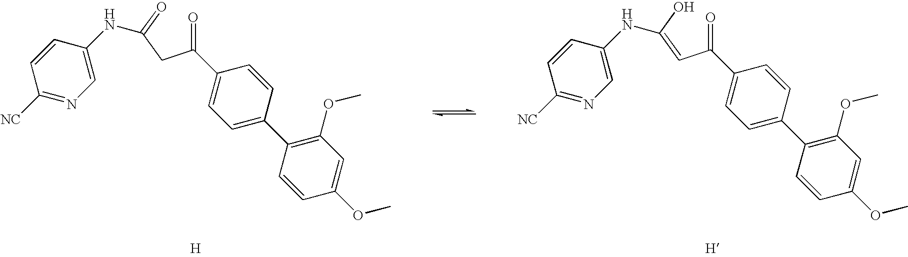
Preparation of Intermediate H.

Intermediate H was prepared by analogy to the method used to prepare compound 1a. ¹H NMR (acetone-d6) δ 8.86 (dd, 1H), 8.42 (m, 1H), 8.05 (d, 1.3 H), 7.90 (dd, 1H), 7.82 (d, 1H), 7.67 (d, 1.3 H), 7.61 (d, 0.8 H), 7.30 (dd, 1H), 6.65 (m, 2H), 6.07 (s, 0.3 H), 4.30 (s, 1 H), 7.27 (d, 1H), 6.66 (m, 2H), 6.41 (s, 1H), 3.80 (s, 3H), 3.78 (s, 3H)
Preparation of Intermediate I. 5{[3-(2′,4′-dimethoxy-1,1′-biphenyl-4-yl)-1H-pyrazol-5-yl]amino}-2-cyanopyridine

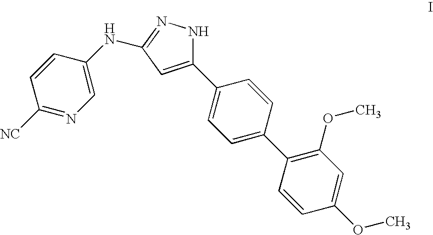

Intermediate I was prepared by analogy to compound 1b. ¹H NMR (acetone-d₆) δ 8.18 (m, 1H), 8.70 (m, 1H), 7.73 (d, 2H), 7.71 (d, 1H), 7.58 (d, 2H), 7.28 (d, 1H), 6.67 (b, 1H), 6.63 (dd, 1H), 6.40 (s, 1H), 3.84 (s, 3H), 3.82 (s, 3H).
Preparation of Intermediate G. 5{[3-(2′,4′-dimethoxy-1,1′-biphenyl-4-yl)-1H-pyrazol-5-yl]amino}pyridine-2-carbaldehyde

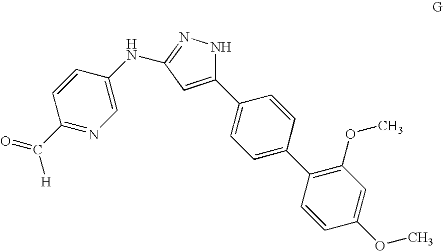

The aldehyde intermediate G was prepared from the cyano precursor as follows. Into a solution of 5-{[3-(2′,4′-dimethoxy-1,1′-biphenyl-4-yl)-1H-pyrazol-5-yl]amino}-2-cyanopyridine (750 mg, 1.88 mmol) in THF 30 mL, DIBAL 1.5 M in toluene (1.4 mL, 2.07 mmol) was added at −20° C. under N₂. The mixture was stirred at −20° C. for 4 hours continuously. Methanol was added to the reaction mixture to quench the reaction and 1N HCl was used to adjust pH 4-5. The mixture was diluted with ethyl acetate and washed with H₂O for two times. Organic layer was separated and dried over Na₂SO₄. Solvent was removed under reduced pressure. The resulting residue was recrystallization from dichloromethane and hexanes to afford 5-{[3-(2′,4′-dimethoxy-1,1′-biphenyl-4-yl)-1H-pyrazol-5-yl]amino}pyridine-2-carbaldehyde as a brown solid 327 mg (yield 43%). It was used to next step without further purification. ¹H NMR (DMSO-D6) δ 12.84 (s, 1H), 9.78 (s, 1H), 9.67 (s, 1H), 8.73 (s, 1H), 8.02 (d, 1H), 7.85 (d, 1H), 7.76 (d, 2H), 7.53 (d, 2H), 7.27 (d, 1H), 6.66 (m, 2H), 6.41 (s, 1H), 3.80 (s, 3H), 3.78 (s, 3H).

### General Procedure for the Synthesis of Examples 23-26

The compounds of examples 23-26 were synthesized according to Figure VI using the following procedure. A mixture of carbonyl compound G (1 mmol) and the amine (1-3 mmol) dissolved in DCE, or THF, or DCM was treated with sodium triacetoxyborohydride (1.4-1.5 mmol) under a nitrogen atmosphere at room temperature for 0.5-96 hours. The resulted mixture was purified through Prep-HPLC to afford the desired compound.

### Example 23

### Preparation of Compound 23

A. Preparation of Intermediate 23a. 6-{[(cyclopropylmethyl)amino]methyl}-N-[3-(2′,4′-dimethoxy-1,1′-biphenyl-4-yl)-1H-pyrazol-5-yl]pyridin-3-amine

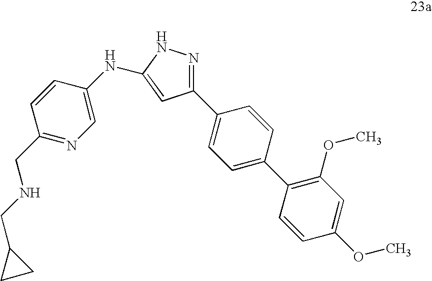

Compound 23a was prepared according to Figure VI. ¹H NMR (methanol-d₄) d 8.54 (d, 1H), 7.78 (q, 1H), 7.67 (d, 2H), 7.53 (d, 2H), 7.31 (d, 1H), 7.24 (d, 1H), 6.63 (m, 2H), 6.28 (s, 1H), 4.05 (s, 2H), 3.83 (s, 3H), 3.80 (s, 3H), 2.74 (d, 2H), 1.06 (m, 1H), 0.63 (q, 2H), 0.30 (q, 2H).
B. Preparation of

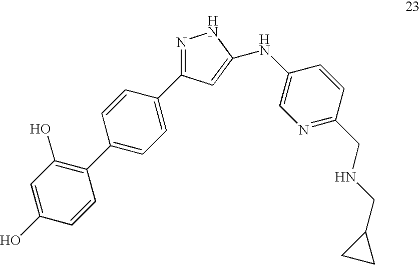

Example 23 was prepared according to Figure II. ¹H NMR (methanol-d₄) δ 8.56 (d, 1H), 7.83 (q, 1H), 7.66 (d, 2H), 7.61 (d, 2H), 7.31 (d, 1H), 7.12 (d, 1H), 6.39 (m, 2H), 6.27 (s, 1H), 4.14 (s, 2H), 2.86 (d, 2H), 1.09 (m, 1H), 0.68 (q, 2H), 0.35 (q, 2H).

### Example 24

### Preparation of Compound 24

A. Preparation of Intermediate 24a. 6-[(cyclopropylamino)methyl]-N-[3-(2′,4′-dimethoxy-1,1′-biphenyl-4-yl)-1H-pyrazol-5-yl]pyridin-3-amine

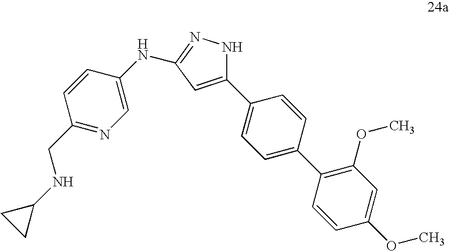

Intermediate 24a was prepared by analogy to 23a. ¹H NMR (methanol-d₄) δ 8.52 (d, 1H), 7.80 (q, 1H), 7.67 (d, 2H), 7.53 (d, 2H), 7.33 (d, 1H), 7.25 (d, 1H), 6.62 (m, 2H), 6.28 (s, 1H), 4.03 (s, 2H), 3.83 (s, 3H), 3.80 (s, 3H), 2.41 (m, 1H), 0.63 (m, 4H).
B. Preparation of

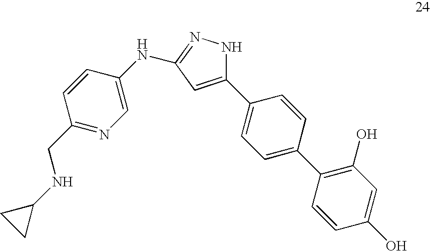

Compound 24 was prepared by analogy to compound 23. ¹H NMR (CD₃OD) δ 8.52 (d, 1H), 7.81 (q, 1H), 7.66 (d, 2H), 7.60 (d, 2H), 7.31 (d, 1H), 7.12 (d, 1H), 6.39 (m, 2H), 6.27 (s, 1H), 4.03 (s, 2H), 2.43 (s, 1H), 0.62 (m, 4H).

### Example 25

### Preparation of Compound 25

A. Preparation of Intermediate 25a. N-[3-(2′,4′-dimethoxy-1,1′-biphenyl-4-yl)-1H-pyrazol-5-yl]-6-[(isopropylamino)methyl]pyridin-3-amine

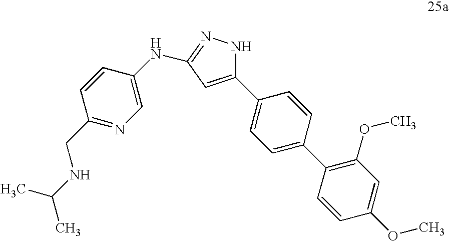

Intermediate 25a was prepared by analogy to the preparation of compound 24a. ¹H NMR (methanol-d₄) δ8.55 (d, 1H), 7.81 (q, 1H), 7.66 (d, 2H), 7.53 (d, 2H), 7.33 (d, 1H), 7.25 (d, 1H), 6.62 (m, 2H), 6.28 (s, 1H), 4.09 (s, 2H), 3.83 (s, 3H), 3.80 (s, 3H), 3.27 (m, 1H), 1.30 (d, 6H).
B. Preparation of

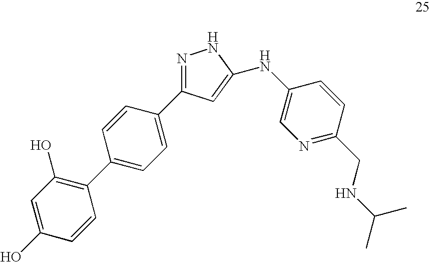

Compound 25 was prepared by analogy to compound 24. ¹H NMR (methanol-d₄) δ 8.55 (d, 1H), 7.83 (q, 1H), 7.66 (d, 2H), 7.61 (d, 2H), 7.32 (d, 1H), 7.12 (d, 1H), 6.38 (m, 2H), 6.27 (s, 1H), 4.13 (s, 2H), 3.31 (m, 1H), 1.34 (d, 6H).

### Example 26

### Preparation of Compound 26

A. Preparation of Intermediate 26a. N-[3-(2′,4′-dimethoxy-1,1′-biphenyl-4-yl)-1H-pyrazol-5-yl]-6-[(ethylamino)methyl]pyridin-3-amine

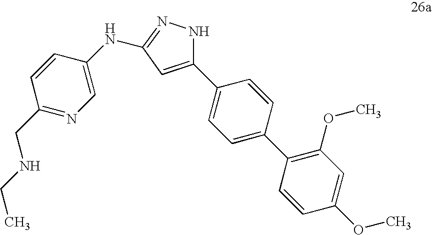

Intermediate 26a was prepared by analogy to compound 23a. ¹H NMR (methanol-d4) δ 8.56 (d, 1H), 7.83 (q, 1H), 7.67 (d, 2H), 7.54 (d, 2H), 7.32 (d, 1H), 7.25 (d, 1H), 6.62 (m, 2H), 6.28 (s, 1H), 4.08 (s, 2H), 3.84 (s, 3H), 3.80 (s, 3H), 3.00 (q, 2H), 1.29 (t, 3H).
Preparation of

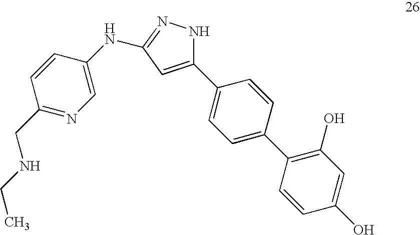

Compound 26 was prepared by analogy to compound 25. ¹H NMR (CD₃OD) δ 8.56 (d, 1H), 7.83 (q, 1H), 7.67 (d, 2H), 7.54 (d, 2H), 7.32 (d, 1H), 7.25 (d, 1H), 6.62 (m, 2H), 6.28 (s, 1H), 4.08 (s, 2H), 3.00 (q, 2H), 1.29 (t, 3H).

### EXAMPLES OF PHARMACEUTICAL COMPOSITIONS

### Example P1

### Parenteral Composition

To prepare a parenteral pharmaceutical composition suitable for administration by injection, 100 mg of a water-soluble salt of a compound of Formula (I) is dissolved in DMSO and then mixed with 10 mL of 0.9% sterile saline. The mixture is incorporated into a dosage unit form suitable for administration by injection.

### Example P2

### Oral Composition

To prepare a pharmaceutical composition for oral delivery, 100 mg of a compound of Formula (I) is mixed with 750 mg of lactose. The mixture is incorporated into an oral dosage unit for, such as a hard gelatin capsule, which is suitable for oral administration.

### Example P3

### Intraocular Composition

To prepare a sustained-release pharmaceutical composition for intraocular delivery, a compound of Formula (I) is suspended in a neutral, isotonic solution of hyaluronic acid (1.5% conc.) in phosphate buffer (pH 7.4) to form a 1% suspension.

Representative compounds of the present invention were tested against other kinases as well, i.e. CHK2; PKC-α; c-SRC; ERK2; GST-LCK; PLK and CDK2. The results showed that amino pyrazole compounds of the present invention were at least 20-fold more selective for CHK1 than for other kinases.

### BIOLOGICAL TESTING; ENZYME ASSAYS; SELECTION OF ACTIVE COMPOUNDS

### Example A

### CHK1 Assay (Table I)

### FL-CHK1 Construct for Assay

C-terminally His-tagged full-length human CHK1 (FL-CHK1) was expressed using the baculovirus/insect cell system. It contains 6 histidine residues (6 x His-tag) at the C-terminus of the 476 amino acid human CHK1. The protein was purified by conventional chromatographic techniques.

### FL-CHK1 Assay

The production of ADP from ATP that accompanies phosphoryl transfer to the synthetic substrate peptide Syntide-2 (PLARTLSVAGLPGKK) was coupled to oxidation of NADH using phosphoenolpyruvate (PEP) through the actions of pyruvate kinase (PK) and lactic dehydrogenase (LDH). The oxidation of NADH was monitored by following the decrease of absorbance at 340 nm (ε340=6.22 cm⁻¹ mM⁻¹) using a HP8452 spectrophotometer. Typical reaction solutions contained: 4 mN PEP; 0.15 mM NADH; 28 units of LDH/mL; 16 units of PK/mL; 3 mM DTT; 0.125 mM Syntide-2; 0.15 mM ATP; 25 mM MgCl₂ in 50 mM TRIS, pH 7.5; and 400 mM NaCl. Assays were initiated with 10 nM of FL-CHK1. Kᵢ values were determined by measuring initial enzyme activity in the presence of varying concentrations of test compounds. The data were analyzed using Enzyme Kinetic and Kaleidagraph software.

The results of the assays are presented in Table I. Certain compounds of Formula (I) exhibited a selectivity for CHK1 over other kinases that were tested. In some cases, selectivity for CHK1 exceeded selectivity for other tested kinases by at least a factor of 10.

### Kinase Domain of Human CHK-1 (KH-289) Construct for Assay

As previously detailed in European Patent Application No. 1 096 014 A2 (filed Oct. 31, 2000), the C-terminally His-tagged kinase domain of human CHK-1 (KH289), amino acid residues 1-289, can be expressed using the baculovirus/insect cell system. This construct has been shown to possess catalytic activity approximately 10-fold greater than full length CHK-1. The Bac-to-Bac system (Life Technologies) can be used to generate recombinant baculovirus for the expression of KH289 as per instructions. Recombinant viruses can be confirmed by PCR for the presence of CHK-1 cDNA insertion. Protein expression can be confirmed by SDS-PAGE or Western blot with CHK-1 polyclonal antibodies. Sf9 insect cells (Invitrogen, Carlsbad, Calif. USA) can be used for initial amplification of recombinant virus stock. High titer stocks of recombinant viruses can be generated by 2 to 3 rounds of amplification using Sf21 insect cells. Hi-S insect cells (Invitrogen, Carlsbad, Calif. USA) can be used for protein production. Both Sf9 and Hi-S cell lines can be adapted to grow in insect medium containing 1% Fetal Bovine Serum (Life Technologies, Grand Island, N.Y., USA). The viral stock was stored at 10° C. and used for large-scale protein production within 2 months to avoid viral instability. For protein production, infected Hi-S cells can be harvested by centrifugation and stored at −80° C. From these cells, 6X-His tagged KH289 (identified by SDS-PAGE) can be obtained after purification and can be flash-frozen in liquid N₂ and stored at −80° C. Maintaining salt concentration around 500 mM NaCl including 5% glycerol was found to be crucial for preventing aggregation of CHK-1 proteins during purification and storage.

### KH-289 CHK-1 Assay

As previously detailed in European Patent Application No. 1 096 014 A2 (filed Oct. 31, 2000), the enzymatic activity of a kinase can be measured by its ability to catalyze the transfer of a phosphate residue from a nucleoside triphosphate to an amino acid side chain in a selected protein target. The conversion of ATP to ADP generally accompanies the catalytic reaction. Herein, a synthetic substrate peptide, Syntide-2, having amino acid sequence PLARTLSVAGLPGKK can be utilized. The production of ADP from ATP that accompanies phosphoryl transfer to the substrate can be coupled to oxidation of NADH using phosphoenolpyruvate (PEP) through the actions of pyruvate kinase (PK) and lactic dehydrogenase (LDH). The oxidation of NADH can be monitored by following the decrease of absorbance at 340 nm (e340=6.22 cm−1 mM−1) using a HP8452 spectrophotometer. Typical reaction solutions contained: 4 mM PEP, 0.15 mM NADH, 28 units of LDH/mL, 16 units of PK/mL, 3 mM DTT, 0. 125 mM Syntide-2, 0.15 mM ATP and 25 mM MgCl2 in 50 mM TRIS pH 7.5; 400 mM NaCl. Assays can be initiated with 10 nM of kinase domain of CHK-1, KH289. Ki values can be determined by measuring initial enzyme activity in the presence of varying concentrations of inhibitors. The data can be analyzed using Enzyme Kinetic and Kaleidagraph software.

### Example B

### VEGF-R2

### VEGF-R2 Construct for Assay

This construct determines the ability of a test compound to inhibit tyrosine kinase activity. A construct (VEGF-R2Δ50) of the cytosolic domain of (human) vascular endothelial growth factor receptor 2 (VEGF-R2) lacking the 50 central residues of the 68 residues of the kinase insert domain can be expressed in a baculovirus/insect cell system. Of the 1356 residues of full-length VEGF-R2, VEGF-R2Δ50 contains residues 806-939 and 990-1171, and also one point mutation (E990V) within the kinase insert domain relative to wild-type VEGF-R2. Autophosphorylation of the purified construct can be performed by incubation of the enzyme at a concentration of 4 μM in the presence of 3 mM ATP and 40 mM MgCl₂ in 100 mM HEPES, pH 7.5, containing 5% glycerol and 5 mM DTT, at 4° C. for 2 hours. After autophosphorylation, this construct has been shown to possess catalytic activity essentially equivalent to the wild-type autophosphorylated kinase domain construct. See Parast et al. (1998) Biochemistry 37:16788-16801.

### VEGF-R2 Assay

a) Coupled Spectrophotometric (FLVK-P) Assay

The production of ADP from ATP that accompanies phosphoryl transfer can be coupled to oxidation of NADH using phosphoenolpyruvate (PEP) and a system having pyruvate kinase (PK) and lactic dehydrogenase (LDH). The oxidation of NADH can be monitored by following the decrease of absorbance at 340 nm (e₃₄₀=6.22 cm⁻¹ mM⁻¹) using a Beckman DU 650 spectrophotometer. Assay conditions for phosphorylated VEGF-R2Δ50 can be the following: 1 mM PEP; 250 μM NADH; 50 units of LDH/mL; 20 units of PK/mL; 5 mM DTT; 5.1 mM poly(E₄Y₁); 1 mM ATP; and 25 mM MgCl₂ in 200 mM HEPES, pH 7.5. Assay conditions for unphosphorylated VEGF-R2Δ50 can be the following: 1 mM PEP; 250 μM NADH; 50 units of LDH/mL; 20 units of PK/mL; 5 mM DTT; 20 mM poly(E₄Y₁); 3 mM ATP; and 60 mM MgCl₂ and 2 mM MnCl₂ in 200 mM HEPES, pH 7.5. Assays can be initiated with 5 to 40 nM of enzyme. Enzyme percentage inhibition values can be determined by measuring enzyme activity in the presence of 0.05 μM test compound. The data can be analyzed using Enzyme Kinetic and Kaleidagraph software.

### Example C

### FGFR

### FGF-R1 Construct for Assay

The intracellular kinase domain of (human) FGF-R1 can be expressed using the baculovirus vector expression system starting from the endogenous methionine residue 456 to glutamate 766, according to the residue numbering system of Mohammadi et al. (1996) Mol. Cell. Biol. 16:977-989. In addition, the construct also has the following 3 amino acid substitutions: L457V, C488A, and C584S.

### FGF-R Assay

The spectrophotometric assay can be carried out as described above for VEGF-R2, except for the following changes in concentration: FGF-R=50 nM, ATP=2 mM, and poly(E4Y1)=15 mM. Kᵢ values can be determined by measuring enzyme activity in the presence of varying concentrations of test compounds.

### Example D

### PHK

### PhosDhorylase Kinase Construct for Assay

The truncated catalytic subunit (gamma subunit) of phosphorylase kinase (amino acids 1-298) can be expressed in E. coli and isolated from inclusion bodies. Phosphorylase kinase can then be refolded and stored in glycerol at −20° C.

### Phosphorylase Kinase Assay

In the assay, the purified catalytic subunit can be used to phosphorylate phosphorylase b using radiolabled ATP. Briefly, 1.5 mg/ml of phosphorylase b can be incubated with 10 nM phosphorylase kinase in 10 mM MgCl₂, 50 mM Hepes pH 7.4, at 37° C. The reaction can be started with the addition of ATP to 100 uM and incubated for 15 min at 25° C. or 37° C. The reaction can be terminated and proteins can be precipitated by the addition of TCA to 10% final concentration. The precipitated proteins can be isolated on a 96 well Millipore MADP NOB filter plate. The filter plate can be extensively washed with 20% TCA, and dried. Scintillation fluid can be then added to the plate and incorporated radiolabel can be counted on a Wallac microbeta counter. The % inhibition of phosphoryl transfer from ATP to phosphorylase b in the presence of 10 μM of compound can then be measured.

### Example E

### Other Kinase Assays

### CHK-2 Assay

CHK-2 enzyme can be obtained from Upstate Group, Inc. and is an N-terminal, GST-tagged and C-terminal His-tagged fusion protein corresponding to amino acids 5-543 of human CHK-2 as confirmed by mass tryptic fingerprinting, expressed in E. coli; Mr˜87 kDa. The assay condition for CHK-2 can be as described above for CHK-1, except that the enzyme CHK2 (0.059 μM) can be utilized in place of KH289. Further, no NaCl can be added.

### CDK-1 Assay

CDK-1/cyclin B, active complex can be obtained from Upstate Group, Inc. and is a C-terminal, His-tagged CDK-1 and an N-terminal GST-tagged-cyclin B as confirmed by mass tryptic fingerprinting and protein sequencing, produced individually in Sf21 cells and then complexed in vitro. The assay condition for CDK-1 can be as described above for CHK-1, except that the enzyme complex CDK-1/cyclin B (0.2 μM) can be utilized in place of KH289, and Histone-H1 (Upstate USA, Inc.) (0.059 μM) can be utilized as a substrate in place of Syntide-2. Further, no NaCl can be added.

### WEE-1 Assay

### Delfia® Assay Protocol for WEE-1

WEE-1 enzyme can be obtained from Upstate Group, Inc. and is an N-terminal, GST-tagged fusion protein to full length rat WEE-1, expressed in E. coli; Mr˜100 kDa. This kinase assay can be carried out on coated poly (Glu-Tyr) 4:1 (random copolymer) 96-well filter plates (NoAb Diagnostics). The assay volume can be 100 μl per well plus 2 μl DMSO (control) or 2 μl of compound in DMSO. Buffer A can be 10% glycerol, 20 mM TRIS (pH7.5), 10 mM MgCl₂, 50 mM NaCl and 5 mM DTT. The plates can be prepared by automation.

To an appropriate well can be added either 2 μl of DMSO (control) or 2 μl of compound in DMSO. To the positive control wells can be added 30 μl of 0.5M EDTA. To each well can be added 50 μl ATP in Buffer A such that the ATP assay concentration can be 33 μM. To start the reaction, 50 μl Wee1 in Buffer A can be added to each well such that the Wee1 assay concentration can be 0.1 ng/μg. The plate can be can be mixed by shaking and then allowed to remain at room temperature for 30 minutes. To stop the reaction, the plate can be washed once with Delfia Wash on an EL405 plate washer. To each well can be added 100 μl of EuPY in Delfia® assay buffer such that the EuPY assay concentration can be 0.0149 ng/μl. The plate can be allowed to sit for 1 hours or overnight. The plate can be washed once again with Delfia® Wash (EL405 plate washer), and allowed to dry. To each well can be added 100 μl of Delfia® Enhancement solution and the plate can be allowed to sit for 10 minutes. The plate can be read on Wallac's Victor fluorescence reader (Europium Protocol). Kᵢ values can be determined by measuring enzyme activity in the presence of varying concentrations of test compounds.

### SGK Assay

SGK (human) (Upstate Group, Inc., KINASEPROFILER™) (5-10 mU) can be incubated with 8 mM MOPS pH7.0, 0.2 mM EDTA, 30 μM Crosstide, 10 mM MgAcetate and [γ-³³P-ATP] (Specific activity approximately 500 cpm/pmol, concentration as required) to form a final reaction volume of 25 μl. Compounds can be tested at 1 μM. The reaction can be initiated by the addition of Mg²⁺ [γ-³³P-ATP]. The ATP concentration can be 10 μM. After incubation for 40 minutes at room temperature, the reaction can be stopped by the addition of 5 μl of a 3% phosphoric acid solution. 10 μl of the reaction can then be spotted onto a P30 filtermat and washed three times for 5 minutes in 50 mM phosphoric acid and once in methanol prior to drying and scintillation counting. Results represent an average of two experiments and enzymatic activity can be expressed as a percentage of that in control incubations without test compounds.

### AMPK Assay

AMPK (rat) (Upstate Group, Inc., KINASEPROFILER™) (5-10 mU) can be incubated with 5 mM Hepes pH 7.4, 1 mM DTT, 0.02% Brij35, 200 μM AMP, 200 μM AMARAASAAALARRR, 10 mM MgAcetate and [γ-³³P-ATP] (Specific activity approximately 500 cpm/pmol, concentration as required) to form a final reaction volume of 25 μl. Compounds can be tested at 1 μM. The reaction can be initiated by the addition of Mg²⁺ [γ-³³P-ATP]. The ATP concentration can be 10 μM. After incubation for 40 minutes at room temperature, the reaction can be stopped by the addition of 5 μl of a 3% phosphoric acid solution. 10 μl of the reaction can then be spotted onto a P30 filtermat and washed three times for 5 minutes in 75 mM phosphoric acid and once in methanol prior to drying and scintillation counting. Results represent an average of two experiments and enzymatic activity can be expressed as a percentage of that in control incubations without test compound.

### LCK Assay

LCK (human) (Upstate Group, Inc., KINASEPROFILER™) (5-10 mU) can be incubated with 50 mM Tris pH7.5, 0.1 mM EGTA, 0.1 mM NaVanadate, 250 μM KVEKIGEGTYGVVYK (CDC2 peptide), 10 mM MgAcetate and [γ³³P-ATP] (Specific activity approximately 500 cpm/pmol, concentration as required) to form a final reaction volume of 25 μl. Compounds can be tested at 1 μM. The reaction can be initiated by the addition of Mg²⁺ [γ³³P-ATP]. The ATP concentration can be 10 μM. After incubation for 40 minutes at room temperature, the reaction can be stopped by the addition of 5 μl of a 3% phosphoric acid solution. 10 μl of the reaction can then be spotted onto a P30 filtermat and washed three times for 5 minutes in 75 mM phosphoric acid and once in methanol prior to drying and scintillation counting. Results represent an average of two experiments and enzymatic activity can be expressed as a percentage of that in control incubations without test compound.

### MAPK2 Assay

MAPK2 (mouse) (Upstate Group, Inc., KINASEPROFILER™) (5-10 mU) can be incubated with 25 mM Tris pH 7.5, 0.02 mM EGTA, 0.33 mg/ml myelin basic protein, 10 mM MgAcetate and [γ-³³P-ATP] (Specific activity approximately 500 cpm/pmol, concentration as required) to form a final reaction volume of 25 μl. Compounds can be tested at 1 μM. The reaction can be initiated by the addition of Mg²⁺ [γ-³³P-ATP]. The ATP concentration can be 10 μM. After incubation for 40 minutes at room temperature, the reaction can be stopped by the addition of 5 μl of a 3% phosphoric acid solution. 10 μl of the reaction can then be spotted onto a P30 filtermat and washed three times for 5 minutes in 75 mM phosphoric acid and once in methanol prior to drying and scintillation counting. Results represent an average of two experiments and enzymatic activity can be expressed as a percentage of that in control incubations without test compound.

### MSK1 Assay

MSK1 (human) (Upstate Group, Inc., KINASEPROFILER™) (5-10 mU) can be incubated with 8 mM MOPS pH7.0, 0.2 mM EDTA, 30 pM Crosstide, 10 mM MgAcetate and [γ-³³P-ATP] (Specific activity approximately 500 cpm/pmol, concentration as required) to form a final reaction volume of 25 μl. Compounds can be tested at 1 μM. The reaction can be initiated by the addition of Mg²⁺ [γ-³³P-ATP]. The ATP concentration can be 10 μM. After incubation for 40 minutes at room temperature, the reaction can be stopped by the addition of 5 μl of a 3% phosphoric acid solution. 10 μl of the reaction can then be spotted onto a P30 filtermat and washed three times for 5 minutes in 50 mM phosphoric acid and once in methanol prior to drying and scintillation counting. Results represent an average of two experiments and enzymatic activity can be expressed as a percentage of that in control incubations without test compound.

### PKBα Assay

PKBα (human) (Upstate Group, Inc., KINASEPROFILER™) (5-10 mU) can be incubated with 8 mM MOPS pH7.0, 0.2 mM EDTA, 30 μM Crosstide, 10 mM MgAcetate and [γ-³³P-ATP] (Specific activity approximately 500 cpm/pmol, concentration as required) to form a final reaction volume of 25 μl. Compounds can be tested at 1 μM. The reaction can be initiated by the addition of Mg²⁺ [γ-³³P-ATP]. The ATP concentration can be 10 μM. After incubation for 40 minutes at room temperature, the reaction can be stopped by the addition of 5 μl of a 3% phosphoric acid solution. 10 ul of the reaction can then be spotted onto a P30 filtermat and washed three times for 5 minutes in 50 mM phosphoric acid and once in methanol prior to drying and scintillation counting. Results represent an average of two experiments and enzymatic activity can be expressed as a percentage of that in control incubations without test compound.

### ROCKII Assay

ROCKII (rat) (Upstate Group, Inc., KINASEPROFILER™) (5-10 mU) can be incubated with 50 mM Tris pH7.5, 0.1 mM EGTA, 30 μM KEAKEKRQEQIAKRRRLSSLRASTSKSGGSQK, 10 mM MgAcetate and [ψ-³³P-ATP] (Specific activity approximately 500 cpm/pmol, concentration as required) to form a final reaction volume of 25 μl. Compounds can be tested at 1 μM. The reaction can be initiated by the addition of Mg²⁺ [γ-³³P-ATP]. The ATP concentration can be 10 μM. After incubation for 40 minutes at room temperature, the reaction can be stopped by the addition of 5 μl of a 3% phosphoric acid solution. 10 μl of the reaction can then be spotted onto a P30 filtermat and washed three times for 5 minutes in 75 mM phosphoric acid and once in methanol prior to drying and scintillation counting. Results represent an average of two experiments and enzymatic activity can be expressed as a percentage of that in control incubations without test compound.

### p70 S6K Assay

p70S6K (human) (Upstate Group, Inc., KINASEPROFILER™) (5-10 mU) can be incubated with 8 mM MOPS pH7.0, 0.2 mM EDTA, 100 μM KKRNRTLTV, 10 mM MgAcetate and [γ-³³P-ATP] (Specific activity approximately 500 cpm/pmol, concentration as required) to form a final reaction volume of 25 μl. Compounds can be tested at 1 μM. The reaction can be initiated by the addition of Mg²⁺ [γ-³³P-ATP]. The ATP concentration can be 10 μM. After incubation for 40 minutes at room temperature, the reaction can be stopped by the addition of 5 μl of a 3% phosphoric acid solution. 10 μl of the reaction can then be spotted onto a P30 filtermat and washed three times for 5 minutes in 75 mM phosphoric acid and once in methanol prior to drying and scintillation counting. Results represent an average of two experiments and enzymatic activity can be expressed as a percentage of that in control incubations without test compound.

### PKA Assay

PKA (bovine) (Upstate Group, Inc., KINASEPROFILER™) (5-10 mU) can be incubated with 8 mM MOPS pH7.0, 0.2 mM EDTA, 30 μM LRRASLG (Kemptide), 10 mM MgAcetate and [γ-³³P-ATP] (Specific activity approximately 500 cpm/pmol, concentration as required) to form a final reaction volume of 25 μl. Compounds can be tested at 1 μM. The reaction can be initiated by the addition of Mg²⁺ [γ-³³P-ATP]. The ATP concentration can be 10 μM. After incubation for 40 minutes at room temperature, the reaction can be stopped by the addition of 5 μl of a 3% phosphoric acid solution. 10 μl of the reaction can then be spotted onto a P30 filtermat and washed three times for 5 minutes in 50 mM phosphoric acid and once in methanol prior to drying and scintillation counting. Results represent an average of two experiments and enzymatic activity can be expressed as a percentage of that in control incubations without test compound.

### MAPK1 Assay

MAPK1 (human) (Upstate Group, Inc., KINASEPROFILER™) (5-10 mU) can be incubated with 25 mM Tris pH7.5, 0.02 mM EGTA, 1 mM synthetic peptide, 10 mM MgAcetate and [γ-³³P-ATP] (Specific activity approximately 500 cpm/prnol, concentration as required) to form a final reaction volume of 25 μl. Compounds can be tested at 1 μM. The reaction can be initiated by the addition of Mg²⁺ [γ-³³P-ATP]. The ATP concentration can be 10 μM. After incubation for 40 minutes at room temperature, the reaction can be stopped by the addition of 5 μl of a 3% phosphoric acid solution. 10 μl of the reaction can then be spotted onto a P30 filtermat and washed three times for 5 minutes in 75 mM phosphoric acid and once in methanol prior to drying and scintillation counting. Results represent an average of two experiments and enzymatic activity can be expressed as a percentage of that in control incubations without test compound.

### cSRC Assay

cSRC (human) (Upstate Group, Inc., KINASEPROFILER™) (5-10 mU) can be incubated with 8 mM MOPS pH7.0, 0.2 mM EDTA, 250 μM KVEKIGEGTYGVVYK (CDC2 peptide), 10 mM MgAcetate and [γ-³³P-ATP] (Specific activity approximately 500 cpm/pmol, concentration as required) to form a final reaction volume of 25 μl. Compounds can be tested at 1 μM. The reaction can be initiated by the addition of Mg²⁺ [γ-³³P-ATP]. The ATP concentration can be 10 μM. After incubation for 40 minutes at room temperature, the reaction can be stopped by the addition of 5 μl of a 3% phosphoric acid solution, 10 μl of the reaction can then be spotted onto a P30 filtermat and washed three times for 5 minutes in 75 mM phosphoric acid and once in methanol prior to drying and scintillation counting. Results represent an average of two experiments and enzymatic activity can be expressed as a percentage of that in control incubations without test compound.

### PRK2 Assay

PRK2 (human) (Upstate Group, Inc., KINASEPROFILER™) (5-10 mU) can be incubated with 50 mM Tris pH7.5, 0.1 mM EGTA, 0.1% β-mercaptoethanol, 30 μM AKRRRLSSLRA, 10 mM MgAcetate and [γ-³³P-ATP] (Specific activity approximately 500 cpm/pmol, concentration as required) to form a final reaction volume of 25 μl. Compounds can be tested at 1 μM. The reaction can be initiated by the addition of Mg²⁺ [γ-³³P-ATP]. The ATP concentration can be 10 μM. After incubation for 40 minutes at room temperature, the reaction can be stopped by the addition of 5 μl of a 3% phosphoric acid solution. 10 μl of the reaction can then be spotted onto a P30 filtermat and washed three times for 5 minutes in 75 mM phosphoric acid and once in methanol prior to drying and scintillation counting. Results represent an average of two experiments and enzymatic activity can be expressed as a percentage of that in control incubations without test compound.

### PDK1 Assay

PDK1 (human) (Upstate Group, Inc., KINASEPROFILER™) (5-10 mU) can be incubated with 50 mM Tris pH7.5, 100 μM KTFCGTPEYLAPEVRREPRILSEEEQEMFRDFDYIADWC (PDKtide), 0.1% β-mercaptoethanol, 10 mM MgAcetate and [γ-³³P-ATP] (Specific activity approximately 500 cpm/pmol, concentration as required) to form a final reaction volume of 25 μl. Compounds can be tested at 1 μM. The reaction can be initiated by the addition of Mg²⁺ [γ-³³P-ATP]. The ATP concentration can be 10 μM. After incubation for 40 minutes at room temperature, the reaction can be stopped by the addition of 5 μl of a 3% phosphoric acid solution. 10 μl of the reaction can then be spotted onto a P30 filtermat and washed three times for 5 minutes in 75 mM phosphoric acid and once in methanol prior to drying and scintillation counting. Results represent an average of two experiments and enzymatic activity can be expressed as a percentage of that in control incubations without test compound.

### FYN Assay

FYN (human) (Upstate Group, Inc., KINASEPROFILER™) (5-10 mU) can be incubated with 50 mM Tris pH7.5, 0.1 mM EGTA, 0.1 mM NaVanadate, 250 μM KVEKIOEGTYGVVYK (CDC2 peptide), 10 mM MgAcetate and [γ-³³P-ATP] (Specific activity approximately 500 cpm/pmol, concentration as required) to form a final reaction volume of 25 μl. Compounds can be tested at 1 μM. The reaction can be initiated by the addition of Mg²⁺ [γ-³³P-ATP]. The ATP concentration can be 10 μM. After incubation for 40 minutes at room temperature, the reaction can be stopped by the addition of 5 μl of a 3% phosphoric acid solution. 10 μl of the reaction can then be spotted onto a P30 filtermat and washed three times for 5 minutes in 75 mM phosphoric acid and once in methanol prior to drying and scintillation counting. Results represent an average of two experiments and enzymatic activity can be expressed as a percentage of that in control incubations without test compound.

### PKCβII Assay

PKCβII (human) (Upstate Group, Inc., KINASEPROFILER™) (5-10 mU) can be incubated with 20 mM Hepes pH7.4, 0.03% Triton X-100, 0.1 mM CaCl₂, 0.1 mg/ml phosphatidylserine, 10 μg/ml diacylglycerol, 0.1 mg/ml histone H1, 10 mM MgAcetate and [γ-³³P-ATP] (Specific activity approximately 500 cpm/pmol, concentration as required) to form a final reaction volume of 25 μl . Compounds can be tested at 1 μM. The reaction can be initiated by the addition of Mg²⁺ [γ-³³P-ATP]]. The ATP concentration can be 10 μM. After incubation for 40 minutes at room temperature, the reaction can be stopped by the addition of 5 μl of a 3% phosphoric acid solution. 10 μl of the reaction can then be spotted onto a P30 filtermat and washed three times for 5 minutes in 75 mM phosphoric acid and once in methanol prior to drying and scintillation counting. Results represent an average of two experiments and enzymatic activity can be expressed as a percentage of that in control incubations without test compound.

### PKCγ Assay

PKCγ (human) (Upstate Group, Inc., KINASEPROFILER™) (5-10 mU) can be incubated with 20 mM Hepes pH7.4, 0.03% Triton X-100, 0.1 mM CaCl₂, 0.1 mg/ml phosphatidylserine, 10 μg/ml diacylglycerol, 0.1 mg/ml histone H1, 10 mM MgAcetate and [γ-³³P-ATP] (Specific activity approximately 500 cpm/pmol, concentration as required) to form a final reaction volume of 25 μl. Compounds can be tested at 1 μM. The reaction can be initiated by the addition of Mg²⁺[γ-³³P-ATP]. The ATP concentration can be 10 μM. After incubation for 40 minutes at room temperature, the reaction can be stopped by the addition of 5 μl of a 3% phosphoric acid solution. 10 μl of the reaction can then be spotted onto a P30 filtermat and washed three times for 5 minutes in 75 mM phosphoric acid and once in methanol prior to drying and scintillation counting. Results represent an average of two experiments and enzymatic activity can be expressed as a percentage of that in control incubations without test compound.

### Example F

### Whole Cell Checkpoint Abrogation Assay

### CHK1 Mitotic Index ELISA Assay

To examine the in vitro effects of CHK1 inhibitory compounds, an ELISA assay can be designed to monitor the abrogation of DNA damage-induced checkpoint control. The assay can be based on the trapping and detection of mitotic cells following DNA damage-induced arrest. Phosphorylation of Histone H3 on serine 10 has been shown to correlate with mitosis and therefore can be required for chromosome condensation; consequently a mitosis specific phospho-epitope on Histone H3 can be used as a signal for checkpoint abrogation.

CA-46 (lymphoma) cells can be treated with a DNA damaging agent, such as camptothecin (Sigma), at 50 nM for 8 hours to induce DNA damage. The control compound or CHK1 inhibitor can be then added at increasing concentrations with Nocodazole (Sigma) at 0.1 μg/ml and plates can be incubated for 16 hours. Control cells, where only CHK1 inhibitors can be added, can be prepared as well to assure that the inhibitors alone have no effect on the cell cycle. The cells can be then harvested, washed with PBS, and crude acid extraction can be performed. Pellets can be resuspended in 80 μl of Acid Extraction Buffer (10 mM Hepes pH 7.9, 1.5 mM MgCl₂, 10 mM KCl, 0.5 mM DTT, 1.5 mM PMSF, 0.4N sulfuric acid), vortexed briefly, and incubated for 30 minutes on ice. Samples can be then centrifuged and 75 μl of the supernatant can be transferred to a 96 well flat-bottom plate (VWR 3596). Next 15 μl Neutralizing Cocktail (# of samples×(10 μl 10N NaOH+5 μl 1M Tris Base) can be added to each well, and after mixing, 5 μl of this can be transferred to another 96 well plate with 100 μl 50 mM Tris base (pH 9.6) in each well. Samples can be dried overnight. The wells can be then washed with 200 μl ELISA wash buffer (PBS with 20 mM Tris pH 7.5, 0.05% Tween 20) 5 times and blocked with 200 μl blocking buffer (PBS with 20 mM Tris pH 7.5, 0.05% Tween 20, 3.5% Dry milk, 1.5% BSA. pH to 7.5 after preparation) for 1 hour at room temperature. Following wash and block, anti-phospho Histone H3 antibodies (Upstate USA, Inc., rabbit polyclonal) can be added at 0.5 μg/ml in block (100 μl per well) and incubated for 2 hours at room temperature. Wells can be washed again to remove unbound primary antibody and 100 μl alkaline phosphatase conjugated secondary antibodies at 0.3 mg/ml (Pierce, goat anti-rabbit IgG (HOURS+L)) in block can be added for 1 hour at room temp. Wells can be washed 5 times to remove unbound secondary antibody, and washed again 3 times with PBS alone to remove detergents. Then 100 μl alkaline phosphatase substrate (Pierce 1-Step pNPP) can be added to wells. Plates can be protected from light and incubated at room temp for 1 hour. The OD can be read on Molecular Devices Vmax Kinetic Microplate Reader at 405 nm. The ratio of the OD (optical density) of a compound treated sample to the Nocodazole only treated sample (about 100% mitotic or abrogation) can be expressed in a percentage, and quantifies the percent abrogation of the checkpoint. The concentration at which a compound causes 50% abrogation of the checkpoint can be called the EC₅₀. The raw OD values can be graphed in Excel, and an EC₅₀ value can be generated using Kaleidograph software. Strong signal results from Nocodazole only treated cells, and equals 100% mitosis in this assay. Camptothecin+Nocodazole treated control samples have low signal, signifying no mitosis and therefore, no checkpoint abrogation. When potent CHK1 inhibitors are added to Camptothecin treated cells with Nocodazole, a high signal can be generated (generally in a dose dependent manner), due to the checkpoint abrogation activity caused by the combination treatment.

The examples above illustrate compounds according to Formula (I) and assays that may readily be performed to determine their activity levels against the various kinase complexes. For example, the selectivity of the compounds of Formula (I) for a kinase (e.g., CHK1) can be determined by comparing the ability of the compounds of Formula (I) to inhibit the kinases in the assays described above. In addition, and by way of example only, the ability of compound of Formula (I) to enhance the effect of a particular anti-neoplastic agent and/or DNA-damaging agent may determined by comparing the response of tumor cells to that anti-neoplastic agent and/or DNA-damaging agent in the presence and absence of a compound of Formula (I). A compound of Formula (I) that enhances the ability of the anti-neoplastic agent to destroy the tumor cells (either in number and/or response rate) and/or the ability of the DNA-damaging agent to damage DNA is preferred. It will be apparent that such assays or other suitable assays known in the art may be used to select an inhibitor having a desired level of activity against a selected target.

Representative compounds of the present invention were tested against other kinases as well, i.e. CHK2; PKC-α; c-SRC; ERK2; GST-LCK; PLK and CDK2. The results showed that aminopyrazole CHK1 compounds are at least 20-fold more selective for CHK1 than for other kinases.

### Example G

### CHKL Inhibitors Enhance Killing of Cells by Cancer Treatments

To test the hypothesis that inhibition of Chk-I potentiates the killing effect of DNA-damaging agents, cells can be incubated in the presence of selective ChkI inhibitors and either irradiation or 10 chemical DNA-damaging agents. Various cell lines (HT29, MV522, Colo205, etc.) were grown in 96-well plates. Cells were plated in the appropriate medium at a volume of 100 ul/well. Plates were incubated for four hours before the addition of inhibitor compounds. On the bottom part of the 96 well plate, cells were treated with increasing concentrations of DNA damaging agent. On the top part of the plate, cells were treated with increasing concentrations of DNA damaging agent combined with a fix concentration of the AG (inhibitor). Cells were incubated at 37° C. (5% CO₂) for four to six days (depending on cell type). At the end of the incubation, MTT was added to a final concentration of 0.2 mg/ml, and cells were incubated for 4 hours at 37° C. After centrifugation of the plates and removal of medium, the absorbance of the formazan (solubilized in dimethylsulfoxide) was measured at 540 nm. The concentrations of DNA damaging agent causing 50% growth inhibition in the presence and in the absence of the Chk1 inhibitor were determined from the linear portion of a semi-log plot of inhibitor concentration versus percent inhibition. The ratio between the IC50 of the agent alone and the IC50 of the combination treatment represents the PF50 (Potentiation Factor 50) and is a measure of the potency and effectiveness of the combination treatment.

All cell line designations refer to human cell lines and refer to the 20 following:

| | |
|---|---|
| HeLa | Cervical adenocarcinoma |
| ACHN | Renal adenocarcinoma |
| 786-0 | Renal adenocarcinoma |
| HCT116 | Colon carcinoma |
| SW620 | Colon carcinoma |
| HT-29 | Colonrectal adenocarcinoma |
| Colo205 | Colon adenocarcinoma |
| SK-MEL-5 | Melanoma |
| SK-MEL-28 | Malignant melanoma |
| A549 | Lung carcinoma |
| H322 | Brocholoalveolar carcinoma |
| OVCAR-3 | Ovarian adenocarcinoma |
| SK-OV-3 | Ovarian adenocarcinoma |
| MDA-MB-231 | Breast adenocarcinoma |
| MCF-7 | Breast adenocarcinoma |
| PC-3 | Prostate adenocarcinoma, from metastasis |
| HL-60 | Acute promyelocytic leukemia |
| K562 | Chronic myelogenous leukemia |
| MOLT4 | Acute lymphoblastic leukemia; T lymphoblast |

Chemotherapeutic drugs included etoposide, doxorubicin, cisplatin, chlorambucil, 5-fluorouracil (5-FU). At concentrations less than 0.5 uM, the test compounds of formula I enhanced the killing of cisplatin from 2- to 5-fold.

The compounds of Formula I can be tested with additional antimetabolites, including methotrexate, hydroxyurea, 2-chloroadenosine, fludarabine, azacytidine, and gemcitibine for an ability to enhance killing of the agents. At concentrations less than 0.5 uM, these ChkI inhibitors can be found to enhance the killing of cells to gemcitibine, hydroxyurea, fludarabine, 5-azacytidine, and methotrexate up to 10 fold, suggesting that the combination of inhibition of ChkI and blocking of DNA synthesis can lead to increased cell death by these agents. In addition, the ability of the ChkI inhibitor to enhance killing by irradiation can be tested. In HeLa cells, the test compounds of formula I were found to enhance killing by irradiation 2-3 fold.

### Example H

### Animal Tumor Models

Gemcitibine (Gemzar) is an antimetabolite that acts as a pyrimidine analog. To test the ability of the ChkI inhibitors to enhance the killing of tumors by Gemcitibine in mice, xenograft tumor models using colon tumor cell lines can be established. Co10205 and HT29 cells (human colon carcinoma) can be used to propagate xenograft tumors in 6-8 week old female thymic Balb/c (nu/nu) mice. Mice can be maintained in a laminar airflow cabinet under pathogen-free conditions and fed sterile food and water ad libitum. Cell lines can be grown to subconfluence in RPMI 1640 media supplemented with 10% FBS, 100 U/mL penicillin, 100 μg/mL streptomycin, and 1.5 mM L-glutamine in a 5% CO₂ humidified environment. Single cell suspensions can be prepared in CMF-PBS, and cell concentration adjusted to 1×10⁸ cells/mL. Mice can be inoculated subcutaneously (s.c). on the right flank or right leg with a total of 2×10⁶ cells (100 μL).

Mice can be randomized (12 mice/group) into treatment groups and used when tumors reach a weight of 150-200 mg (usually 7-11 days post-inoculation). The tumors can be measured with vernier calipers and tumor weights can be estimated using the empirically derived formula: tumor weight (mg)=tumor length (mm)×tumor width (mm)²/3.3. Treatment can consist of i) 100 μL intraperitoneal (i.p). injection of 5-FU at 50 mg/kg, 100 mg/kg, or 150 mg/kg. A dose-dependent delay in tumor growth can be observed in the mice treated with 5-FU. Tumor size can be monitored every other day for the duration of the experiment.

Obviously, many modifications and variations of the invention as hereinbefore set forth can be made without departing from the spirit and scope thereof, and, therefore, only such limitations should be imposed as are indicated by the appended claims.

TABLE I

| | | Ki (nM) | |
|---|---|---|---|
| | | <10 = A | |
| Example | | 1-10 = B | |
| Number | Structure | >1 = C | EC50 (nM) |
| 1 | 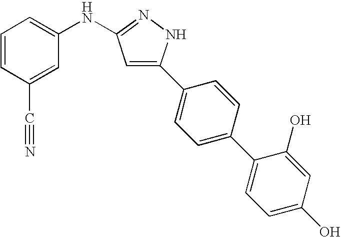 | B | 7% @ 0.5 μM |
| 2 | 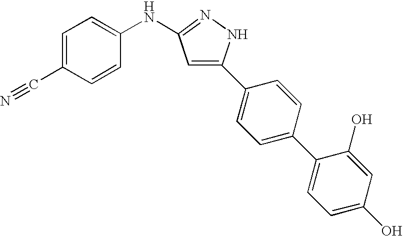 | B | 1% @ 1 μM |
| 3 | 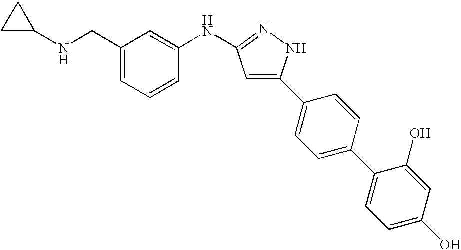 | C | 900 |
| 4 | 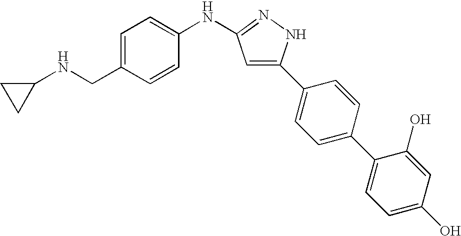 | C | 60 |
| 5 | 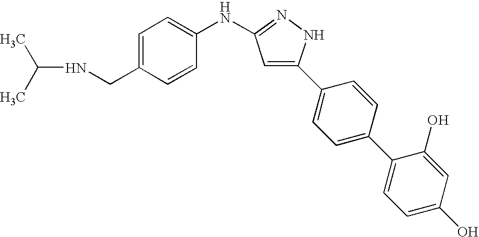 | C | 70 |
| 6 | 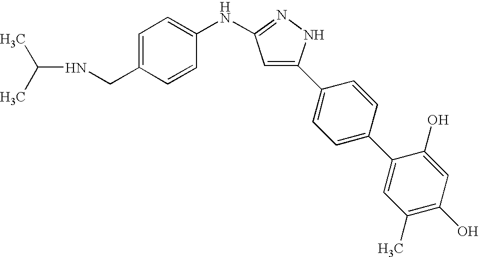 | A | Not available |
| 7 | 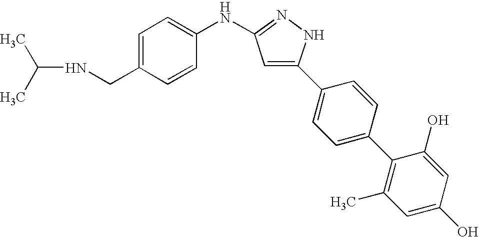 | C | 380 |
| 8 | 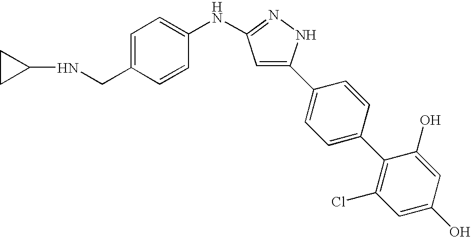 | C | 150 |
| 9 | 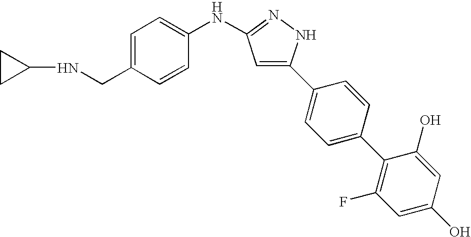 | B | 88 |
| 10 | 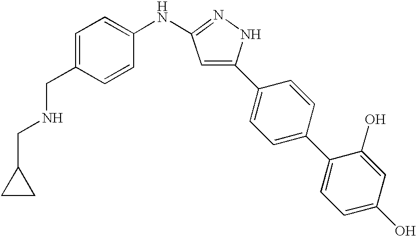 | C | 75 |
| 11 | 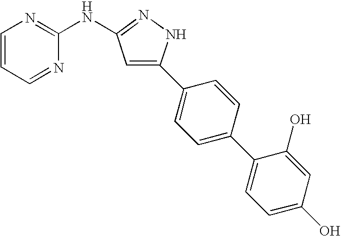 | B | Not available |
| 12 | 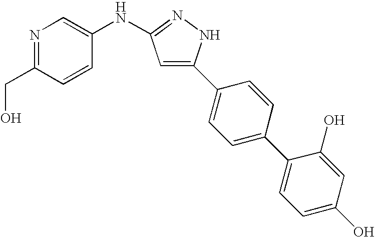 | A | 5% @ 0.5 μM |
| 13 | 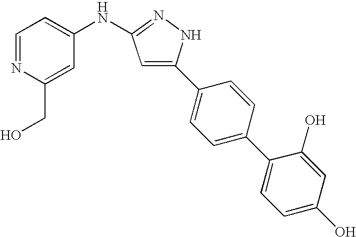 | C | 0% @ 0.5 μM |
| 14 | 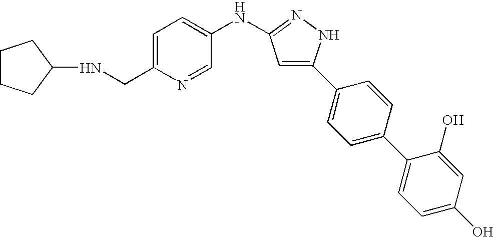 | B | 160 |
| 15 | 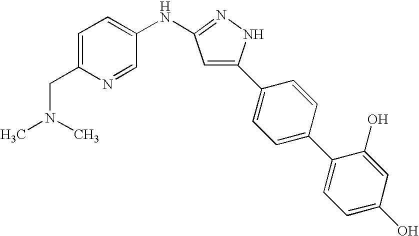 | B | 380 |
| 16 | 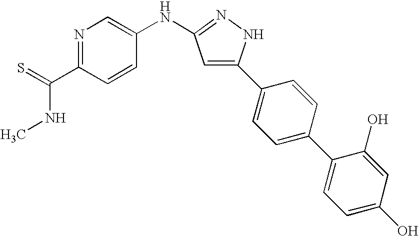 | B | 10% @ 0.5 μM |
| 17 | 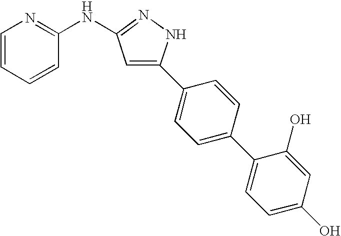 | C | 16% @ 0.5 μM |
| 18 | 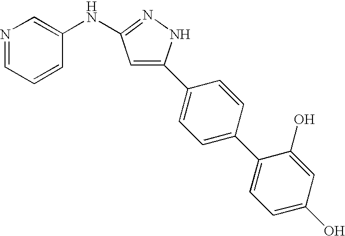 | C | 540 nM |
| 19 | 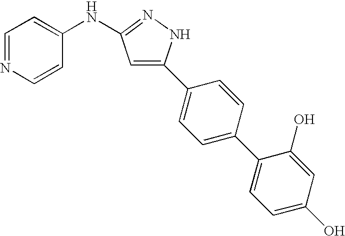 | B | 14% @ 0.5 μM |
| 20 | 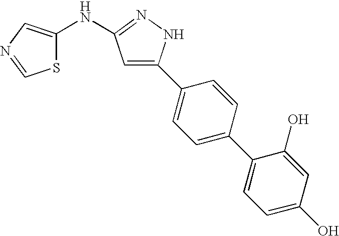 | C | 10% @ 0.5 μM |
| 21 | 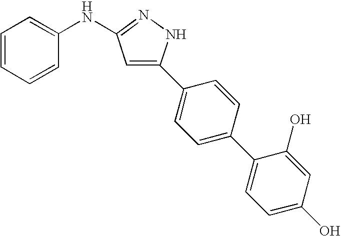 | C | 500 |
| 22 | 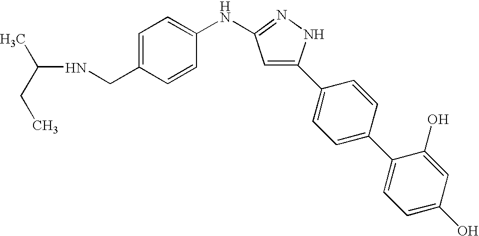 | C | 60 |
| 23 | 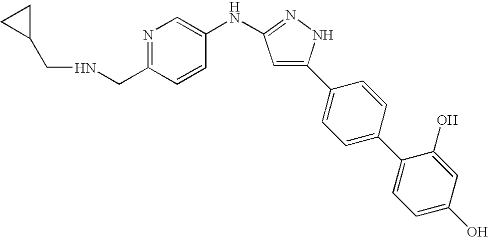 | B | 165 |
| 24 | 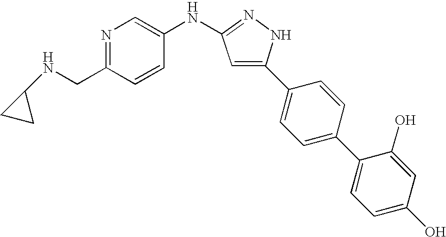 | B | 1% @ 1 μM |
| 25 | 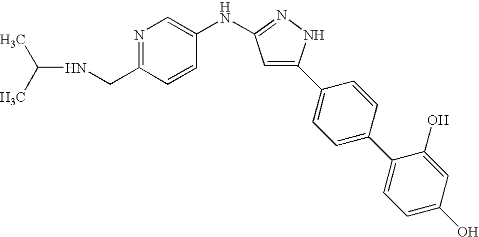 | C | 560 |
| 26 | 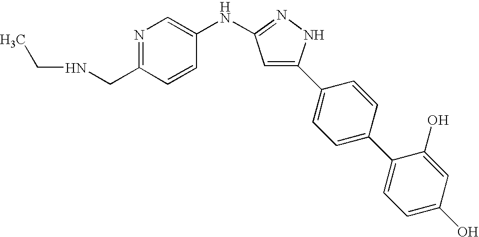 | C | 560 |

It is to be understood that the foregoing description is exemplary and explanatory in nature, and is intended to illustrate the invention and its preferred embodiments. Through routine experimentation, the artisan will recognize apparent modifications and variations that may be made without departing from the spirit of the invention. Thus, the invention is intended to be defined not by the above description, but by the following claims and their equivalents.

1. A compound having the structure of Formula (I): 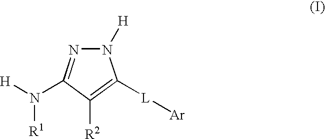
wherein L is a 5- or 6-membered carbocycle or heterocycle group, optionally substituted with 1-3 substituents independently selected from the group consisting of Y₁, Y₂ and Y₃;
Ar is a 5- or 6-membered aromatic carbocycle or heterocycle group, optionally substituted with 1-3 substituents independently selected from the group consisting of Y₁, Y₂ and Y₃;
R¹ is selected from the group consisting of —(CR³R⁴)ₜ-aryl, 13 (CR³R⁴)ₜ-heterocycle, —(CR³R⁴)ₜ—(C₃-C₆)cycloalkyl, (C₂-C₆)alkenyl, and (C₁-C₆)alkyl, which is optionally substituted with 1 to 3 substituents independently selected from the group consisting of Y₁, Y₂ and Y₃, where t is 0, 1, 2, or 3, wherein when t is 2 or 3, the CR³R⁴ units may be the same or different;
R² is selected from the group consisting of hydrogen, halogen, and (C₁-C₆)alkyl optionally substituted with 1-3 substituents independently selected from the group consisting of Y₁, Y₂ and Y₃;
R³ and R⁴ are independently selected from the group consisting of H, F, and (C₁-C₆)alkyl, or wherein R³ and R⁴, are selected together to form a carbocycle, or two R³ groups on adjacent carbon atoms are selected together to form a carbocycle;
wherein each Y₁, Y₂, and Y₃ is independently selected and is
(i) selected from the group consisting of H, halogen, cyano, nitro, tetrazolyl, guanidino, amidino, azido, —C(O)Z₁, methylguanidino, —CF₃, —CF₂CF₃, —CH(CF₃)₂, —C(OH)(CF₃)₂, —OCF₃, —OCF₂H, —OCF₂CF₃, —OC(O)NH₂, —OC(O)NHZ₁, —OC(O)NZ₁Z₂, —NHC(O)Z₁, —NHC(O)NH₂, —NHC(O)NZ₁, —NHC(O)NZ₁Z₂, —C(O)OH, —C(O)OZ₁, —C(O)NH₂, —C(O)NHZ₁, —C(O)NZ₁Z₂, —P(O)₃H₂, —P(O)₃(Z₁)₂, —S(O)₃H, —S(O)ₘZ₁, —Z₁, —OZ₁, —OH, —NH₂, —NHZ₁, —NZ₁Z₂, —C(═NH)NH₂, —C(═NOH)NH₂, —N-morpholino, (C₂-C₆)alkenyl, (C₂-C₆)alkynyl, (C₁-C₆)haloalkyl, (C₂-C₆)haloalkenyl, (C₂-C₆)haloalkynyl, (C₁-C₆)haloalkoxy, —(CZ₃Z₄)ᵣNH₂, —(CZ₃Z₄)ᵣNHZ₁, —(CZ₃Z₄)ᵣNZ₁Z₂, and —S(O)ₘ(CF₂)_{q}CF₃, wherein m is 0, 1 or 2, q is an integer from 0 to 5, r is an integer from 1 to 4, Z₁ and Z₂ are independently selected from the group consisting of alkyl of 1 to 12 carbon atoms, cycloalklyl of 3 to 8 carbon atoms, aryl of 6 to 14 carbon atoms, heterocycle of 5 to 14 ring atoms, aralkyl of 7 to 15 carbon atoms, and heteroaralkyl of 5 to 14 ring atoms, and Z₃ and Z₄ are independently selected from the group consisting of hydrogen, alkyl of 1 to 12 carbon atoms, aryl of 6 to 14 carbon atoms, heteroaryl of 5 to 14 ring atoms, aralkyl of 7 to 15 carbon atoms, and heteroaralkyl of 5 to 14 ring atoms;
(ii) Y₁ and Y₂ are selected together to be —O[C(Z₃)(Z₄)]ᵣO— or —O[C(Z₃)(Z₄)]ᵣ₊₁—; or
(iii) when any two of Y₁, Y₂, or Y₃ are attached to the same or adjacent atoms, are selected together to form a carbocycle or heterocycle;
and wherein any of the above-mentioned substituents comprising a CH₃ (methyl), CH₂ (methylene), or CH (methine) group which is not attached to a halogen, SO or SO₂ group or to a N, O or S atom optionally bears on said group a substituent selected from hydroxy, halogen, (C₁-C₄)alkyl, (C₁-C₄)alkoxy and —N[(C₁-C₄)alkyl][(C₁-C₄)alkyl];
or a pharmaceutically acceptable prodrug, pharmaceutically active metabolite, pharmaceutically acceptable solvate or pharmaceutically acceptable salt thereof.

2. A compound, pharmaceutically acceptable prodrug, pharmaceutically active metabolite, pharmaceutically acceptable solvate or pharmaceutically acceptable salt of claim 1, wherein R¹ is a 5- or 6-membered aryl or heteroaryl group, optionally substituted with 1-3 substituents independently selected from the group consisting of Y₁, Y₂ and Y₃.

3. A compound, pharmaceutically acceptable prodrug, pharmaceutically active metabolite, pharmaceutically acceptable solvate or pharmaceutically acceptable salt of claim 1, having the structure of Formula (II): 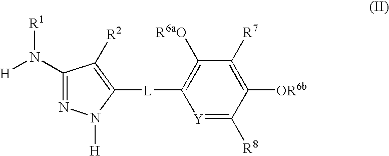
wherein Y is CR⁵ or N;
R^{6a} and R^{6b} are selected from the group consisting of H, —C(O)R⁹, —C(O)OR¹⁰, —C(O)NR⁹R¹⁰ and a moiety selected from the group consisting of —(CR³R⁴)ᵤ-aryl, —(CR³R⁴)ᵤ-heterocycle, —(CR³R⁴)ᵤ—(C₃-C₆)cycloalkyl, (C₂-C₆)alkenyl, and (C₁-C₆)alkyl, optionally substituted with 1 to 3 substituents independently selected from the group consisting of Y₁, Y₂ and Y₃; where u is 0, 1, 2, or 3, wherein when u is 2 or 3, the CR³R⁴ units may be the same or different;
each of R⁵, R⁷, and R⁸ is independently selected from the group consisting of H, halogen, methyl, ethyl, —CN, —CF₃, and —C(O)CH₃;
each of R⁹ and R¹⁰ is independently selected from the group consisting of —(CR³R⁴)ᵤ-aryl, —(CR³R⁴)ᵤ-heterocycle, —(CR³R⁴)ᵤ—(C₃-C₆)cycloalkyl, (C₂-C₆)alkenyl, and (C₁-C₆)alkyl, optionally substituted with 1 to 3 substituents independently selected from the group consisting of Y₁, Y₂ and Y₃; where u is 0, 1, 2, or 3, wherein when u is 2 or 3, the CR³R⁴ units may be the same or different.

4. The compound, pharmaceutically acceptable prodrug, pharmaceutically active metabolite, pharmaceutically acceptable solvate or pharmaceutically acceptable salt of claim 2, wherein L is selected from the group consisting of

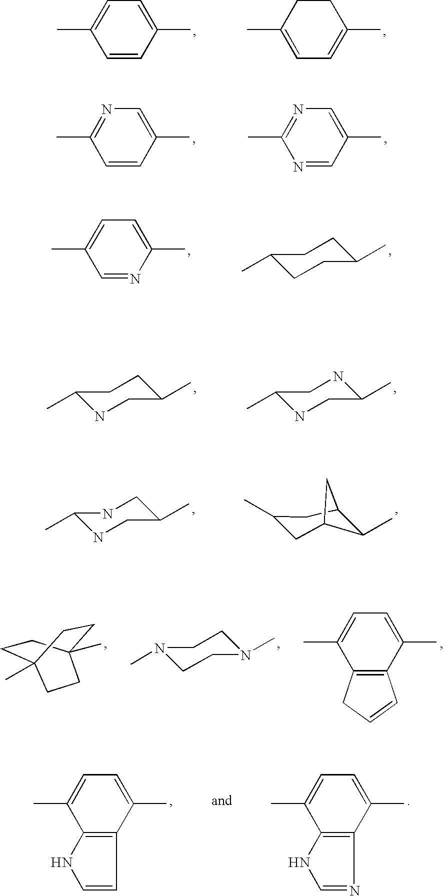

5. A compound, pharmaceutically acceptable prodrug, pharmaceutically active metabolite, pharmaceutically acceptable solvate or pharmaceutically acceptable salt of claim 1, wherein L and Ar are each an independently selected optionally substituted phenyl or pyridyl group.

6. A compound, pharmaceutically acceptable prodrug, pharmaceutically active metabolite, pharmaceutically acceptable solvate or pharmaceutically acceptable salt of claim 5, wherein Ar^{x} is

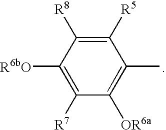

7. A compound, pharmaceutically acceptable prodrug, pharmaceutically active metabolite, pharmaceutically acceptable solvate or pharmaceutically acceptable salt of claim 6, having the structure:

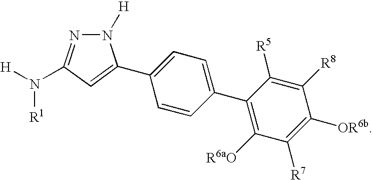

8. A compound, pharmaceutically acceptable prodrug, pharmaceutically active metabolite, pharmaceutically acceptable solvate or pharmaceutically acceptable salt of claim 7, where R¹ has the structure: 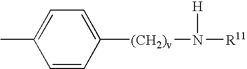
wherein v is 0, 1, or 2; and wherein R¹¹ is (C₁-C₆)alkyl.

9. A compound according to claim 1 selected from the group consisting of:
3-{[3-(2′,4′-Dihydroxy-1,1′-biphenyl-4-yl)-1H-pyrazol-5-yl]amino}benzonitrile;
4-{[3-(2′,4′-Dihydroxy-1,1′-biphenyl-4-yl)-1H-pyrazol-5-yl]amino}benzonitrile;
4′-[5-(3-Cyclopropylaminomethyl-phenylamino)-2H-pyrazol-3-yl]-biphenyl-2,4-diol acetate;
4′-[5-(4-Cyclopropylaminomethyl-phenylamino)-2H-pyrazol-3-yl]-biphenyl-2,4-diol acetate;
4′-[5-(4-isoPropylaminomethyl-phenylamino)-2H-pyrazol-3-yl]-biphenyl-2,4-diol acetate;
4′-[5-(4-N-Cyclopropylaminomethyl-phenylamino)-2H-pyrazol-3-yl]-biphenyl-5-methyl-2,4-diol;
4′-[5-(4-N-Cyclopropylaminomethyl-phenylamino)-2H-pyrazol-3-yl]-biphenyl-6-methyl-2,4-diol;
4′-[5-(4-Cyclopropylaminomethyl-phenylamino)-2H-pyrazol-3-yl]-biphenyl-6-chloro-2,4-diol acetate;
4′-[5-({4-[(cyclopropylamino)methyl]phenyl}amino)-1H-pyrazol-3-yl]-6-fluoro-1,1′-biphenyl-2,4-diol;
4′-[5-(4-Cyclopropylmethylaminomethyl-phenylamino)-2H-pyrazol-3-yl]-biphenyl-2,4-diol acetate;
N-[3-(2′,4′-dihydoxy-1,1′-biphenyl-4-yl)-1H-pyrazol-5-yl]pyrimidin-2-amine;
4′-[5-(6-Hydroxymethyl-pyridin-3-ylamino)-2H-pyrazol-3-yl]-biphenyl-2,4-diol;
4′-[5-(2-Hydroxymethyl-pyridin-4-ylamino)-2H-pyrazol-3-yl]-biphenyl-2,4-diol acetate;
4′-[5-({6-[(cyclopentylamino)methyl]pyridin-3-yl}amino)-1H-pyrazol-3-yl]-1,1′-biphenyl-2,4-diol;
4′-[5-({6-[(dimethylamino)methyl]pyridin-3-yl}amino)-1H-pyrazol-3-yl]-1,1′-biphenyl-2,4-diol;
5-[5-(2′,4′-Dihydroxy-biphenyl-4-yl)-2H-Pyrazol-3-ylamino]-pyridine-2-carbothioic acid methylamide;
N-[5-(2′,4′-Dihydroxy-1,1′-biphenyl-4-yl)-1H-pyrazol-3-yl]pyridin-2-amine;
N-[5-(2′,4′-Dihydroxy-1,1′-biphenyl-4-yl)-1H-pyrazol-3-yl]pyridin-3-amine;
4′-[5-(pyridin-4-ylamino)-1H-pyrazol-3-yl]-1,1′-biphenyl-2,4-diol;
4′-[5-(1,3-thiazol-5-ylamino)-1H-pyrazol-3-yl]-1,1′-biphenyl-2,4-diol;
4′-(3-anilino-1H-pyrazol-5-yl)-1′-biphenyl-2,4-diol;
4′-{5-[(6-{[(cyclopropylmethyl)amino]methyl}pyridin-3-yl)amino]-1H-pyrazol-3-yl}-1,1′-biphenyl-2,4-diol;
4′-[5-({6-[(cyclopropylamino)methyl]pyridin-3-yl}amino)-1H-pyrazol-3-yl]-1,1′-biphenyl-2,4-diol;
4′-[5-({6-[(isopropylamino)methyl]pyridin-3-yl}amino)-1H-pyrazol-3-yl]-1,1′-biphenyl-2,4-diol; and
-[5-({6-[(ethylamino)methyl]pyridin-3-yl}amino)-1H-pyrazol-3-yl]-1,1′-biphenyl-2,4-diol;
or a pharmaceutically acceptable prodrug, pharmaceutically active metabolite, pharmaceutically acceptable solvate or pharmaceutically acceptable salt thereof.

10. A compound according to claim 1 selected from the group consisting of: 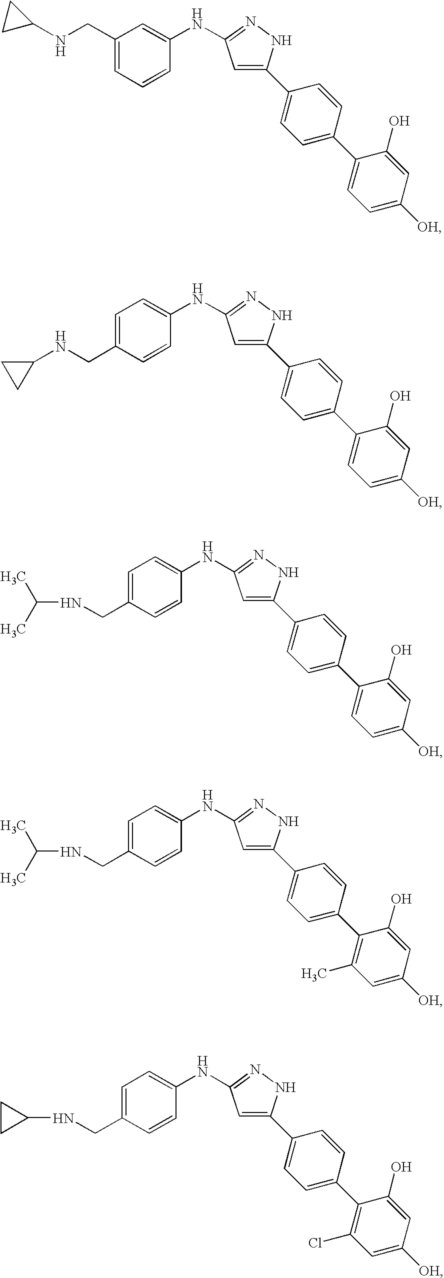 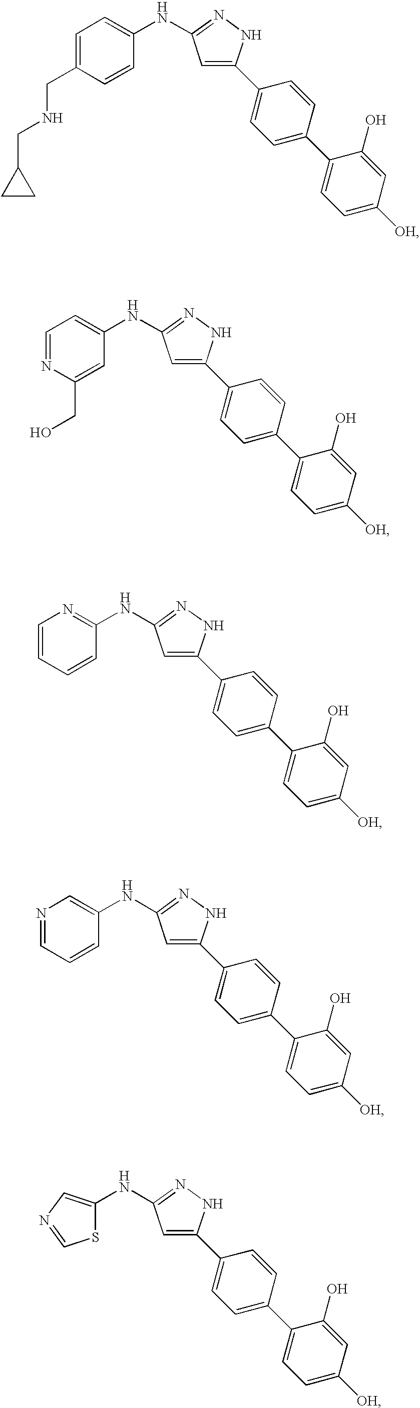 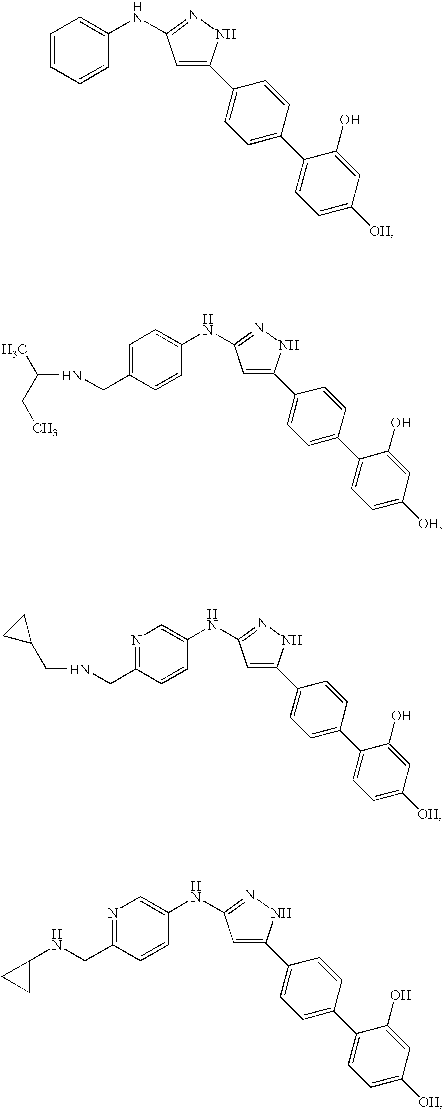 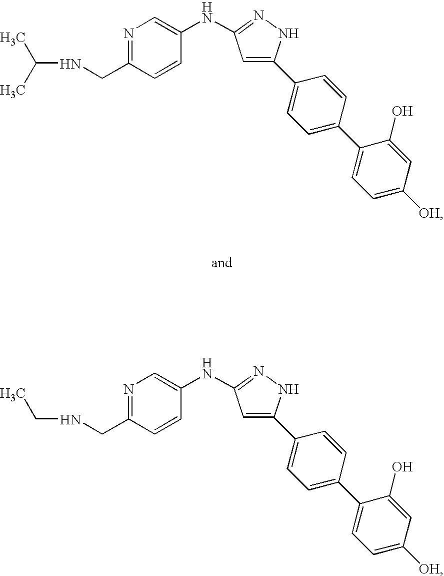
or a pharmaceutically acceptable prodrug, pharmaceutically active metabolite, pharmaceutically acceptable solvate or pharmaceutically acceptable salt thereof.

11. A method of modulating the activity of a protein kinase receptor, comprising contacting the kinase receptor with an effective amount of a compound, pharmaceutically acceptable prodrug, pharmaceutically active metabolite, pharmaceutically acceptable solvate or pharmaceutically acceptable salt as defined in claim 1.

12. The method of claim 11 wherein the protein kinase is CHK1.

13. A pharmaceutical composition for the treatment of a hyperproliferative disorder in a mammal comprising an enhancing effective amount of a compound, prodrug, metabolite, salt or solvate of claim 1 and a pharmaceutically acceptable carrier.

14. The pharmaceutical composition of claim 13, wherein said hyperproliferative disorder is cancer.

15. The pharmaceutical composition of claim 14, wherein said cancer is brain, lung, kidney, renal, ovarian, ophthalmic, squamous cell, bladder, gastric, pancreatic, breast, head, neck, oesophageal, gynecological, prostate, colorectal or thyroid cancer.

16. The pharmaceutical composition of claim 13, wherein said hyperproliferative disorder is noncancerous.

17. The pharmaceutical composition of claim 16, wherein said hyperproliferative disorder is a benign hyperplasia of the skin or prostate.

18. A pharmaceutical composition for the treatment of a hyperproliferative disorder in a mammal comprising an enhancing effective amount of a compound, prodrug, metabolite, salt or solvate of claim 1 in combination with an anti-neoplastic agent.

19. The pharmaceutical composition of claim 18 wherein the anti-neoplastic agent is capable of damaging DNA in a malignant cell.

20. The pharmaceutical composition of claim 18 wherein the anti-neoplastic agent is selected from the group consisting of mitotic inhibitors, alkylating agents, anti-metabolites, intercalating antibiotics, enzymes, topoisomerase inhibitors, biological response modifiers, anti-hormones, and anti-androgens, and a pharmaceutically acceptable carrier.

21. A method of treating a hyperproliferative disorder in a mammal comprising administering to said mammal an enhancing effective amount of a compound, prodrug, metabolite, salt or solvate of claim 1.

22. The method of claim 21 wherein said hyperproliferative disorder is cancer.

23. The method of claim 22 wherein said cancer is brain, lung, ophthalmic, squamous cell, renal, kidney, ovarian, bladder, gastric, pancreatic, breast, head, neck, oesophageal, prostate, colorectal, gynecological or thyroid cancer.

24. The method of claim 21 wherein said hyperproliferative disorder is noncancerous.

25. The method of claim 24 wherein said hyperproliferative disorder is a benign hyperplasia of the skin or prostate.

26. A method for the treatment of a hyperproliferative disorder in a mammal comprising administering to said mammal an enhancing effective amount of a compound, prodrug, metabolite, salt or solvate of claim 1 in combination with an anti-neoplastic agent.

27. The method of claim 26 wherein the anti-neoplastic agent is capable of damaging DNA in a malignant cell.

28. The method of claim 26 wherein the anti-neoplastic agent is selected from the group consisting of mitotic inhibitors, alkylating agents, anti-metabolites, intercalating antibiotics, growth factor inhibitors, cell cycle inhibitors, enzymes, topoisomerase inhibitors, biological response modifiers, anti-hormones, and anti-androgens.

29. A method of treating a mammalian disease condition mediated by protein kinase activity, comprising administering to a mammal in need thereof a therapeutically effective amount of a compound, pharmaceutically acceptable prodrug, pharmaceutically active metabolite, pharmaceutically acceptable solvate, or pharmaceutically acceptable salt as defined in claim 1.

30. The method of claim 29, wherein the mammalian disease condition is associated with tumor growth, cell proliferation, or angiogenesis.

31. A compound according to claim 1, wherein Ar is

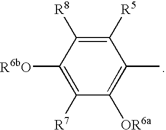

32. A compound according to claim 31 wherein R₁ is phenyl or pyridyl.

33. A compound according to claim 32 wherein R₁ is mono-substituted with Y₁.

34. A compound according to claim 33 wherein R₇ and R₈ are hydrogen.

35. A compound according to claim 34 wherein Y₁ is —(CZ₃Z₄)ᵣNHZ₁ or —NHZ₁.

36. A compound according to claim 35 wherein R₅ is hydrogen or halogen.

37. A compound according to claim 36 wherein R^{6a} and R^{6b} are hydrogen.

38. A compound according to claim 37 wherein L is 1,4-phenylene.

39. A compound according to claim 34 wherein L is 1,4-phenylene.

40. A compound according to claim 1 wherein L is 1,4-phenylene.

41. A compound according to claim 40 wherein Ar is

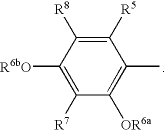

42. A compound according to claim 41 wherein R¹ is phenyl or pyridyl.

43. A compound according to claim 42 wherein R¹ is unsubstituted or mono-substituted with Y₁.

44. A compound according to claim 43 wherein R⁷ is 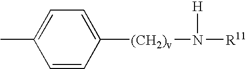
wherein v is 0, 1or 2 and R¹¹ is (C₆-C₆) alkyl.

45. A compound according to claim 1 having the structure:

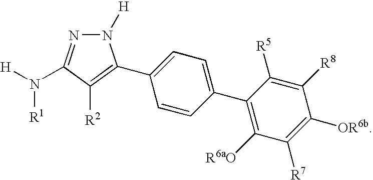

46. A compound according to claim 1 having the structure: 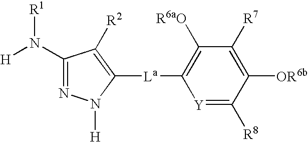
wherein L is Lₐ which is a rigid linking group that orients the aminopyrazole moiety linearly or near-linearly with a resorcinol or resorcinol-like moiety.

47. A compound according to claim 46 wherein R^{6a} and R^{6b} are selected from the group consisting of H, —C(O)R⁹, —C(O)OR¹⁰, 13 C(O)NR⁹R¹⁰ and a moiety selected from the group consisting of (C₃-C₆)cycloalkyl, —(CH₂)ᵤphenyl, —(CH₂)ᵤheterocycle and (C₁-C₄)alkyl which is optionally substituted with 1 to 3 substituents independently selected from the group consisting of Y₁, Y₂ and Y₃, where R⁹ and R¹⁰ are optionally substituted from the group consisting of (C₃-C₆)cycloalkyl, —(CH₂)ᵤphenyl and (C₁-C₆)alkyl which are optionally substituted with 1 to 3 substituents independently selected from the group consisting of Y₁, Y₂ and Y₃; and each of R⁵, R⁷ and R⁸ are independently hydrogen or halogen.
